(19) United States
(12) Patent Application Publication
(10) Pub. No.: US 2011/0124840 A1
(43) Pub. Date: May 26, 2011

(54) Synthesis of Homopolymers and Block Copolymers

(76) Inventors: Kurt Breitenkamp, Tampa, FL (US); Kevin N. Sill, Tampa, FL (US)

(21) Appl. No.: 11/884,219

(22) PCT Filed: Feb. 8, 2006
(86) PCT No.: PCT/US2006/004160

§ 371 Date: Mar. 31, 2008

Related U.S. Application Data
(60) Provisional application No. 60/652,251, filed on Feb. 11, 2005.

(51) Int. Cl. C07K 14/00

(52) U.S. Cl. 530/324

(57) ABSTRACT

The present invention relates to the field of polymer chemistry and more particularly to homopolymers and block copolymers and methods of preparing the same.

### CROSS-REFERENCE TO RELATED APPLICATIONS

This application claims priority to U.S. provisional application Ser. No. 60/652,251, filed Feb. 11, 2005, the entirety of which is hereby incorporated herein by reference.

### FIELD OF THE INVENTION

The present invention relates to the field of polymer chemistry and more particularly to homopolymers and block copolymers, uses thereof, and intermediates thereto.

### BACKGROUND OF THE INVENTION

Homopolymers and block copolymers having a poly(amino acid) portion are of great synthetic interest. The poly(amino acid) portion of such polymers is typically prepared by the ring-opening polymerization of an amino acid-N-carboxy-anhydride (NCA). However, methods for preparing the poly(amino acid) block that employ free amines as initiators of the NCA polymerization afford homopolymers or block copolymers with a wide range of polydispersity indices (PDIs) that tend to be quite high. For example, Schlaad reported PDI values of 1.12-1.60 by initiating polymerization with amino-terminated polystyrene. Schlaad (2003 Eur. Chem. J.) also reports a PDI of 7.0 for crude PEG-b-poly(L-benzyl glutamate) copolymers and a PDI of 1.4 after fractionation. Chen (Biomaterials, 2004) reported a PDI of 1.5 for poly(ε-caprolactone) (PCL)-b-poly(ethylene glycol) (PEG)-b-poly (γ-benzyl-L-glutamate)(PBLG). Rodriguez-Hernández reported the synthesis of pH-responsive poly(L-glutamic acid)-b-poly(L-lysine) copolymers synthesized using a small molecule amine initiator (J. Am. Chem Soc., 2005). In this case, polymers initiated using hexylamine possessed PDI values of ˜1.4. It is believed that these high PDIs are due to the highly reactive nature of the NCAs.

To date, there have been only a few reported synthetic methods to prepare homopolymers or block copolymers that contain a poly(amino acid) portion with a narrower distribution of molecular weights. These include amine-initiated NCA polymerization utilizing high vacuum techniques developed by Hadjichristidis (Biomacromolecules, 2004), and the nickel-catalyzed coordination-insertion polymerization of NCAs developed by Darning at the University of California-Santa Barbara (see U.S. Pat. No. 6,686,446). Poly(amino acids) synthesized using high vacuum techniques are synthetically challenging to prepare, employ handmade reaction vessels, and require long time periods for reagent purification and complete polymerization to be achieved. Due to these factors, only a few grams of poly(amino acid) can be prepared in a single polymerization reaction. In addition, since homopolymers or block copolymers that comprise a poly(amino acid) portion are typically designed for biological applications, the use of organometallic initiators and catalysts is undesirable.

Another method for the controlled polymerization of an NCA, initiated amine salt, was first reported by Schlaad and coworkers (Chem. Comm., 2003, 2944-2945). It is believed that, during the reaction, the chain end exists primarily in its unreactive salt form as a dormant species and that the unreactive amine salt is in equilibrium with the reactive amine. The free amine is capable of ring opening the NCA, which adds one repeat unit to the polymer chain. This cycle repeats until all of the monomer is consumed and the final poly(amino acid) is formed. This reported method has limitations in that only a single poly(amino acid) block is incorporated. In addition, this reported method only described the use of a polystyrene macroinitator. In another publication by Schlaad and coworkers (Eur. Phys. J., 2003, 10, 17-23), the author indicates that use of a PEG macroiniator results in diverse and unpredictable PDIs. The author further indicates that even “the coupling of preformed polymer segments like that of a haloacylated poly(ethylene oxide) with poly(L-aspartic acid) . . . yields block copolymers that are chemically disperse and are often contaminated with homopolymers.”

Whereas each of the methods described above relates to initiating the ring opening polymerization (ROP) of NCAs using a synthetic polymer having a terminal amine group, Aliferis and coworkers reported the ROP of NCAs using the primary amines n-hexylamine and 1,6-diaminohexane. See Aliferis, et al., “Living Polypeptides”, Biomacromolecules, 2004, 5, 1653-1656. However, the method described by Aliferis involved highly stringent vacuum techniques in order to control the amine-initiated polymerization of NCAs.

Accordingly, there remains a need for a facile method for preparing homopolymers or block copolymers comprising a poly(amino acid) portion wherein the method is well controlled and one or more poly(amino acid) blocks are incorporated.

### DETAILED DESCRIPTION OF CERTAIN EMBODIMENTS OF THE INVENTION

### 1. General Description

The present invention provides methods for the synthesis of homopolymers or block copolymers containing a non-polymeric core portion and one or more poly(amino acid) blocks. The poly(amino acid) portions of these homopolymers or block copolymers are prepared by controlled ring-opening polymerization of cyclic monomers such as N-carboxy anhydrides (NCAs), lactams, and cyclic imides, wherein the polymerization is initiated by a non-polymeric amine salt. Such amine salt initiators may be prepared by protonation of small molecule amines. Without wishing to be bound by any particular theory, it is believed that the amine salt reduces or eliminates many side reactions that are commonly observed with traditional polymerization of these reactive monomers. This leads to homopolymers or block copolymers with narrow distributions of block lengths and molecular weights. It has been surprisingly found that the sequential addition of monomers provides multi-block copolymers having desirable low polydispersity.

The sequential addition of cyclic monomers to a “living” polymer chain end (i.e. a terminal amine salt) affords multi-block copolymers having a variety of poly(amino acid) block types. Accordingly, one aspect of the present invention provides a method for preparing a multi-block copolymer comprising a non-polymeric core portion and one or more different poly(amino acid) blocks, wherein said method comprises the step of sequentially polymerizing one or more different cyclic amino acid monomers onto a non-polymeric amine salt wherein said polymerization is initiated by said amine salt.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “sequential polymerization”, and variations thereof, refers to the method wherein, after a first monomer (e.g. NCA, lactam, or imide) is incorporated into the polymer, thus forming an amino acid “block”, a second monomer (e.g. NCA, lactam, or imide) is added to the reaction to form a second amino acid block, which process may be continued in a similar fashion to introduce additional amino acid blocks into the resulting multi-block copolymers. In certain embodiments, the term “block”, as used herein, includes those formed from a random mixture of two amino acids. For example, such “blocks” may comprise a mixture of two or more hydrophobic or two or more hydrophilic monomers.

As used herein, the term “non-polymeric core portion”, and variations thereof, refers to the non-polymeric amine salt that initiates polymerization of the first monomer and thus becomes incorporated into the product obtained therefrom. For example, if butylamine hydrochloride is used as the non-polymeric amine salt for initiating polymerization of the first monomer, it will be appreciated that the butyl moiety will become the non-polymeric core resulting from that polymerization.

As used herein, the term “homopolymer” refers to a polymer comprising a single poly(amino acid) portion. The term “block copolymer” refers to a polymer comprising at least two poly(amino acid) portions. The term “multi-block copolymer” refers to a polymer comprising two or more differing poly(amino acid) portions. These are also referred to as diblock copolymers (e.g., having two differing poly(amino acid) portions), triblock copolymers (e.g., having three differing poly(amino acid) portions), etc. Such block copolymers and copolymers include those having the format X-W-X, X-W-X′, W-X-X′, W-X-X′-X″, X′-X-W-X-X′, X′-X-W-X″-X′″, or W-X-X′-X, wherein W is the non-polymeric core portion and X, X′, X″ and X′″ are differing poly(amino acid) portions. In certain aspects, the non-polymeric core portion is used as the center block, which allows the growth of multiple blocks symmetrically from center, examples of which have the format X-W-X and X′-X-W-X-X′.

As used herein, the term “synthetic polymer” refers to a polymer that is not a poly(amino acid). Such synthetic polymers are well known in the art and include polystyrene, polyalkylene oxides, such as poly(ethylene oxide) (also referred to as polyethylene glycol or PEG), polyesters (polycaprolactone, polylactic acid, etc.), polyphosphazenes, poly(2-hydroxylethyl acrylate), poly(2-hydroxyethyl methacrylate), poly(ethyleneimine), poly(N-isopropyl acrylamide), Duncan's Polymers, and derivatives thereof.

As used herein, the term “poly(amino acid)” refers to a covalently linked amino acid chain wherein each monomer is an amino acid unit. Such amino acid units include natural and unnatural amino acids. In certain embodiments, each amino acid unit is in the L-configuration. Such poly(amino acids) include those having suitable protecting groups. For example, amino acid monomers may have hydroxyl or amino moieties, which are optionally protected by a suitable hydroxyl protecting group or a suitable amine protecting group, as appropriate. Such suitable hydroxyl protecting groups and suitable amine protecting groups are described in more detail herein, infra. As used herein, an amino acid block comprises one or more monomers or a set of two or more monomers. In certain embodiments, an amino acid block comprises one or more monomers such that the overall block is hydrophilic. In other embodiments, an amino acid block comprises one or more monomers such that the overall block is hydrophobic. In still other embodiments, amino acid blocks of the present invention include random amino acid blocks, i.e., blocks comprising a mixture of amino acid residues.

As used herein, the phrase “natural amino acid side-chain group” refers to the side-chain group of any of the 20 amino acids naturally occurring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyroine nonpolar and hydrophobic by virtue of protecting the hydroxyl group.

As used herein, the phrase “unnatural amino acid side-chain group” refers to amino acids not included in the list of 20 amino acids naturally occurring in proteins, as described above. Such amino acids include the D-isomer of any of the 20 naturally occurring amino acids. Unnatural amino acids also include homoserine, ornithine, and thyroxine. Other unnatural amino acids side-chains are well know to one of ordinary skill in the art and include unnatural aliphatic side chains. Other unnatural amino acids include modified amino acids, including those that are N-alkylated, cyclized, phosphorylated, acetylated, amidated, labelled, and the like.

As used herein, the phrase “living polymer chain-end” refers to the terminus resulting from a polymerization reaction which maintains the ability to react further with additional monomer or with a polymerization terminator.

As used herein, the term “termination” refers to attaching a terminal group to a polymer chain-end by the reaction of a living polymer with an appropriate compound. Alternatively, the term “termination” may refer to attaching a terminal group to an amine or hydroxyl end, or derivatives thereof, of the polymer chain.

As used herein, the term “polymerization terminator” is used interchangeably with the term “polymerization terminating agent” and refers to a compound that reacts with a living polymer chain-end to afford a polymer with a terminal group. Alternatively, the term “polymerization terminator” may refer to a compound that reacts with an amine or hydroxyl end, or derivative thereof, of the polymer chain, to afford a polymer with a terminal group.

As used herein, the term “polymerization initiator” refers to a compound, or amine and/or amine salt thereof, which reacts with the desired monomer in a manner which results in polymerization of that monomer. In certain embodiments, the polymerization initiator is the amine salt described herein.

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of =saturation, but which is not aromatic. Unless otherwise specified, aliphatic groups contain 1-20 carbon atoms. In some embodiments, aliphatic groups contain 1-10 carbon atoms. In other embodiments, aliphatic groups contain 1-8 carbon atoms. In still other embodiments, aliphatic groups contain 1-6 carbon atoms, and in yet other embodiments aliphatic groups contain 1-4 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon. This includes any oxidized form of nitrogen, sulfur, phosphorus, or silicon; the quaternized form of any basic nitrogen, or; a substitutable nitrogen of a heterocyclic ring including ═N— as in 3,4-dihydro-2H-pyrrolyl, —NH— as in pyrrolidinyl, or ═N(R^{†})— as in N-substituted pyrrolidinyl.

The term “unsaturated”, as used herein, means that a moiety has one or more units of unsaturation.

The term “aryl” used alone or as part of a larger moiety as in “aralkyl”, “aralkoxy”, or “aryloxyalkyl”, refers to monocyclic, bicyclic, and tricyclic ring systems having a total of five to fourteen ring members, wherein at least one ring in the system is aromatic and wherein each ring in the system contains three to seven ring members. The term “aryl” may be used interchangeably with the term “aryl ring”.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O—(CH₂)₀₋₄C(O)OR^{o}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘}C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; (CH₂)₀₋₄(O)₂OR^{∘}; —(CH₂)₀₋₄OS(O)₂R^{∘}; —S(O)₂NR^{∘}₂; —(CH₂)₀₋₄(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched)alkylene) O—N(R^{∘})₂; or —(C₁₋₄ straight or branched)alkylene) C(O)O—N(R^{∘})₂, wherein each R^{∘}may be substituted as defined below and is independently hydrogen, C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R⁵⁰⁰ , -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, SiR^{•}₃, —OSiR^{•}₃, —C(O)SR^{•}, —(C₁₋₄ straight or branched alkylene) C(O)OR^{•}, or —SSR^{•}wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur. Suitable divalent substituents on a saturated carbon atom of R^{∘}include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from, nitrogen, oxygen, or sulfur. A suitable tetravalent substituent that is bound to vicinal substitutable methylene carbons of an “optionally substituted” group is the dicobalt hexacarbonyl cluster represented by

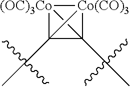

when depicted with the methylenes which bear it.

Suitable substituents on the aliphatic group of R* include halogen, —R^{•}, -(halonR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{\}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl)acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

A “crown ether moiety” is the radical of a crown ether. A crown ether is a monocyclic polyether comprised of repeating units of —CH₂CH₂O—. Examples of crown ethers include 12-crown-4,15-crown-5, and 18-crown-6.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays and in neutron scattering experiments.

As used herein, the term “detectable moiety” is used interchangeably with the term “label” and relates to any moiety capable of being detected (e.g., primary labels and secondary labels). A “detectable moiety” or “label” is the radical of a detectable compound.

“Primary” labels include radioisotope-containing moieties (e.g., moieties that contain ³²P, ³³P, ³⁵S, or ¹⁴C), mass-tags, and fluorescent labels, and are signal-generating reporter groups which can be detected without further modifications.

Other primary labels include those useful for positron emission tomography including molecules containing radioisotopes (e.g. ¹⁸F) or ligands with bound radioactive metals (e.g. ⁶²Cu). In other embodiments, primary labels are contrast agents for magnetic resonance imaging such as gadolinium, gadolinium chelates, or iron oxide (e.g Fe₃O₄ and Fe₂O₃) particles. Similarly, semiconducting nanoparticles (e.g. cadmium selenide, cadmium sulfide, cadmium telluride) and core-shell semiconducting nanoparticles (e.g. cadmium selenide (core)/zinc sulfide (shell), cadmium selenide (core)/zinc selenide (shell)) are useful as fluorescent labels. Other metal nanoparticles (e.g colloidal gold) also serve as primary labels.

“Secondary” labels include moieties such as biotin, or protein antigens, that require the presence of a second compound to produce a detectable signal. For example, in the case of a biotin label, the second compound may include streptavidin-enzyme conjugates. In the case of an antigen label, the second compound may include an antibody-enzyme conjugate. Additionally, certain fluorescent groups can act as secondary labels by transferring energy to another compound or group in a process of nonradiative fluorescent resonance energy transfer (FRET), causing the second compound or group to then generate the signal that is detected.

Unless otherwise indicated, radioisotope-containing moieties are optionally substituted hydrocarbon groups that contain at least one radioisotope. Unless otherwise indicated, radioisotope-containing moieties contain from 1-40 carbon atoms and one radioisotope. In certain embodiments, radioisotope-containing moieties contain from 1-20 carbon atoms and one radioisotope.

The terms “fluorescent label”, “fluorescent group”, “fluorescent compound”, “fluorescent dye”, and “fluorophore”, as used herein, refer to compounds or moieties that absorb light energy at a defined excitation wavelength and emit light energy at a different wavelength. Examples of fluorescent compounds include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “substrate”, as used herein refers to any material or macromolecular complex to which a functionalized end-group of a homopolymer or block copolymer can be attached. Examples of commonly used substrates include, but are not limited to, glass surfaces, silica surfaces, plastic surfaces, metal surfaces, surfaces containing a metallic or chemical coating, membranes (eg., nylon, polysulfone, silica), micro-beads (eg., latex, polystyrene, or other polymer), porous polymer matrices (eg., polyacrylamide gel, polysaccharide, polymethacrylate), macromolecular complexes (eg., protein, polysaccharide).

### 3. Description of Exemplary Embodiments

As described generally above, one aspect of the present invention provides a method for preparing a homopolymer or block copolymer comprising a non-polymeric core and one or more different poly(amino acid) blocks, wherein said method comprises the step of sequentially polymerizing one or more different cyclic amino acid monomers onto a non-polymeric amine salt wherein said polymerization is initiated by said amine salt. In certain embodiments, said polymerization occurs by ring-opening polymerization of the cyclic amino acid monomers. In other embodiments, the cyclic amino acid monomer is an amino acid NCA, lactam, or cyclic imide.

As described generally above, the non-polymeric core used in the methods of the present invention has an amine salt for initiating the polymerization of a cyclic amino acid monomer. Such salts include the acid addition salts of an amino group formed with an inorganic acid such as hydrochloric acid, hydrobromic acid, phosphoric acid, sulfuric acid or perchloric acid. It is also contemplated that such amine salts include the acid addition salts of an amino group formed with an organic acid such as acetic acid, oxalic acid, maleic acid, tartaric acid, citric acid, succinic acid, malonic acid, methanesulfonic acid, phenylsulfonic acid, optionally substituted phenylsulfonic acids, sulfinic acid, phenylsulfinic acid, optionally substituted phenylsulfinic acid, trifluoroacetic acid, triflic acid, benzoic acid, optionally substituted benzoic acids, and the like, or by using other methods used in the art such as ion exchange. Further amine salts include, when appropriate, ammonium, and amine cations formed using counterions such as halide, hydroxide, carboxylate, sulfate, phosphate, nitrate, lower alkyl sulfonate and aryl sulfonate.

Another aspect of the present invention provides a method of preparing a homopolymer or block copolymer comprising one or more different poly(amino acid) blocks and a non-polymeric core moiety R¹, wherein said method comprises the steps of:
(a) providing a compound of formula I:

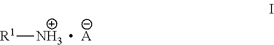

wherein:
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and

A is a suitable acid anion,
(b) polymerizing a first cyclic amino acid monomer onto the amine salt terminal end of formula I; and
(c) optionally polymerizing additional cyclic amino acid monomers onto the living polymer end.

In other embodiments, the present invention provides a method for preparing a block copolymer comprising two or more different poly(amino acid) blocks and a non-polymeric core moiety R¹, wherein said method comprises the steps of:
(a) providing a compound of formula I:

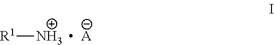

wherein:
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and

A is a suitable acid anion,
(b) polymerizing a first cyclic amino acid monomer onto the amine salt terminal end of formula I;
(c) polymerizing a second cyclic amino acid monomer onto the living polymer end, wherein said second cyclic amino acid monomer is different from said first cyclic amino acid monomer; and
(d) optionally polymerizing additional cyclic amino acid monomers onto the living polymer end.

In certain embodiments, the cyclic amino acid monomers include N-carboxy anhydrides (NCAs), lactams, and cyclic imides. According to one embodiment, the cyclic amino acid monomer is an NCA. NCAs are well known in the art and are typically prepared by the carbonylation of amino acids by a modification of the Fuchs-Farthing method (Kricheldorf, α-Aminoacid-N-Carboxy-Anhydrides and Related Heterocycles: Syntheses, Properties, Peptide Synthesis, Polymerization, 1987). Although reaction conditions vary among different amino acids, most, if not all, natural and unnatural, 2-substituted amino acids can be converted to N-carboxy anhydrides using phosgene gas or triphosgene (for ease of handling). It will be appreciated that, although α-amino acids are described below, one of ordinary skill in the art would recognize that NCAs may be prepared from β- and γ-amino acids as well. In addition, NCAs can be prepared from dimers or trimers of amino acids. Using an amino acid having an R^{x} side-chain, as defined herein, as an example, Scheme 1 below depicts the typical formation of an NCA using phosgene.

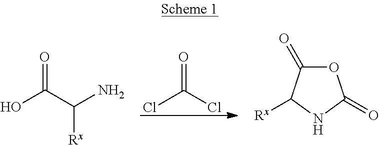

NCAs exhibit reactivity that is well-suited for ring-opening polymerization (ROP). Primary, secondary, and tertiary amines as well as alcohols, water, and acid are known to initiate the ring opening of the NCA.

Because a wide variety of functionalities can initiate the polymerizations of NCAs, amino acids containing alcohol, amine, and carboxylic acid functionality are typically protected before polymerization. Such protected hydroxyl groups, protected amine groups, and protected carboxylic acids are well known in the art and include those described above and in Greene (1999).

Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl.

Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like. In certain embodiments, the amino protecting group is phthalimido. In other embodiments, the amino protecting group is mono- or di-benzyl or mono- or di-allyl. In still other embodiments, the amino protecting group is a tert-butyloxycarbonyl (BOC) group.

Suitable carboxylate protecting groups include, but are not limited to, substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, tert-butyl, benzyl, and phenyl wherein each group is optionally substituted.

Both D and L NCA enantiomers can be synthesized and any combination of the two stereoisomers can undergo ring-opening polymerization. Advanced Chemtech (http://www.advancedchemtech.com) and Bachem (www.bachem.com) are commercial and widely-referenced sources for both protected and unprotected amino acids. It will be appreciated that amino acid dimers and trimers can form cyclic anhydrides and are capable of ROP in accordance with the present invention.

In certain embodiments, the cyclic amino acid monomer is a carboxylate-protected aspartic acid NCA, a hydroxyl-protected tyrosine NCA, or an amino-protected lysine NCA. In other embodiments, the cyclic amino acid monomer is a t-butyl protected aspartic acid NCA, a benzyl-protected tyrosine NCA, or a BOC-protected lysine NCA. In yet other embodiments, a mixture of cyclic amino acid monomers, such as a hydroxyl-protected tyrosine NCA and phenylalanine NCA, are polymerized simultaneously to form polymer blocks comprising two different amino acids.

According to another embodiment, the cyclic amino acid monomer is a lactam. Lactams are another class of monomers that can be polymerized by cationic ROP. (Odian, Principles of Polymerization, Ch. 7) Such lactams suitable for the present invention include the four, five (pyrrolidone), six (piperidone) and seven (caprolactam) member rings depicted below:

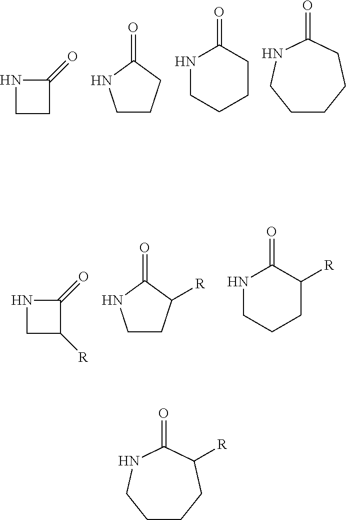

wherein each R is independently halogen; N₃, CN, R^{∘}; OR^{∘}; SR^{∘}; phenyl (Ph) optionally substituted with R^{∘}; —O(Ph) optionally substituted with R^{∘}; (CH₂)₁₋₂(Ph), optionally substituted with R^{∘}; CH═CH(Ph), optionally substituted with R^{∘}; NO₂; CN; N(R^{∘})₂; NR^{∘}C(O)R^{∘}; NR^{∘}C(O)N(R^{∘})₂; NR^{∘}CO₂R^{∘}; NR^{∘}NR^{∘}C(O)R^{∘}; NR^{∘}NR^{∘}C(O)N(R^{∘})₂; NR^{∘}NR^{∘}CO₂R^{∘}; C(O)C(O)R^{∘}; C(O)CH₂C(O)R^{∘}; CO₂R^{∘}; C(O)R^{∘}; C(O)N(R^{∘})₂; OC(O)N(R^{∘})₂; S(O)₂R^{∘}; SO₂N(R^{∘})₂; S(O)R^{∘}; NR^{∘}SO₂N(R^{∘})₂; NR^{∘}SO₂R^{∘}; C(═S)N(R^{∘})₂; C(═NH)—N(R^{∘})₂; or (CH₂)₀₋₂NHC(O)R^{∘} wherein each R^{∘} may be substituted as defined below and is independently hydrogen, C₁₋₆ aliphatic, —(CH₂)₀₋₁Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{∘}, on the same substituent or different substituents, taken together with the atom(s) to which each R^{∘} group is bound, form a 3-8-membered cycloalkyl, heterocyclyl, aryl, or heteroaryl ring having 0-4 heteroatoms independently selected from nitrogen, oxygen, or sulfur. Suitable monovalent substituents on R^{∘}, are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —C(O)SiR^{•}₃, —(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Substitution α to the amide carbonyl allows for incorporation of almost unlimited types of chemical functionality into the polymer backbone. As is the case with NCA polymerization, potential nucleophiles incorporated into the monomer are protected prior to prevent any undesired branching reactions.

As described generally above, the R¹ group of formula I includes an optionally substituted C₁₋₁₀ aliphatic group. Such aliphatic groups, as defined herein, include straight, branched, saturated, and unsaturated groups. In certain embodiments, the R¹ group of formula I is an optionally substituted straight chain aliphatic group. Exemplary substituents on the R¹ group of formula I include —N₃, —CN, an amino group or salt or protected form thereof, a protected aldehyde group, a protected hydroxyl group, a protected carboxylic acid group, a protected thiol group, an optionally substituted aliphatic group, or a detectable moiety.

Exemplary compounds of formula I wherein R¹ is an optionally substituted straight chain aliphatic group include:

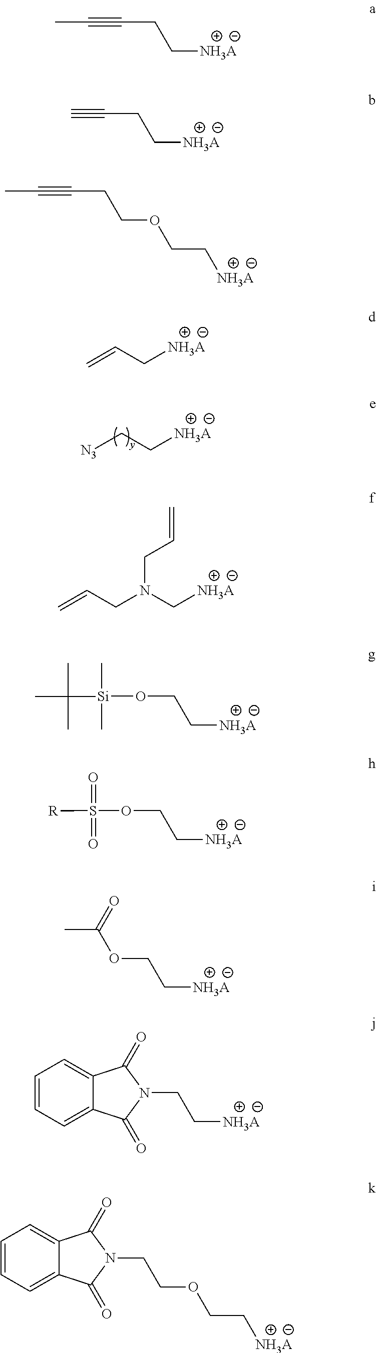

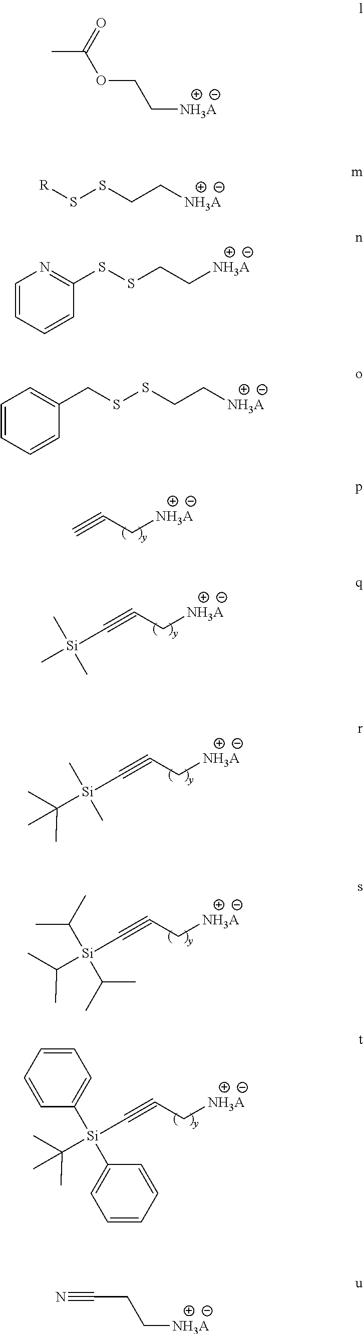

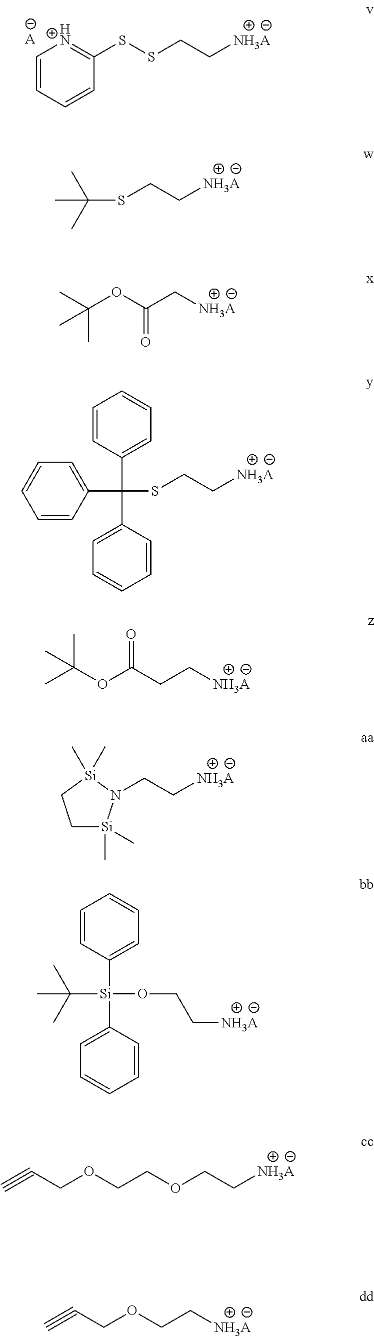

wherein each y is independently 1-6.

As described generally above, the R¹ group of formula I includes an optionally substituted group selected from 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, or a 12-14 membered saturated, partially unsaturated, or aryl tricyclic ring having 0-5 heteroatoms independently selected from oxygen, nitrogen, or sulfur. In certain embodiments, R¹ is an optionally substituted group selected from 5-7 membered saturated, partially unsaturated, or aryl monocyclic ring having 0-2 heteroatoms independently selected from oxygen, nitrogen, or sulfur. In other embodiments, R¹ is a 9-10 membered saturated, partially unsaturated, or aryl bicyclic ring having 0-3 heteroatoms independently selected from oxygen, nitrogen, or sulfur. In still other embodiments, R¹ is a 13-14 membered saturated, partially unsaturated, or aryl tricyclic ring having 0-3 heteroatoms independently selected from oxygen, nitrogen, or sulfur. Such cyclic R¹ groups, as defined herein, include optionally substituted phenyl, pyridyl, naphthyl, quinolinyl, isoquinolinyl, quinazolinyl, anthracenyl, and the like.

In certain embodiments, the R¹ group of formula I is an optionally substituted 5-6 membered saturated, partially unsaturated, or aryl monocyclic ring having 0-3 heteroatoms independently selected from oxygen, nitrogen, or sulfur. In other embodiments, the R¹ group of formula I is an optionally substituted phenyl group. Exemplary substituents on the R¹ groups of formula I include —N₃, —CN, an amino group, a mono-protected amino group, a di-protected amino group, a protected aldehyde group, a protected hydroxyl group, a protected carboxylic acid group, a protected thiol group, an optionally substituted aliphatic group, or a detectable moiety.

Exemplary compounds of formula I wherein R¹ is an optionally substituted cyclic group include:

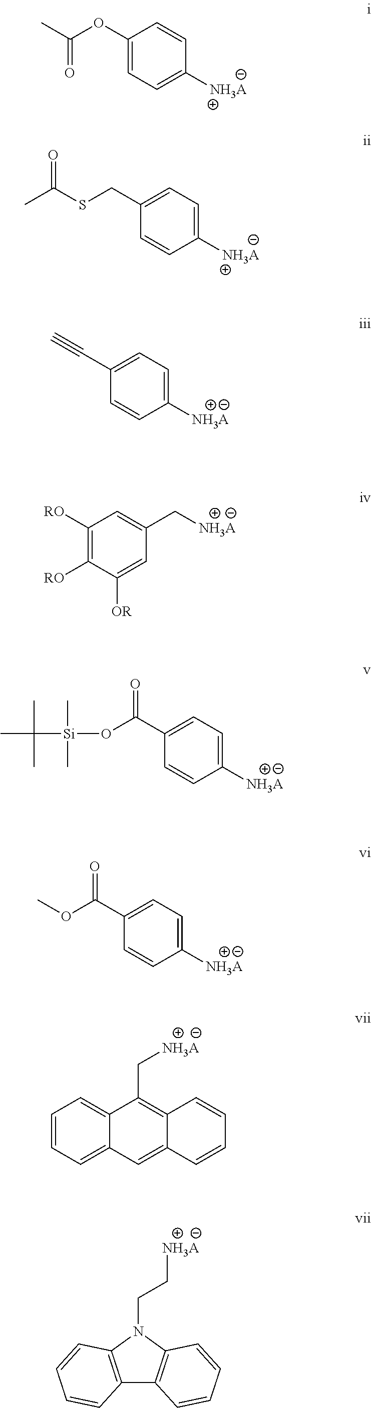

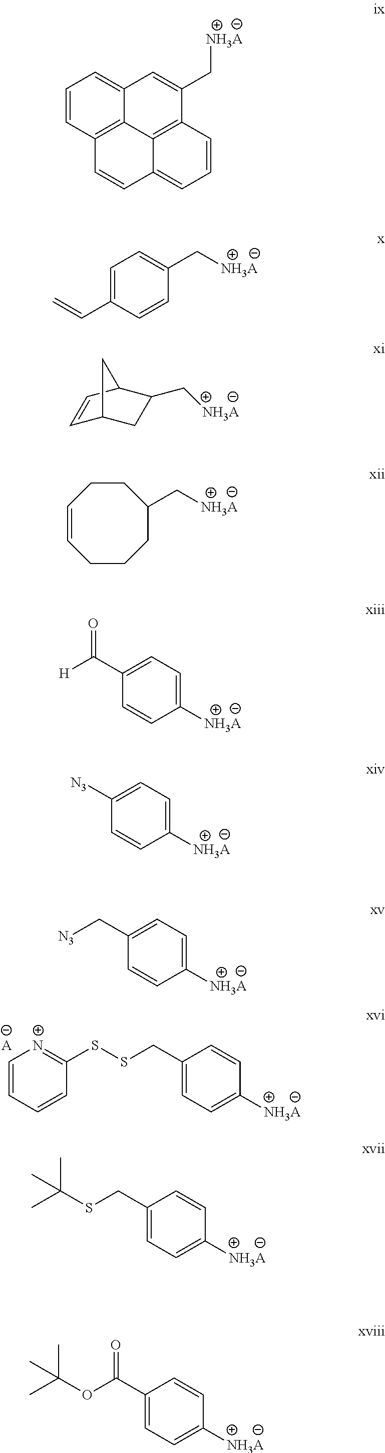

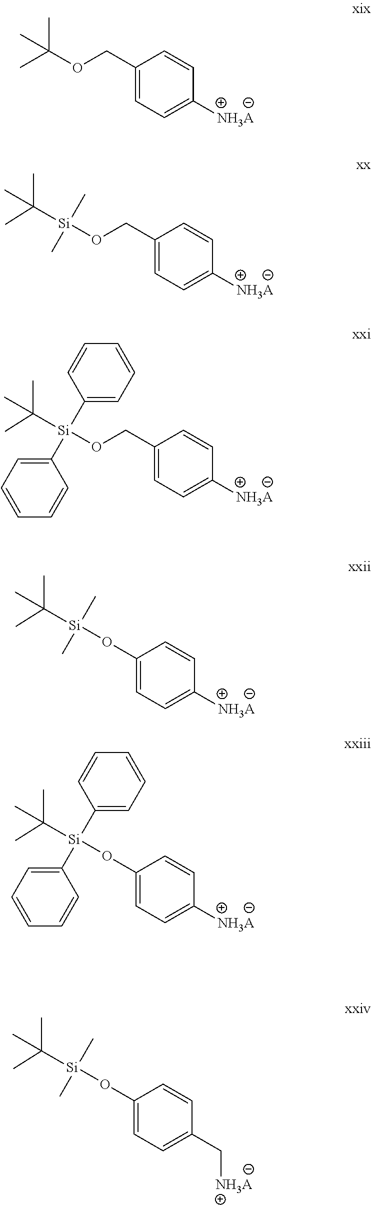

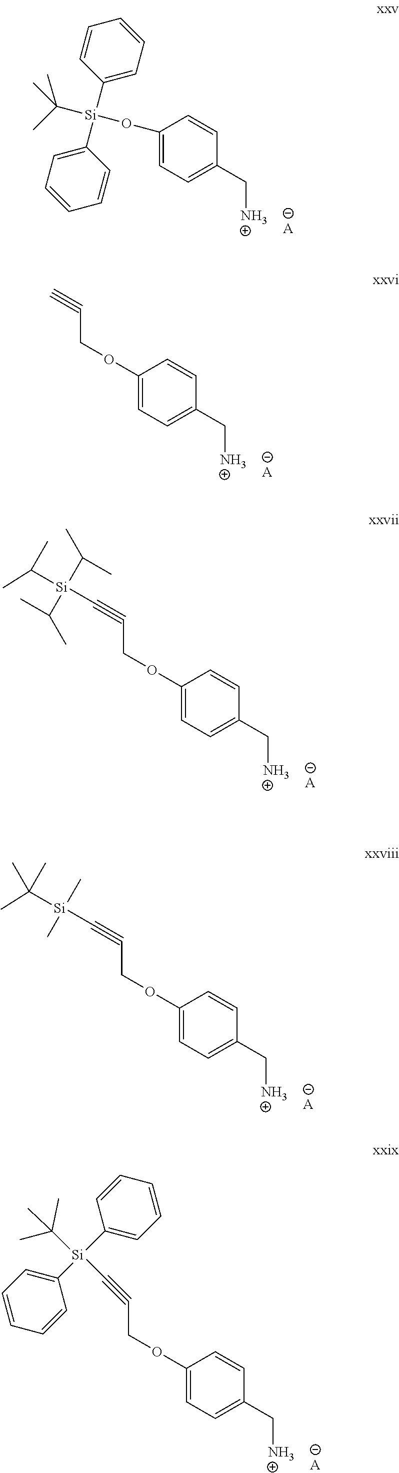

wherein each R is as defined generally above and in classes and subclasses described above and herein.

In certain embodiments, the R¹ group of formula I comprises a fluorescent moiety.

In certain embodiments, the R¹ aliphatic group of formula I comprises a group suitable for Click chemistry. Click reactions tend to involve high-energy (“spring-loaded”) reagents with well-defined reaction coordinates, giving rise to selective bond-forming events of wide scope. Examples include the nucleophilic trapping of strained-ring electrophiles (epoxide, aziridines, aziridinium ions, episulfonium ions), certain forms of carbonyl reactivity (aldehydes and hydrazines or hydroxylamines, for example), and several types of cycloaddition reactions. The azide-alkyne 1,3-dipolar cycloaddition is one such reaction. Click chemistry is known in the art and one of ordinary skill in the art would recognize that certain R¹ moieties of the present invention are suitable for Click chemistry.

Compounds of formula I having R¹ moieties, suitable for Click chemistry are useful for conjugating said compounds to biological systems or macromolecules such as proteins, viruses, and cells, to name but a few. The Click reaction is known to proceed quickly and selectively under physiological conditions. In contrast, most conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Thus, another embodiment of the present invention provides a method of conjugating the R¹ group of a compound of formula I to a macromolecule via Click chemistry. Yet another embodiment of the present invention provides a macromolecule conjugated to a compound of formula I via the R¹ group.

Multi-block copolymers of the present invention may be of the form X-W-X′, W-X-X′, W-X-X′-X″, X′-X-W-X-X′, X′-X-W-X″-X′″, or W-X-X′-X. For example, when W is a non-polymeric core having two terminal amine salts, a first cyclic amino acid monomer X may be polymerized onto the amine salt terminal ends of W. A second cyclic amino acid monomer X′ may then be polymerized onto the resulting amine salts thus forming a multi-block copolymer of the form X′-X-W-X-X′, wherein W is a non-polymeric core and X and X′ are differing poly(amino acid) chains. In an alternate example, when W is a non-polymeric core having one terminal amine salt and one protected-amine terminus, a first cyclic amino acid monomer X may be polymerized onto the amine salt terminal end of W, following which, the protected amine, at the other terminus, may be deprotected and the corresponding amine salt formed. A second cyclic amino acid monomer X′ may then be polymerized onto the resulting amine salt thus forming a multi-block copolymer of the form X-W-X′, wherein W is a non-polymeric core and X and X′ are differing poly(amino acid) chains.

Before or after incorporating the poly (amino acid) block portions into the multi-block coploymer of the present invention resulting in a multi-block copolymer of the form W-X-X′, the other end-group functionality, corresponding to a substituent on the R¹ moiety of formula I, can be used to attach targeting groups for cell specific delivery including, but not limited to, detectable moieties, such as fluorescent dyes, covalent attachment to surfaces, and incorporation into hydrogels. Alternatively, such a substituent on the R¹ moiety of formula I can be bonded to a biomolecule, drug, cell, or other suitable substrate.

Another aspect of the present invention provides a method for preparing a multi-block copolymer of formula II:

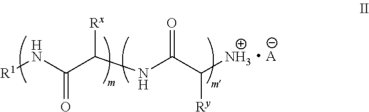

wherein:
m is 1-1000;
m′ is 0-1000;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
A is a suitable acid anion; and
R^{x} and R^{y} are each independently a natural or unnatural amino acid side-chain group, wherein R^{x} and R^{y} are different from each other,
   wherein said method comprises the steps of:
(a) providing a compound of formula I:

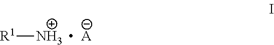

wherein:
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and
A is a suitable acid anion,
(b) polymerizing a first cyclic amino acid monomer onto the amine salt terminal end of formula I, wherein said first cyclic amino acid monomer comprises R^{x}; and
(c) optionally polymerizing a second cyclic amino acid monomer, comprising R^{y}, onto the living polymer end, wherein said second cyclic amino acid monomer is different from said first cyclic amino acid monomer.

Another aspect of the present invention provides a compound of formula II:

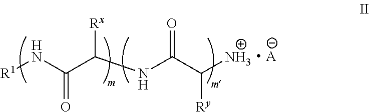

wherein:
m is 1-1000;
m′ is 0-1000;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
A is a suitable acid anion; and
R^{x} and R^{y} are each independently a natural or unnatural amino acid side-chain group, wherein R^{x} and R^{y} are different from each other,
   wherein said compound is prepared by a method comprising the steps of
(a) providing a compound of formula I:

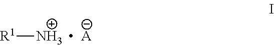

wherein:
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and
A is a suitable acid anion,
(b) polymerizing a first cyclic amino acid monomer onto the amine salt terminal end of formula I, wherein said first cyclic amino acid monomer comprises R^{x}; and
(c) optionally polymerizing a second cyclic amino acid monomer, comprising R^{y}, onto the living polymer end, wherein said second cyclic amino acid monomer is different from said first cyclic amino acid monomer.

According to another embodiment, the present invention provides a compound of formula II:

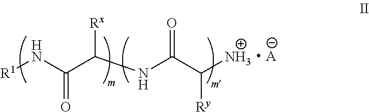

wherein:
m is 1-1000;
m′ is 0-1000;
R^{x} and R^{y} are each independently a natural or unnatural amino acid side-chain group, wherein R^{x} and R^{y} are different from each other;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and
A is a suitable acid anion.

In certain embodiments, the R¹ group of formula II is substituted with —N₃.

In certain embodiments, the R¹ group of formula II is an optionally substituted aliphatic group. In some embodiments, said R¹ moiety is an optionally substituted alkyl group. In other embodiments, said R¹ moiety is an optionally substituted alkynyl or alkenyl group. When said R¹ moiety is a substituted aliphatic group, suitable substituents on R¹ include CN, a mono-protected amino group, a di-protected amino group, a protected aldehyde group, a protected hydroxyl group, a protected carboxylic acid group, a protected thiol group, or a detectable moiety.

In certain embodiments, R¹ is an optionally substituted group selected from 5-7 membered saturated, partially unsaturated, or aryl monocyclic ring having 0-2 heteroatoms independently selected from oxygen, nitrogen, or sulfur. In other embodiments, R¹ is a 9-10 membered saturated, partially unsaturated, or aryl bicyclic ring having 0-3 heteroatoms independently selected from oxygen, nitrogen, or sulfur. In still other embodiments, R¹ is a 13-14 membered saturated, partially unsaturated, or aryl tricyclic ring having 0-3 heteroatoms independently selected from oxygen, nitrogen, or sulfur. Such cyclic R¹ groups, as defined herein, include optionally substituted phenyl, naphthyl, and anthracenyl groups.

In certain embodiments, the R¹ group of formula II is an optionally substituted 5-6 membered saturated, partially unsaturated, or aryl monocyclic ring having 0-3 heteroatoms independently selected from oxygen, nitrogen, or sulfur. In other embodiments, the R¹ group of formula II is an optionally substituted phenyl group. Exemplary substituents on the R¹ groups of formula II include —N₃, —CN, an amino group, a mono-protected amino group, a di-protected amino group, a protected aldehyde group, a protected hydroxyl group, a protected carboxylic acid group, a protected thiol group, an optionally substituted aliphatic group, or a detectable moiety.

In certain embodiments, the R¹ group of formula II comprises a fluorescent moiety.

In other embodiments, the R¹ group of formula II is substituted with a protected hydroxyl group. In certain embodiments the protected hydroxyl of the R¹ moiety is an ester, carbonate, sulfonate, allyl ether, ether, silyl ether, alkyl ether, arylalkyl ether, or alkoxyalkyl ether. In certain embodiments, the ester is a formate, acetate, proprionate, pentanoate, crotonate, or benzoate. Exemplary esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Exemplary carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Exemplary alkyl ethers include methyl, benzyl, p-methoxybenzyl, 3,4-dimethoxybenzyl, trityl, t-butyl, and allyl ether, or derivatives thereof. Exemplary alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Exemplary arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

In certain embodiments, the R¹ group of formula II is substituted with a mono-protected or di-protected amino group. In certain embodiments R¹ is a mono-protected amine. In certain embodiments R¹ is a mono-protected amine selected from aralkylamines, carbamates, allyl amines, or amides. Examplary mono-protected amino moieties include t-butyloxycarbonylamino, ethyloxycarbonylamino, methyloxycarbonylamino, trichloroethyloxy-carbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino. In other embodiments R¹ is a di-protected amine. Exemplary di-protected amines include di-benzylamine, di-allylamine, phthalimide, maleimide, succinimide, pyrrole, 2,2,5,5-tetramethyl-[1,2,5]azadisilolidine, and azide. In certain embodiments, the R¹ moiety is phthalimido. In other embodiments, the R¹ moiety is mono- or di-benzylamino or mono- or di-allylamino. In certain embodiments, the R¹ group is 2-dibenzylaminoethoxy.

In other embodiments, the R¹ group of formula II is substituted with a protected aldehyde group. In certain embodiments the protected aldehydro moiety of R¹ is an acyclic acetal, a cyclic acetal, a hydrazone, or an imine. Exemplary R¹ groups include dimethyl acetal, diethyl acetal, diisopropyl acetal, dibenzyl acetal, bis(2-nitrobenzyl)acetal, 1,3-dioxane, 1,3-dioxolane, and semicarbazone. In certain embodiments, R¹ is an acyclic acetal or a cyclic acetal. In other embodiments, R¹ is a dibenzyl acetal.

In yet other embodiments, the R¹ group of formula II is substituted with a protected carboxylic acid group. In certain embodiments, the protected carboxylic acid moiety of R¹ is an optionally substituted ester selected from C₁₋₆ aliphatic or aryl, or a silyl ester, an activated ester, an amide, or a hydrazide. Examples of such ester groups include methyl, ethyl, propyl, isopropyl, butyl, isobutyl, benzyl, and phenyl ester. In other embodiments, the protected carboxylic acid moiety of R¹ is an oxazoline or an ortho ester. Examples of such protected carboxylic acid moieties include oxazolin-2-yl and 2-methoxy-[1,3]dioxin-2-yl. In certain embodiments, the R¹ group is oxazolin-2-ylmethoxy or 2-oxazolin-2-yl-1-propoxy.

According to another embodiments, the R¹ group of formula II is substituted with a protected thiol group. In certain embodiments, the protected thiol of R¹ is a disulfide, thioether, silyl thioether, thioester, thiocarbonate, or a thiocarbamate. Examples of such protected thiols include triisopropylsilyl thioether, t-butyldimethylsilyl thioether, t-butyl thioether, benzyl thioether, p-methylbenzyl thioether, triphenylmethyl thioether, and p-methoxyphenyldiphenylmethyl thioether. In other embodiments, R¹ is an optionally substituted thioether selected from alkyl, benzyl, or triphenylmethyl, or trichloroethoxycarbonyl thioester. In certain embodiments, R¹ is —S—S-pyridin-2-yl, —S—SBn, —S—SCH₃, or —S—S(p-ethynylbenzyl). In other embodiments, R¹ is —S—S-pyridin-2-yl. In still other embodiments, the R¹ group is 2-triphenylmethylsulfanyl-ethoxy.

In other embodiments, the R¹ group of formula II is substituted with a crown ether moiety. Examplary crown ether moieties include radicals of 12-crown-4,15-crown-5, and 18-crown-6.

In still other embodiments, the R¹ group of formula II is substituted with a detectable moiety. According to one aspect of the invention, the R¹ group of formula II is substituted with a fluorescent moiety. Such fluorescent moieties are well known in the art and include coumarins, quinolones, benzoisoquinolones, hostasol, and Rhodamine dyes, to name but a few. Exemplary fluorescent moieties of the R¹ group include anthracen-9-yl, pyren-4-yl, 9-H-carbazol-9-yl, the carboxylate of rhodamine B, and the carboxylate of coumarin 343. In other embodiments, the R¹ group of formula II is a fluorescent moiety.

In certain embodiments, the R¹ group of formula II is substituted with a group suitable for Click chemistry. Click reactions tend to involve high-energy (“spring-loaded”) reagents with well-defined reaction coordinates, that give rise to selective bond-forming events of wide scope. Examples include nucleophilic trapping of strained-ring electrophiles (epoxide, aziridines, aziridinium ions, episulfonium ions), certain carbonyl reactivity (e.g., the reaction between aldehydes and hydrazines or hydroxylamines), and several cycloaddition reactions. The azide-alkyne 1,3-dipolar cycloaddition is one such reaction. Click chemistry is known in the art and one of ordinary skill in the art would recognize that certain R¹ substituents of the present invention are suitable for Click chemistry.

Compounds of formula II having R¹ substituents suitable for Click chemistry are useful for conjugating said compounds to biological systems or macromolecules such as proteins, viruses, and cells, to name but a few. The Click reaction is known to proceed quickly and selectively under physiological conditions. In contrast, most conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Thus, another embodiment of the present invention provides a method of conjugating the R¹ group of a compound of formula II to a macromolecule via Click chemistry. Yet another embodiment of the present invention provides a macromolecule conjugated to a compound of formula II via the R¹ group.

Before or after conjugation of compounds of formula II to a biomolecule, drug, cell, substrate, or the like via R¹, the other end-group functionality, corresponding to free amine or salt thereof, group of formula II, can be used to attach targeting groups for cell-specific delivery including, but not limited to, detectable moieties, such as fluorescent dyes, covalent attachment to surfaces, and incorporation into hydrogels.

According to one embodiment, the R¹ group of formula II is substituted with an azide-containing group. According to another embodiment, the R¹ group of formula II is an alkyne-containing group. In certain embodiments, the R¹ group of formula II has a terminal alkyne moiety. In other embodiments, the R¹ group of formula II is an alkyne moiety having an electron withdrawing group. Accordingly, in such embodiments, the R¹ group of formula II is

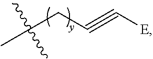

wherein E is an electron withdrawing group and y is 0-6. Such electron withdrawing groups are known to one of ordinary skill in the art. In certain embodiments, E is an ester. In other embodiments, the R¹ group of formula II is

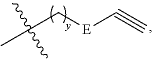

wherein E is an electron withdrawing group, such as a —C(O)O— group and y is 0-6.

In certain embodiments, m′ is 0. In other embodiments, m and m′ are each independently 1-1000. According to other embodiments, m and m′ are each independently 10 to 100. In still other embodiments, m is 1-20, and m′ is 10-50.

In certain embodiments, one of R^{x} and R^{y} is a hydrophilic, or crosslinkable, amino acid side-chain group, or suitably protected form thereof, and the other of R^{x} and R^{y} is a hydrophobic, or ionic amino acid side-chain group, or suitably protected form thereof. In other embodiments, R^{x} is a hydrophilic or crosslinkable amino acid side-chain group and R^{y} is a hydrophobic, or ionic amino acid side-chain group. Such hydrophilic, or crosslinkable, amino acid side-chain groups include tyrosine, serine, cysteine, threonine, aspartic acid (also known as aspartate, when charged), glutamic acid (also known as glutamate, when charged), asparagine, and glutamine. Such hydrophobic amino acid side-chain groups include a suitably protected tyrosine side-chain, a suitably protected serine side-chain, a suitably protected threonine side-chain, phenylalanine, alanine, valine, leucine, tryptophan, proline, benzyl and alkyl glutamates, or benzyl and alkyl aspartates or mixtures thereof. Such ionic amino acid side chain groups includes a lysine side-chain, arginine side-chain, or a suitably protected lysine or arginine side-chain, an aspartic acid side chain, glutamic acid side-chain, or a suitably protected aspartic acid or glutamic acid side-chain. One of ordinary skill in the art would recognize that protection of a polar or hydrophilic amino acid side-chain can render that amino acid nonpolar. For example, a suitably protected tyrosine hydroxyl group can render that tyrosine nonpolar and hydrophobic by virtue of protecting the hydroxyl group. Suitable protecting groups for the hydroxyl, amino, and thiol, and carboylate functional groups of R^{x} and R^{y} are as described herein.

In other embodiments, R^{y} comprises a mixture of hydrophobic and hydrophilic amino acid side-chain groups such that the overall poly(amino acid) block comprising R^{y} is hydrophobic. Such mixtures of amino acid side-chain groups include phenylalanine/tyrosine, phenalanine/serine, leucine/tyrosine, and the like. According to another embodiment, R^{y} is a hydrophobic amino acid side-chain group selected from phenylalanine, alanine, or leucine, and one or more of tyrosine, serine, or threonine.

In other embodiments, one or both of R^{x} and R^{y} comprise functional groups capable of forming cross-links. According to another embodiment, R^{x} comprises a functional group capable of forming cross-links. It will be appreciated that a variety of functional groups are capable of such cross-linking, including, but not limited to, carboxylate, hydroxyl, thiol, and amino groups. Examples of NCA's having functional groups capable of forming cross-links, or protected forms thereof, include protected forms of glutamic and aspartic acid, such as:

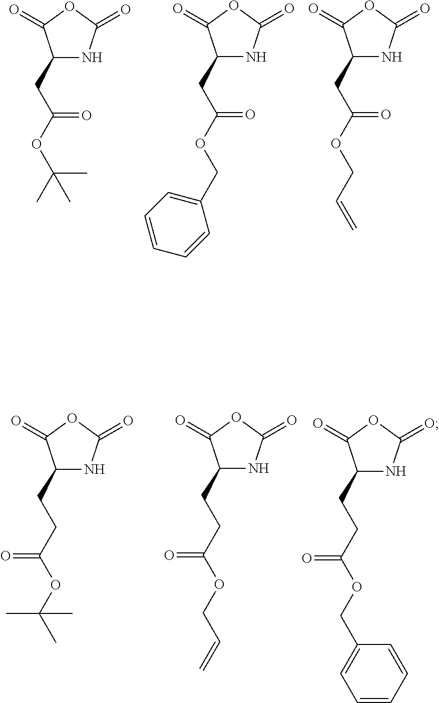

protected forms of cysteine capable of forming disulfide crosslinking via the corresponding thiol, such as:

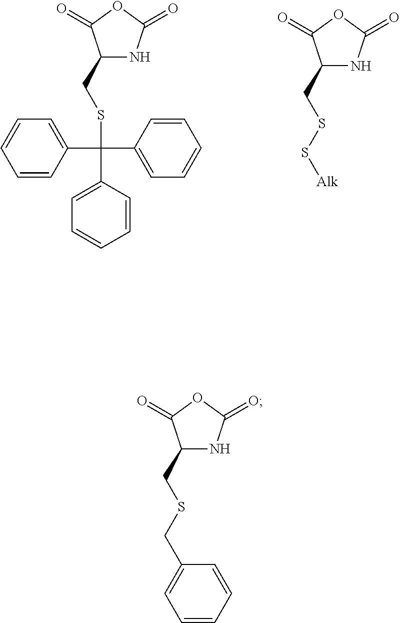

protected forms of serine capable of glutaraldehyde crosslinking via the corresponding hydroxyl, such as:

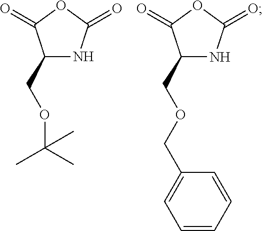

and
NCAs that contain aldehyde moieties capable of glutaraldehyde crosslinking, or protected forms thereof, such as:

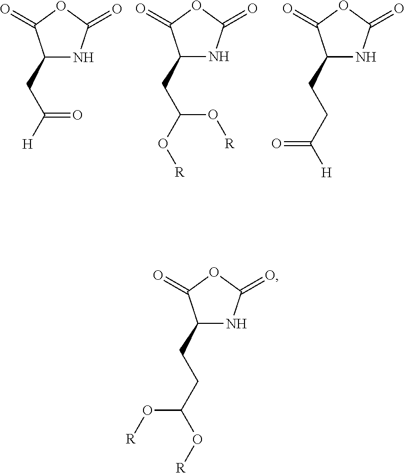

wherein R is R^{†} as defined herein, supra.

Other nonlimiting examples of amino acid monomers suitable for the methods of the present invention include protected forms of aspartic and glutamic acid, such as:

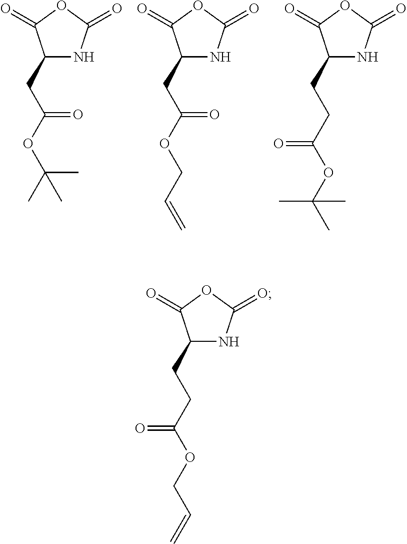

protected forms of lysine, such as:

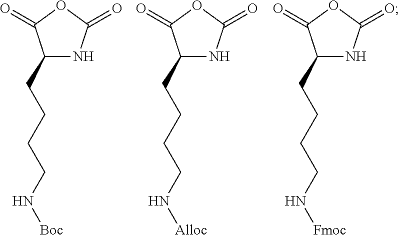

protected forms of arginine, such as:

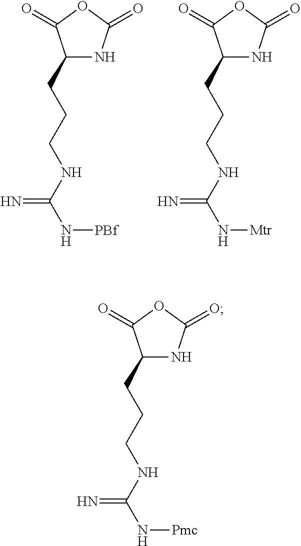

and
protected forms of histidine, such as:

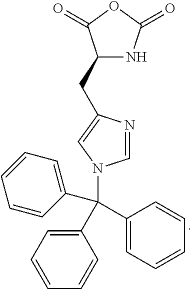

It will be appreciated that in addition to each of the protecting groups depicted above, a variety of carboxyl and amino protecting groups, as described in more detail herein, are suitable.

One skilled in the art will recognize that the amine salts of formula II can be treated with a suitable base to provide the corresponding free amine. Accordingly, another embodiment of the present invention provides a compound of formula II′:

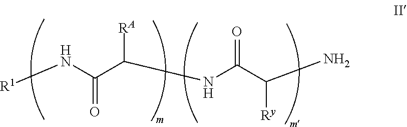

wherein:
m is 1-1000;
m′ is 0-1000;
R^{x} and R^{y} are each independently a natural or unnatural amino acid side-chain group, wherein R^{x} and R^{y} are different from each other; and
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group.

Each of the embodiments relating to the R¹, m, m′, R^{x} and R^{y} groups of formula II apply to the R¹, m, m′, R^{x} and R^{y} groups of formula II′ both singly and in combination.

The block poly(amino acid) compounds of the present invention may be further derivatized by a polyethylene glycol group. Such derivatization is well known in the art and is known as PEGylation. In certain embodiments, the PEGylation of a compound of the present invention is achieved by polymerizing ethylene oxide onto the living polymer chain-end. For example, after the desired amino acid monomers are sequentially polymerized onto the compound of formula I, ethylene oxide is then polymerized onto the live polymer end resulting therefrom. Such methods of ethylene oxide polymerization are known in the art and include those described by Kubisa, et al. “Cationic activated monomer polymerization of heterocyclic monomers”/Prog. Polym. Sci./, 1999, 24, 1409-1437.

Alternatively, the compounds of the present invention may be derivatized by a suitable PEG group using PEGylation methods known in the art. Suitable PEG groups are described in detail in U.S. patent application Ser. No. 11/256,735, filed Oct. 25, 2005, the entirety of which is hereby incorporated herein by reference. Accordingly, another embodiment of the present invention provides a compound of formula III:

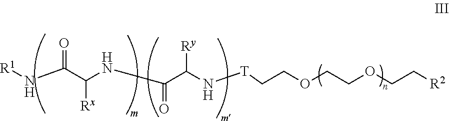

wherein:
n is 10-2500;
m is 1-1000;
m′ is 0-1000;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} and R^{y} are each independently a natural or unnatural amino acid side-chain group, wherein R^{x} and R^{y} are different from each other;
T is a valence bond or a bivalent, saturated or unsaturated, straight or branched C₁₋₁₂ alkylene chain, wherein 0-6 methylene units of Q are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
each R is independently hydrogen or an optionally substituted aliphatic group;
R² is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³;
q and r are each independently 0-4;
each R³ is independently an optionally substituted group selected from aliphatic, a 5-8-membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
   two R³ on the same nitrogen atom are taken together with said nitrogen atom to form an optionally substituted 4-7-membered saturated, partially unsaturated, or aryl ring having 1-4 heteroatoms independently selected from nitrogen, oxygen, or sulfur; and
R⁴ is hydrogen, halogen, N₃, CN, a mono-protected amine, a di-protected amine, a protected aldehyde, a protected hydroxyl, a protected carboxylic acid, a protected thiol, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety.

Each of the embodiments relating to the R¹, m, m′, R^{x} and R^{Y} groups of formula II apply to the R¹, m, m′, R^{x} and R^{y} groups of formula III both singly and in combination.

As defined generally above, the T group of formula III is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur. In certain embodiments, T is a valence bond. In other embodiments, T is a bivalent, saturated C₁₋₁₂ alkylene chain, wherein 0-6 methylene units of T are independently replaced by -Cy-, —O—, —NH—, —S—, —OC(O)—, —C(O)O—, or —C(O)—, wherein -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

In certain embodiments, T is -Cy- (i.e. a C₁ alkylene chain wherein the methylene unit is replaced by -Cy-), wherein -Cy- is an optionally substituted 5-8 membered bivalent, saturated, partially unsaturated, or aryl ring having 0-4 heteroatoms independently selected from nitrogen, oxygen, or sulfur. According to one aspect of the present invention, -Cy- is an optionally substituted bivalent aryl group. According to another aspect of the present invention, -Cy- is an optionally substituted bivalent phenyl group. In other embodiments, -Cy- is an optionally substituted 5-8 membered bivalent, saturated carbocyclic ring. In still other embodiments, -Cy- is an optionally substituted 5-8 membered bivalent, saturated heterocyclic ring having 1-2 heteroatoms independently selected from nitrogen, oxygen, or sulfur. Exemplary -Cy- groups include bivalent rings selected from phenyl, pyridyl, pyrimidinyl, cyclohexyl, cyclopentyl, or cyclopropyl.

In certain embodiments, the T group of formula III is —O—, —S—, —NH—, or —C(O)O—. In other embodiments, the T group of formula III is -Cy-, —C(O)—, —C(O)NH—, or —NHC(O)—. In still other embodiments, the T group of formula III is any of —OCH₂—, —OCH₂C(O)—, —OCH₂CH₂C(O)—, —OCH₂CH₂O—, —OCH₂CH₂S—, —OCH₂CH₂C(O)O—, —OCH₂CH₂NH—, —OCH₂CH₂NHC(O)—, —OCH₂CH₂C(O)NH—, and —NHC(O)CH₂CH₂C(O)O—. According to another aspect, the T group of formula III is any of —OCH₂CH₂NHC(O)CH₂CH₂C(O)O—, —OCH₂CH₂NHC(O)CH₂OCH₂C(O)O—, —OCH₂CH₂NHC(O)CH₂OCH₂C(O)NH—, —CH₂C(O)NH—, —CH₂C(O)NHNH—, or —OCH₂CH₂NHNH—.

Representative T groups are set forth in Table 1, below.

TABLE 1
Representative T groups

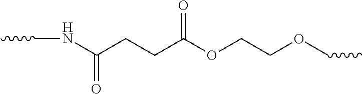

i

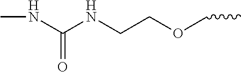

ii

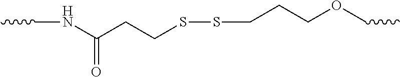

iii

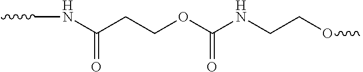

iv

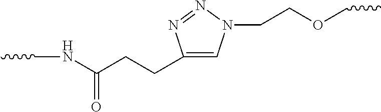

v

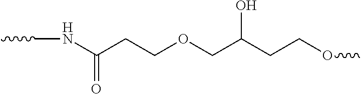

vi

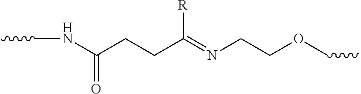

vii

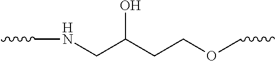

viii

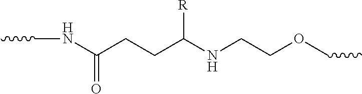

ix

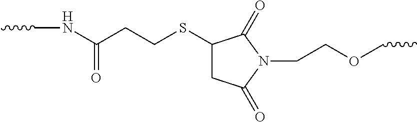

x

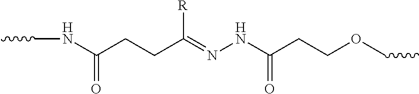

xi

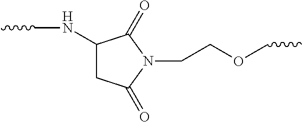

xii

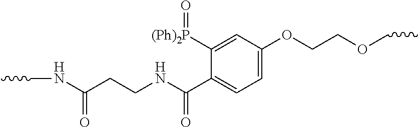

xiii

As defined generally above, the R² group of formula III is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³, wherein q and r are each independently 0-4, each R³ is independently an optionally substituted group selected from aliphatic, a 5-8-membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or two R³ on the same nitrogen atom are taken together with said nitrogen atom to form an optionally substituted 4-7-membered saturated, partially unsaturated, or aryl ring having 1-4 heteroatoms independently selected from nitrogen, oxygen, or sulfur; and R⁴ is hydrogen, halogen, CN, a mono-protected amine, a di-protected amine, a protected aldehyde, a protected hydroxyl, a protected carboxylic acid, a protected thiol, or an optionally substituted group selected from aliphatic, a 5-8-membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety,

In certain embodiments, the R² group of formula III is —N₃.

In other embodiments, the R² group of formula III is —CN.

In other embodiments, the R² group of formula III is —Br, —Cl, —F, or —I.

In certain embodiments, the R² group of formula III is —OS(O)₂R³, wherein R³ is an optionally substituted aliphatic group, or an optionally substituted 5-8-membered aryl ring. Examplary R³ groups include p-tolyl and methyl. In certain embodiments, R² is p-toluenesulfonyloxy or methanesulfonyloxy.

In certain embodiments, the R² group of formula III is —OR³ wherein R³ is an optionally substituted aliphatic group. One exemplary R³ group is 5-norbornen-2-yl-methyl. According to yet another aspect of the present invention, the R² group of formula III is —OR³ wherein R³ is a C₁₋₆ aliphatic group substituted with N₃. Examples include —CH₂N₃. In some embodiments, R³ is an optionally substituted C₁₋₆ alkyl group. Examples include methyl, ethyl, propyl, butyl, pentyl, hexyl, 2-(tetrahydropyran-2-yloxy)ethyl, pyridin-2-yldisulfanylmethyl, methyldisulfanylmethyl, (4-acetylenylphenyl)methyl, 3-(methoxycarbonyl)-prop-2-ynyl, methoxycarbonylmethyl, 2-(N-methyl-N-(4-acetylenylphenyl)carbonylamino)-ethyl, 2-phthalimidoethyl, 4-bromobenzyl, 4-chlorobenzyl, 4-fluorobenzyl, 4-iodobenzyl, 4-propargyloxybenzyl, 2-nitrobenzyl, 4-(bis-4-acetylenylbenzyl)aminomethyl-benzyl, 4-propargyloxy-benzyl, 4-dipropargylamino-benzyl, 4-(2-propargyloxy-ethyldisulfanyl)benzyl, 2-propargyloxy-ethyl, 2-propargyldisulfanyl-ethyl, 4-propargyloxy-butyl, 2-(N-methyl-N-propargylamino)ethyl, and 2-(2-dipropargylaminoethoxy)-ethyl. In other embodiments, R³ is an optionally substituted C₂₋₄ alkenyl group. Examples include vinyl, allyl, crotyl, 2-propenyl, and but-3-enyl. When R³ group is a substituted aliphatic group, suitable substituents on R³ include N₃, CN, and halogen. In certain embodiments, R³ is —CH₂CN, —CH₂CH₂CN, —CH₂CH(OCH₃)₂, 4-(bisbenzyloxymethyl)phenylmethyl, and the like.

According to another aspect of the present invention, the R² group of formula III is —OR³ wherein R³ is an optionally substituted C₂₋₄ alkynyl group. Examples include —CC≡CH, —CH₂C≡CH, —CH₂C≡CCH₃, and —CH₂CH₂≡CH. In certain embodiments, R² is propargyloxy.

In other embodiments, the R² group of formula III is —OC(O)R³ wherein R³ is an optionally substituted aliphatic group. Examples include methyl, ethyl, propyl, butyl, pentyl, hexyl, acetylenyl, propargyl, but-3-ynyl, vinyl, crotyl, 2-propenyl, azidomethyl, 5-norbornen-2-yl, octen-5-yl, triisopropylsilylacetylenyl, 4-vinylphenyl, 4-dipropargylaminophenyl, 4-propargyloxyphenyl, 4-(2-propargyldisulfanyl)methyl-phenyl, and 2-(propargyloxycarbonyl)ethyl.

In certain embodiments, the R² group of formula III is —OR³ wherein R³ is an optionally substituted 5-8-membered aryl ring. In certain embodiments, R³ is optionally substituted phenyl or optionally substituted pyridyl. Examples include phenyl, 4-t-butoxycarbonylaminophenyl, 4-azidomethylphenyl, 4-propargyloxyphenyl, 2-pyridyl, 3-pyridyl, and 4-pyridyl. In certain embodiments, R² is 4-t-butoxycarbonylaminophenoxy, 4-azidomethylphenoxy, or 4-propargyloxyphenoxy.

In certain embodiments, the R² group of formula III is —OR³ wherein R³ is an optionally substituted phenyl ring. Suitable substituents on the R³ phenyl ring include halogen; —(CH₂)₀₋₄R^{∘}; —(CH₂)₀₋₄OR^{∘}; —(CH₂)₀₋₄—CH(OR^{∘)}₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —(CH₂)₀₋₄SC(O)R^{∘}; —(CH₂)₀₋₄C(O)NR^{∘}₂; —C(S)NR^{∘}₂; —CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; SiR^{∘}₃; wherein each independent occurrence of R^{∘} is as defined herein supra. In other embodiments, the R² group of formula III is —OR³ wherein R³ is phenyl substituted with one or more optionally substituted C₁₋₆ aliphatic groups. In still other embodiments, R³ is phenyl substituted with vinyl, allyl, acetylenyl, —CH₂N₃, —CH₂CH₂N₃, —CH₂C≡CH₃, or —CH₂C≡CH.

In certain embodiments, the R² group of formula III is —OR³ wherein R³ is phenyl substituted with N₃, N(R^{∘})₂, CO₂R^{∘}, or C(O)R^{∘} wherein each R^{∘} is independently as defined herein supra.

In other embodiments, the R² group of formula III is a protected hydroxyl group. In certain embodiments the protected hydroxyl of the R² moiety is an ester, carbonate, sulfonate, allyl ether, ether, silyl ether, alkyl ether, arylalkyl ether, or alkoxyalkyl ether. In certain embodiments, the ester is a formate, acetate, proprionate, pentanoate, crotonate, or benzoate. Exemplary esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Exemplary carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Exemplary alkyl ethers include methyl, benzyl, p-methoxybenzyl, 3,4-dimethoxybenzyl, trityl, t-butyl, and allyl ether, or derivatives thereof. Exemplary alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examplary arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

In certain embodiments, the R² group of formula III is —N(R³)₂ wherein each R³ is independently an optionally substituted group selected from aliphatic, phenyl, naphthyl, a 5-6 membered aryl ring having 1-4 heteroatoms independently selected from nitrogen, oxygen, or sulfur, or a 8-10 membered bicyclic aryl ring having 1-5 heteroatoms independently selected from nitrogen, oxygen, or sulfur, or a detectable moiety.

In other embodiments, the R² group of formula III is —N(R³)₂ wherein the two R³ groups are taken together with said nitrogen atom to form an optionally substituted 4-7 membered saturated, partially unsaturated, or aryl ring having 1-4 heteroatoms independently selected from nitrogen, oxygen, or sulfur. According to another embodiment, the two R³ groups are taken together to form a 5-6-membered saturated or partially unsaturated ring having one nitrogen wherein said ring is substituted with one or two oxo groups. Such R² groups include, but are not limited to, phthalimide, maleimide and succinimide.

In certain embodiments, the R² group of formula III is a mono-protected or di-protected amino group. In certain embodiments R² is a mono-protected amine. In certain embodiments R² is a mono-protected amine selected from aralkylamines, carbamates, allyl amines, or amides. Examplary mono-protected amino moieties include t-butyloxycarbonylamino, ethyloxycarbonylamino, methyloxycarbonylamino, trichloroethyloxy-carbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino. In other embodiments R² is a di-protected amine. Exemplary di-protected amino moieties include di-benzylamino, di-allylamino, phthalimide, maleimido, succinimido, pyrrolo, 2,2,5,5-tetramethyl-[1,2,5]azadisilolidino, and azido. In certain embodiments, the R² moiety is phthalimido. In other embodiments, the R² moiety is mono- or di-benzylamino or mono- or di-allylamino.

In other embodiments, the R² group of formula III is a protected aldehyde group. In certain embodiments the protected aldehydro moiety of R² is an acyclic acetal, a cyclic acetal, a hydrazone, or an imine. Exemplary R² groups include dimethyl acetal, diethyl acetal, diisopropyl acetal, dibenzyl acetal, bis(2-nitrobenzyl)acetal, 1,3-dioxane, 1,3-dioxolane, and semicarbazone. In certain embodiments, R² is an acyclic acetal or a cyclic acetal. In other embodiments, R² is a dibenzyl acetal.

In yet other embodiments, the R² group of formula III is a protected carboxylic acid group. In certain embodiments, the protected carboxylic acid moiety of R² is an optionally substituted ester selected from C₁₋₆ aliphatic or aryl, or a silyl ester, an activated ester, an amide, or a hydrazide. Examples of such ester groups include methyl, ethyl, propyl, isopropyl, butyl, isobutyl, benzyl, and phenyl ester. In other embodiments, the protected carboxylic acid moiety of R² is an oxazoline or an ortho ester. Examples of such protected carboxylic acid moieties include oxazolin-2-yl and 2-methoxy-[1,3]dioxin-2-yl.

According to another embodiment, the R² group of formula III is a protected thiol group. In certain embodiments, the protected thiol of R² is a disulfide, thioether, silyl thioether, thioester, thiocarbonate, or a thiocarbamate. Examples of such protected thiols include triisopropylsilyl thioether, t-butyldimethylsilyl thioether, t-butyl thioether, benzyl thioether, p-methylbenzyl thioether, triphenylmethyl thioether, and p-methoxyphenyldiphenylmethyl thioether. In other embodiments, R² is an optionally substituted thioether selected from alkyl, benzyl, or triphenylmethyl, or trichloroethoxycarbonyl thioester. In certain embodiments, R¹ is —S—S-pyridin-2-yl, —S—SBn, —S—SCH₃, or —S—S(p-ethynylbenzyl). In certain embodiments, R¹ is —S—S-pyridin-2-yl.

In still other embodiments, the R² group of formula III is a detectable moiety. According to another aspect of the invention, the R² group of formula III is a fluorescent moiety. Such fluorescent moieties are well known in the art and include coumarins, quinolones, benzoisoquinolones, hostasol, and Rhodamine dyes, to name but a few. Exemplary fluorescent moieties comprising R² include anthracen-9-yl-methoxy, pyren-4-yl-methoxy, 2-(9-H-carbazol-9-yl)-ethoxy, the carboxylate of rhodamine B, and the carboxylate of coumarin 343.

In certain embodiments, the R² group of formula III is a group suitable for Click chemistry. One of ordinary skill in the art would recognize that certain R² groups of the present invention are suitable for Click chemistry.

Compounds of formula III having R² groups suitable for Click chemistry are useful for conjugating said compounds to biological systems such as proteins, viruses, and cells, to name but a few. After conjugation to a biomolecule, drug, cell, substrate, or the like, the other end-group functionality, corresponding to the R¹ moiety of formula III, can be used to attach targeting groups for cell specific delivery including, but not limited to, fluorescent dyes, covalent attachment to surfaces, and incorporation into hydrogels. Thus, another embodiment of the present invention provides a method of conjugating the R² group of a compound of formula III to a macromolecule via Click chemistry. Yet another embodiment of the present invention provides a macromolecule conjugated to a compound of formula III via the R² group.

According to one embodiment, the R² group of formula III is an azide-containing group. According to another embodiment, the R² group of formula III is an alkyne-containing group.

In certain embodiments, the R² group of formula III has a terminal alkyne moiety. In other embodiments, the R² group of formula III is an alkyne-containing moiety having an electron withdrawing group. Accordingly, in such embodiments, the R² group of formula III is

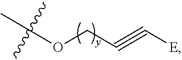

wherein E is an electron withdrawing group and y is 0-6. Such electron withdrawing groups are known to one of ordinary skill in the art. In certain embodiments, E is an ester. In other embodiments, the R² group of formula III is

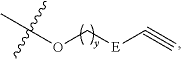

wherein E is an electron withdrawing group, such as a —C(O)O— group and y is 0-6.

According to another aspect, the present invention provides a compound of formula III-a:

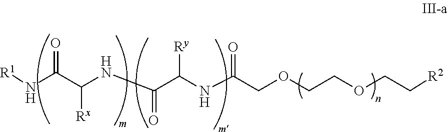

wherein:
n is 10-2500;
m is 1-1000;
m′ is 0-1000;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} and R^{y} are each independently a natural or unnatural amino acid side-chain group, wherein R^{x} and R^{y} are different from each other; and
R² is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³;
q and r are each independently 0-4;
each R³ is independently an optionally substituted group selected from aliphatic, a 5-8-membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or:
   two R³ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur; and
R⁴ is hydrogen, halogen, N₃, CN, a mono-protected amine, a di-protected amine, a protected aldehyde, a protected hydroxyl, a protected carboxylic acid, a protected thiol, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety.

Each of the embodiments relating to the R¹, m, m′, R^{x} and R^{y} groups of formula II apply to the R¹, m, m′, R^{x} and R^{y} groups of formula both singly and in combination.

As defined generally above, the R² group of formula III-a is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³, wherein q and r are each independently 0-4, each R³ is independently an optionally substituted group selected from aliphatic, a 5-8-membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or two R³ on the same nitrogen atom are taken together with said nitrogen atom to form an optionally substituted 4-7-membered saturated, partially unsaturated, or aryl ring having 1-4 heteroatoms independently selected from nitrogen, oxygen, or sulfur; and R⁴ is hydrogen, halogen, CN, a mono-protected amine, a di-protected amine, a protected aldehyde, a protected hydroxyl, a protected carboxylic acid, a protected thiol, or an optionally substituted group selected from aliphatic, a 5-8-membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety,

In certain embodiments, the R² group of formula III-a is —N₃.

In other embodiments, the R² group of formula III-a is —CN.

In other embodiments, the R² group of formula III-a is —Br, —Cl, —F, or —I.

In certain embodiments, the R² group of formula III-a is —OS(O)₂R³, wherein R³ is an optionally substituted aliphatic group, or an optionally substituted 5-8-membered aryl ring. Examplary R³ groups include p-tolyl and methyl. In certain embodiments, R² is p-toluenesulfonyloxy or methanesulfonyloxy.

In certain embodiments, the R² group of formula III-a is —OR³ wherein R³ is an optionally substituted aliphatic group. One exemplary R³ group is 5-norbornen-2-yl-methyl. According to yet another aspect of the present invention, the R² group of formula III-a is —OR³ wherein R³ is a C₁₋₆ aliphatic group substituted with N₃. Examples include —CH₂N3. In some embodiments, R³ is an optionally substituted C₁₋₆ alkyl group. Examples include methyl, ethyl, propyl, butyl, pentyl, hexyl, 2-(tetrahydropyran-2-yloxy)ethyl, pyridin-2-yldisulfanylmethyl, methyldisulfanylmethyl, (4-acetylenylphenyl)methyl, 3-(methoxycarbonyl)-prop-2-ynyl, methoxycarbonylmethyl, 2-(N-methyl-N-(4-acetylenylphenyl)carbonylamino)-ethyl, 2-phthalimidoethyl, 4-bromobenzyl, 4-chlorobenzyl, 4-fluorobenzyl, 4-iodobenzyl, 4-propargyloxybenzyl, 2-nitrobenzyl, 4-(bis-4-acetylenylbenzyl)aminomethyl-benzyl, 4-propargyloxy-benzyl, 4-dipropargylamino-benzyl, 4-(2-propargyloxy-ethyldisulfanyl)benzyl, 2-propargyloxy-ethyl, 2-propargyldisulfanyl-ethyl, 4-propargyloxy-butyl, 2-(N-methyl-N-propargylamino)ethyl, and 2-(2-dipropargylaminoethoxy)-ethyl. In other embodiments, R³ is an optionally substituted C₂₋₆ alkenyl group. Examples include vinyl, allyl, crotyl, 2-propenyl, and but-3-enyl. When R³ group is a substituted aliphatic group, suitable substituents on R³ include N₃, CN, and halogen. In certain embodiments, R³ is —CH₂CN, —CH₂CH₂CN, —CH₂CH(OCH₃)₂, 4-(bisbenzyloxymethyl)phenylmethyl, and the like.

According to another aspect of the present invention, the R² group of formula III-a is —OR³ wherein R³ is an optionally substituted C₂₋₆ alkynyl group. Examples include —CC≡CH, —CH₂C≡CH, —CH₂C≡CCH₃, and —CH₂CH₂C≡CH. In certain embodiments, R² is propargyloxy.

In other embodiments, the R² group of formula III-a is —OC(O)R³ wherein R³ is an optionally substituted aliphatic group. Examples include methyl, ethyl, propyl, butyl, pentyl, hexyl, acetylenyl, propargyl, but-3-ynyl, vinyl, crotyl, 2-propenyl, azidomethyl, 5-norbornen-2-yl, octen-5-yl, triisopropylsilylacetylenyl, 4-vinyiphenyl, 4-dipropargylaminophenyl, 4-propargyloxyphenyl, 4-(2-propargyldsulfanyl)methyl-phenyl, and 2-(propargyloxycarbonyl)ethyl.

In certain embodiments, the R² group of formula III-a is —OR² wherein R² is an optionally substituted 5-8-membered aryl ring. In certain embodiments, R³ is optionally substituted phenyl or optionally substituted pyridyl. Examples include phenyl, 4-t-butoxycarbonylaminophenyl, 4-azidomethylphenyl, 4-propargyloxyphenyl, 2-pyridyl, 3-pyridyl, and 4-pyridyl. In certain embodiments, R² is 4-t-butoxycarbonylaminophenoxy, 4-azidomethylphenoxy, or 4-propargyloxyphenoxy.

In certain embodiments, the R² group of formula III-a is —OR³ wherein R³ is an optionally substituted phenyl ring. Suitable substituents on the R³ phenyl ring include halogen; —(CH₂)₀₋₄R^{∘}; —(CH₂)₀₋₄OR^{∘}; —(CH₂)₀₋₄CH(OR^{∘)}₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N)(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —(CH₂)₀₋₄SC(O)R^{∘}; —(CH₂)₀₋₄C(O)NR^{∘}₂; —C(S)NR^{∘}₂; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; SiR^{∘}₃; wherein each independent occurrence of R^{∘} is as defined herein supra. In other embodiments, the R² group of formula III-a is —OR³ wherein R³ is phenyl substituted with one or more optionally substituted C₁₋₆ aliphatic groups. In still other embodiments, R³ is phenyl substituted with vinyl, allyl, acetylenyl, —CH₂N₃, —CH₂CH₂N₃, —CH₂C≡CCH₃, or —CH₂C≡CH.

In certain embodiments, the R² group of formula III-a is —OR³ wherein R³ is phenyl substituted with N₃, N(R^{∘})₂, CO₂R^{∘}, or C(O)R^{∘} wherein each R^{∘} is independently as defined herein supra.

In other embodiments, the R² group of formula III-a is a protected hydroxyl group. In certain embodiments the protected hydroxyl of the R² moiety is an ester, carbonate, sulfonate, allyl ether, ether, silyl ether, alkyl ether, arylalkyl ether, or alkoxyalkyl ether. In certain embodiments, the ester is a formate, acetate, proprionate, pentanoate, crotonate, or benzoate. Exemplary esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Exemplary carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Exemplary alkyl ethers include methyl, benzyl, p-methoxybenzyl, 3,4-dimethoxybenzyl, trityl, t-butyl, and allyl ether, or derivatives thereof. Exemplary alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examplary arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

In certain embodiments, the R² group of formula III-a is —N(R³)₂ wherein each R³ is independently an optionally substituted group selected from aliphatic, phenyl, naphthyl, a 5-6 membered aryl ring having 1-4 heteroatoms independently selected from nitrogen, oxygen, or sulfur, or a 8-10 membered bicyclic aryl ring having 1-5 heteroatoms independently selected from nitrogen, oxygen, or sulfur, or a detectable moiety.

In other embodiments, the R² group of formula III-a is —N(R³)₂ wherein the two R³ groups are taken together with said nitrogen atom to form an optionally substituted 4-7 membered saturated, partially unsaturated, or aryl ring having 1-4 heteroatoms independently selected from nitrogen, oxygen, or sulfur. According to another embodiment, the two R³ groups are taken together to form a 5-6-membered saturated or partially unsaturated ring having one nitrogen wherein said ring is substituted with one or two oxo groups. Such R² groups include, but are not limited to, phthalimide, maleimide and succinimide.

In certain embodiments, the R² group of formula III-a is a mono-protected or di-protected amino group. In certain embodiments R² is a mono-protected amine. In certain embodiments R² is a mono-protected amine selected from aralkylamines, carbamates, allyl amines, or amides. Examplary mono-protected amino moieties include t-butyloxycarbonylamino, ethyloxycarbonylamino, methyloxycarbonylamino, trichloroethyloxy-carbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino. In other embodiments R² is a di-protected amine. Exemplary di-protected amino moieties include di-benzylamino, di-allylamino, phthalimide, maleimido, succinimido, pyrrolo, 2,2,5,5-tetramethyl-[1,2,5]azadisilolidino, and azido. In certain embodiments, the R² moiety is phthalimido. In other embodiments, the R² moiety is mono- or di-benzylamino or mono- or di-allylamino.

In other embodiments, the R² group of formula III-a is a protected aldehyde group. In certain embodiments the protected aldehydro moiety of R² is an acyclic acetal, a cyclic acetal, a hydrazone, or an imine. Exemplary R² groups include dimethyl acetal, diethyl acetal, diisopropyl acetal, dibenzyl acetal, bis(2-nitrobenzyl)acetal, 1,3-dioxane, 1,3-dioxolane, and semicarbazone. In certain embodiments, R² is an acyclic acetal or a cyclic acetal. In other embodiments, R² is a dibenzyl acetal.

In yet other embodiments, the R² group of formula III-a is a protected carboxylic acid group. In certain embodiments, the protected carboxylic acid moiety of R² is an optionally substituted ester selected from C₁₋₆ aliphatic or aryl, or a silyl ester, an activated ester, an amide, or a hydrazide. Examples of such ester groups include methyl, ethyl, propyl, isopropyl, butyl, isobutyl, benzyl, and phenyl ester. In other embodiments, the protected carboxylic acid moiety of R² is an oxazoline or an ortho ester. Examples of such protected carboxylic acid moieties include oxazolin-2-yl and 2-methoxy-[1,3]dioxin-2-yl.

According to another embodiment, the R² group of formula III-a is a protected thiol group. In certain embodiments, the protected thiol of R² is a disulfide, thioether, silyl thioether, thioester, thiocarbonate, or a thiocarbamate. Examples of such protected thiols include triisopropylsilyl thioether, t-butyldimethylsilyl thioether, t-butyl thioether, benzyl thioether, p-methylbenzyl thioether, triphenylmethyl thioether, and p-methoxyphenyldiphenylmethyl thioether. In other embodiments, R² is an optionally substituted thioether selected from alkyl, benzyl, or triphenylmethyl, or trichloroethoxycarbonyl thioester. In certain embodiments, R¹ is —S—S-pyridin-2-yl, —S—SBn, —S—SCH₃, or —S—S(p-ethynylbenzyl). In certain embodiments, R¹ is —S—S-pyridin-2-yl.

In still other embodiments, the R² group of formula III-a is a detectable moiety. According to another aspect of the invention, the R² group of formula III-a is a fluorescent moiety. Such fluorescent moieties are well known in the art and include coumarins, quinolones, benzoisoquinolones, hostasol, and Rhodamine dyes, to name but a few. Exemplary fluorescent moieties comprising R² include anthracen-9-yl-methoxy, pyren-4-yl-methoxy, 2-(9-H-carbazol-9-yl)-ethoxy, the carboxylate of rhodamine B, and the carboxylate of coumarin 343.

In certain embodiments, the R² group of formula III-a is a group suitable for Click chemistry. One of ordinary skill in the art would recognize that certain R² groups of the present invention are suitable for Click chemistry.

Compounds of formula III-a having R² groups suitable for Click chemistry are useful for conjugating said compounds to biological systems such as proteins, viruses, and cells, to name but a few. After conjugation to a biomolecule, drug, cell, substrate, or the like, the other end-group functionality, corresponding to the R¹ moiety of formula III-a, can be used to attach targeting groups for cell specific delivery including, but not limited to, fluorescent dyes, covalent attachment to surfaces, and incorporation into hydrogels. Thus, another embodiment of the present invention provides a method of conjugating the R² group of a compound of formula III-a to a macromolecule via Click chemistry. Yet another embodiment of the present invention provides a macromolecule conjugated to a compound of formula III-a via the R² group.

According to one embodiment, the R² group of formula III-a is an azide-containing group. According to another embodiment, the R² group of formula III-a is an alkyne-containing group.

In certain embodiments, the R² group of formula III-a has a terminal alkyne moiety. In other embodiments, the R² group of formula III-a is an alkyne-containing moiety having an electron withdrawing group. Accordingly, in such embodiments, the R² group of formula III-a is

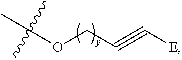

wherein E is an electron withdrawing group and y is 0-6. Such electron withdrawing groups are known to one of ordinary skill in the art. In certain embodiments, E is an ester. In other embodiments, the R² group of formula III-a is

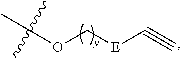

wherein E is an electron withdrawing group, such as a —C(O)O— group and y is 0-6.

Exemplary R² groups are set forth in Table 2, below.

TABLE 2
Representative R² groups

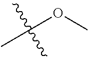

i

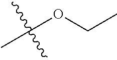

ii

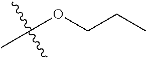

iii

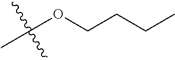

iv

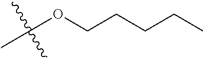

v

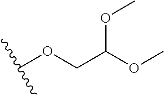

vi

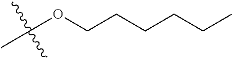

vii

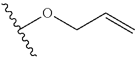

viii

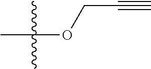

ix

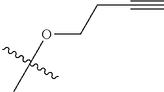

x

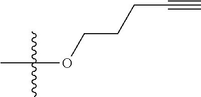

xi

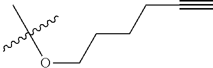

xii

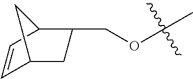

xiii

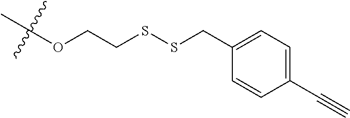

xiv

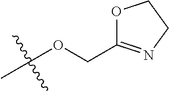

xv

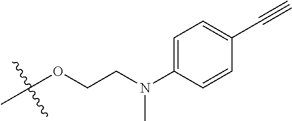

xvi

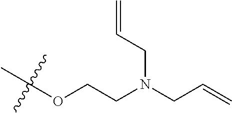

xvii

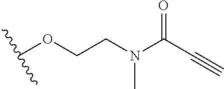

xviii

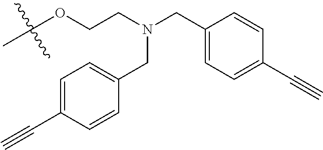

xix

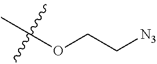

xx

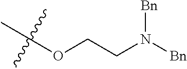

xxi

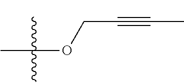

xxii

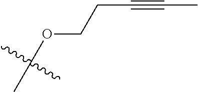

xxiii

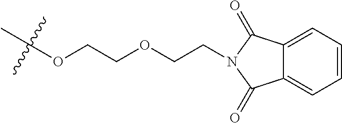

xxiv

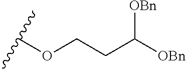

xxv

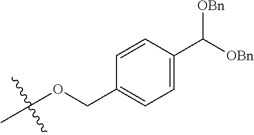

xvi

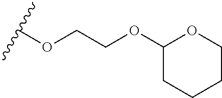

xvii

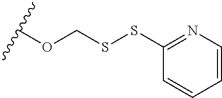

xviii

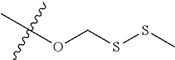

xxix

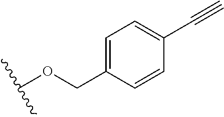

xxx

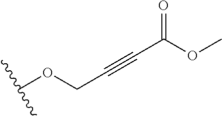

xxxi

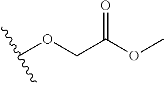

xxxii

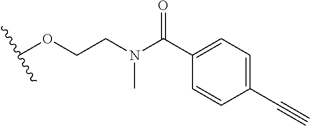

xxxiii

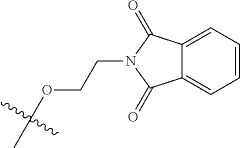

xxxiv

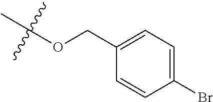

xxxv

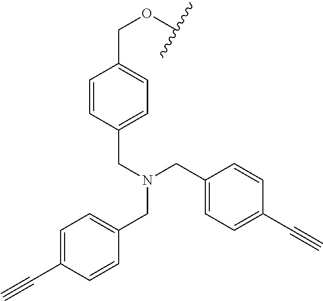

xxxvi

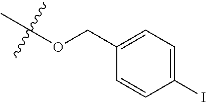

xxxvii

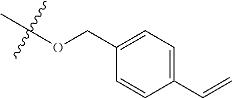

xxxviii

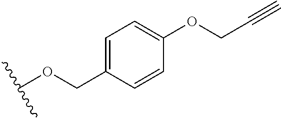

xxxix

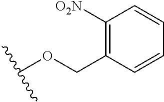

xl

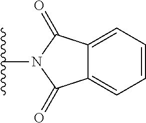

xlii

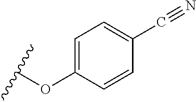

xlii

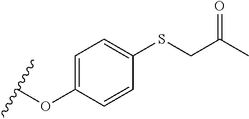

xliii

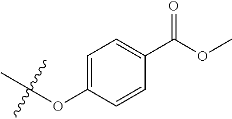

xliv

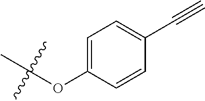

xlv

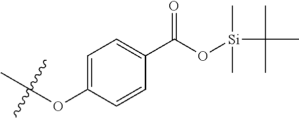

xlviv

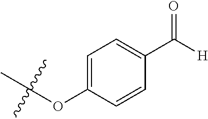

xlvii

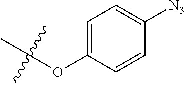

xlviii

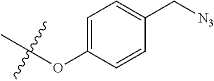

xlix

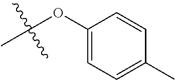

l

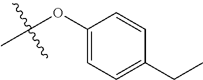

li

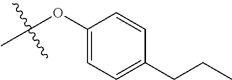

lii

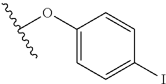

liii

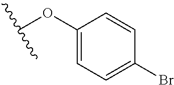

liv

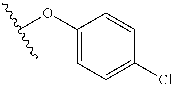

lv

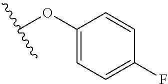

lvi

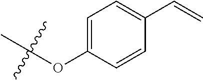

lvii

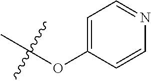

lviii

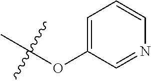

lix

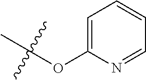

lx

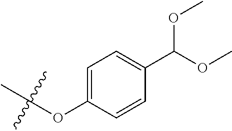

lxi

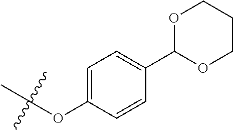

lxii

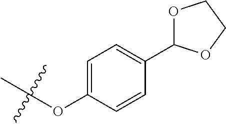

lxiii

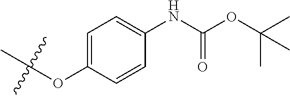

lxiv

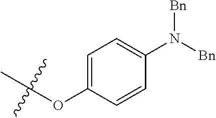

lxi

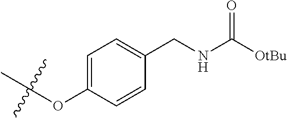

lxii

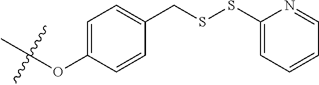

lxiii

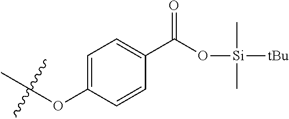

lxiv

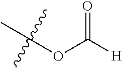

lxv

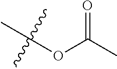

lxvi

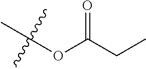

lxvii

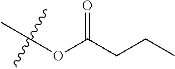

lxviii

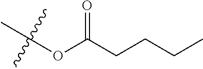

lxix

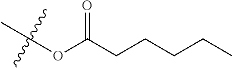

lxx

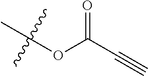

lxxi

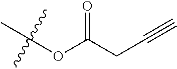

lxxii

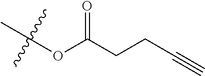

lxxiii

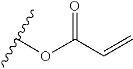

lxxiv

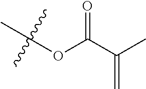

lxxv

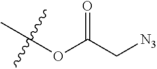

lxxvi

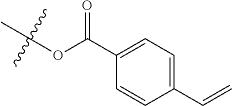

lxxvii

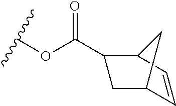

lxxviii

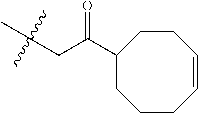

lxxix

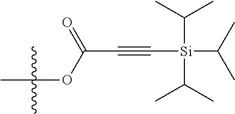

lxxx

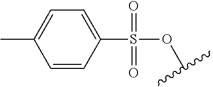

lxxxi

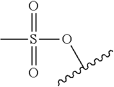

lxxxii

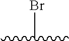

lxxxiii

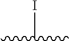

lxxxiv

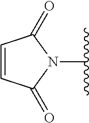

lxxxv

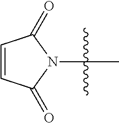

lxxxvi

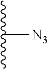

lxxxvii

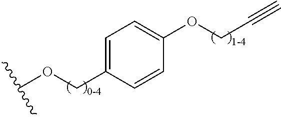

lxxxviii

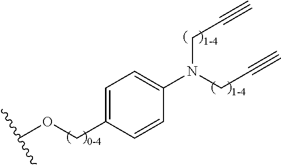

lxxxix

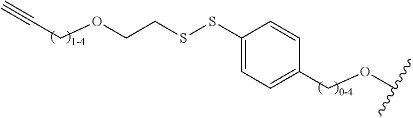

xc

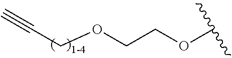

xci

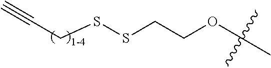

xcii

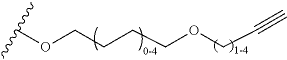

xciii

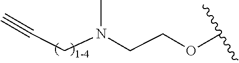

xciv

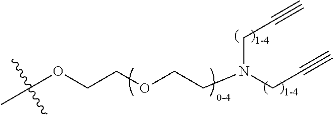

xcv

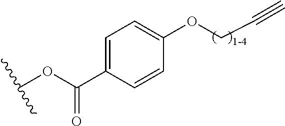

xcvi

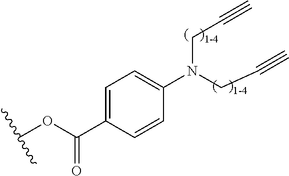

xcvii

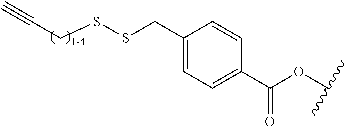

xcviii

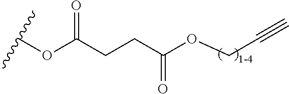

xcix

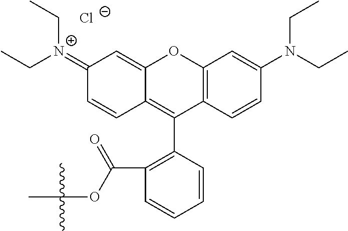

c

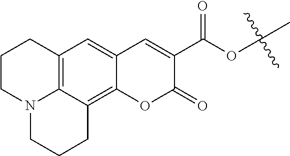

ci

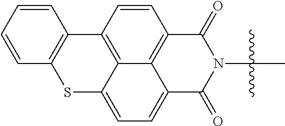

cii

In certain embodiments, the R² group of formula III is selected from any of those R² groups depicted in Table 2, supra. In other embodiments, the R² group of formula III is group xlii or xxiv. In yet other embodiments, the R² group of formula III is xix, xvii, xviii, xxix, xxxii, xlviv, xlvii, or xlviii.

According to another aspect of the present invention, the R² group of formula III is ix, xxii, xxx, xxxi, xlv, xlviii, xlix, lxxi.

Yet another aspect of the present invention provides a compound of formula III-a:

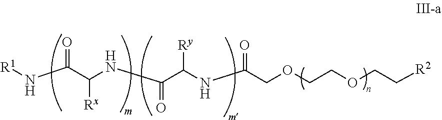

wherein:
n is 10-2500;
m is 1-1000;
m′ is 0-1000;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} and R^{y} are each independently a natural or unnatural amino acid side-chain group, wherein R^{x} and R^{y} are different from each other; and
R² is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³;
q and r are each independently 0-4;
each R³ is independently an optionally substituted group selected from aliphatic, a 5-8-membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
   two R³ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur; and
R⁴ is hydrogen, halogen, N₃, CN, a mono-protected amine, a di-protected amine, a protected aldehyde, a protected hydroxyl, a protected carboxylic acid, a protected thiol, or an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
   wherein said compound is prepared by a method comprising the steps of:
(a) providing a compound of formula I:

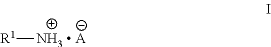

wherein:
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and
A is a suitable acid anion,
(b) polymerizing a first cyclic amino acid monomer onto the amine salt terminal end of formula I, wherein said first cyclic amino acid monomer comprises R^{x};
(c) optionally polymerizing a second cyclic amino acid monomer, comprising R^{y}, onto the living polymer end, wherein said second cyclic amino acid monomer is different from said first cyclic amino acid monomer; and
(d) coupling onto the amine terminus a compound of formula

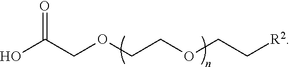

The coupling step (d), as described generally above, is achieved using coupling methods well known in the art. Such methods include those taught in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001. In certain embodiments, the coupling step (d), as described generally above, is achieved by Mitsunobu coupling. In other embodiments, the coupling step (d), as described generally above, is achieved by carbodiimide coupling, using, for example, EDC, DCC, or DIC.

It will be appreciated by one of ordinary in the art that a compound of formula II′ may be further derivatized by treatment of that compound with a suitable terminating agent. Thus, another embodiment of the present invention provides a compound of formula II-a:

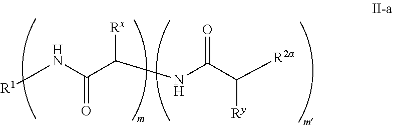

wherein:
m is 1-1000;
m′ is 0-1000;
R^{x} and R^{y} are each independently a natural or unnatural amino acid side-chain group, wherein R^{x} and R^{y} are different from each other; and
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{2a} is a mono-protected amine, a di-protected amine, —NHR³, —N(R³)₂, —NHC(O)R³, —NR³C(O)R³, —NHC(O)NHR³, —NHC(O)N(R³)₂, —NR³C(O)NHR³, —NR³C(O)N(R³)₂, —NHC(O)OR³, —NR³C(O)OR³, —NHSO₂R³, or —NR³SO₂R³; and
   each R³ is independently an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or:
   two R³ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

Each of the embodiments relating to the R¹, m, m′, R^{Y} and R^{X} groups of formula II apply to the R¹, m, m′, R^{Y} and R^{X} groups of formula II-a both singly and in combination.

As defined generally above, the R^{2a} group of formula II-a is a mono-protected amine, a di-protected amine, —NHR³, —N(R³)₂, —NHC(O)R³, —NR³C(O)R³, —NHC(O)NHR³, —NHC(O)N(R³)₂, —NR³C(O)NHR³, —NR³C(O)N(R³)₂, —NHC(O)OR³, —NR³C(O)OR³, —NHSO₂R³, or —NR³SO₂R³, wherein each R³ is independently an optionally substituted group selected from aliphatic, a 5-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or two R³ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

In certain embodiments, the R^{2a} group of formula II-a is —NHR³ or —N(R³)₂ wherein each R³ is an optionally substituted aliphatic group. One exemplary R³ group is 5-norbornen-2-yl-methyl. According to yet another aspect of the present invention, the group of formula II-a is —NHR³ wherein R³ is a C₁₋₆ aliphatic group substituted with N₃. Examples include —CH₂N₃. In some embodiments, R³ is an optionally substituted C₁₋₆ alkyl group. Examples include methyl, ethyl, propyl, butyl, pentyl, hexyl, 2-(tetrahydropyran-2-yloxy)ethyl, pyridin-2-yldisulfanylmethyl, methyldisulfanylmethyl, (4-acetylenylphenyl)methyl, 3-(methoxycarbonyl)-prop-2-ynyl, methoxycarbonylmethyl, 2-(N-methyl-N-(4-acetylenylphenyl)carbonylamino)-ethyl, 2-phthalimidoethyl, 4-bromobenzyl, 4-chlorobenzyl, 4-fluorobenzyl, 4-iodobenzyl, 4-propargyloxybenzyl, 2-nitrobenzyl, 4-(bis-4-acetylenylbenzyl)aminomethyl-benzyl, 4-propargyloxy-benzyl, 4-dipropargylamino-benzyl, 4-(2-propargyloxy-ethyldisulfanyl)benzyl, 2-propargyloxy-ethyl, 2-propargyldisulfanyl-ethyl, 4-propargyloxy-butyl, 2-(N-methyl-N-propargylamino)ethyl, and 2-(2-dipropargylaminoethoxy)-ethyl. In other embodiments, R³ is an optionally substituted C₂₋₄ alkenyl group. Examples include vinyl, allyl, crotyl, 2-propenyl, and but-3-enyl. When R³ group is a substituted aliphatic group, suitable substituents on R³ include N₃, CN, and halogen. In certain embodiments, R³ is —CH₂CN, —CH₂CH₂CN, —CH₂CH(OCH₃)₂, 4-(bisbenzyloxymethyl)phenylmethyl, and the like.

According to another aspect of the present invention, the R^{ea} group of formula II-a is —NHR³ wherein R³ is an optionally substituted C₂₋₄ alkynyl group. Examples include —CC≡CH, —CH₂C≡CH, —CH₂C≡CCH₃, and —CH₂CH₂C≡CH.

In certain embodiments, the R^{2a} group of formula II-a is —NHR³ wherein R³ is an optionally substituted 5-8-membered aryl ring. In certain embodiments, R³ is optionally substituted phenyl or optionally substituted pyridyl. Examples include phenyl, 4-t-butoxycarbonylaminophenyl, 4-azidomethylphenyl, 4-propargyloxyphenyl, 2-pyridyl, 3-pyridyl, and 4-pyridyl. In certain embodiments, R^{2a} is 4-t-butoxycarbonylaminophenylamino, 4-azidomethylphenamino, or 4-propargyloxyphenylamino.

In certain embodiments, the R^{2a} group of formula II-a is —NHR³ wherein R³ is an optionally substituted phenyl ring. Suitable substituents on the R³ phenyl ring include halogen; —(CH₂)₀₋₄R^{∘}; —(CH₂)₀₋₄OR^{∘}; —(CH₂)₀₋₄CH(OR^{∘)}₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —(CH₂)₀₋₄SC(O)R^{∘}; —(CH₂)₀₋₄C(O)NR^{∘}₂; —C(S)NR^{∘}₂; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; SiR^{∘}₃; wherein each independent occurrence of R^{∘} is as defined herein supra. In other embodiments, the R^{2a} group of formula II-a is —NHR³ wherein R³ is phenyl substituted with one or more optionally substituted C₁₋₆ aliphatic groups. In still other embodiments, R³ is phenyl substituted with vinyl, allyl, acetylenyl, —CH₂N₃, —CH₂CH₂N₃, —CH₂C≡CCH₃, or —CH₂C≡CH.

In certain embodiments, the R^{2a} group of formula II-a is —NHR³ wherein R³ is phenyl substituted with N₃, N(R^{∘})₂, CO₂R^{∘}, or C(O)R^{∘} wherein each R^{∘} is independently as defined herein supra.

In certain embodiments, the R^{2a} group of formula II-a is —N(R³)₂ wherein each R³ is independently an optionally substituted group selected from aliphatic, phenyl, naphthyl, a 5-6 membered aryl ring having 1-4 heteroatoms independently selected from nitrogen, oxygen, or sulfur, or a 8-10 membered bicyclic aryl ring having 1-5 heteroatoms independently selected from nitrogen, oxygen, or sulfur, or a detectable moiety.

In other embodiments, the R^{2a} group of formula II-a is —N(R³)₂ wherein the two R³ groups are taken together with said nitrogen atom to form an optionally substituted 4-7 membered saturated, partially unsaturated, or aryl ring having 1-4 heteroatoms independently selected from nitrogen, oxygen, or sulfur. According to another embodiment, the two R³ groups are taken together to form a 5-6-membered saturated or partially unsaturated ring having one nitrogen wherein said ring is substituted with one or two oxo groups. Such R^{2a} groups include, but are not limited to, phthalimide, maleimide and succinimide.

In certain embodiments, the R^{2a} group of formula II-a is a mono-protected or di-protected amino group. In certain embodiments R^{2a} is a mono-protected amine. In certain embodiments R^{2a} is a mono-protected amine selected from aralkylamines, carbamates, allyl amines, or amides. Examplary mono-protected amino moieties include t-butyloxycarbonylamino, ethyloxycarbonylamino, methyloxycarbonylamino, trichloroethyloxy-carbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino. In other embodiments R^{2a} is a di-protected amine. Exemplary di-protected amino moieties include di-benzylamino, di-allylamino, phthalimide, maleimido, succinimido, pyrrolo, 2,2,5,5-tetramethyl-[1,2,5]azadisilolidino, and azido. In certain embodiments, the R^{2a} moiety is phthalimido. In other embodiments, the R^{2a} moiety is mono- or di-benzylamino or mono- or di-allylamino.

In certain embodiments, the R^{2a} group of formula II-a comprises a group suitable for Click chemistry. One of ordinary skill in the art would recognize that certain R^{2a} groups of the present invention are suitable for Click chemistry.

Compounds of formula II-a having R^{2a} groups comprising groups suitable for Click chemistry are useful for conjugating said compounds to biological systems such as proteins, viruses, and cells, to name but a few. After conjugation to a biomolecule, drug, cell, substrate, or the like, the other end-group functionality, corresponding to the R¹ moiety of formula II-a, can be used to attach targeting groups for cell specific delivery including, but not limited to, fluorescent dyes, covalent attachment to surfaces, and incorporation into hydrogels. Thus, another embodiment of the present invention provides a method of conjugating the R^{2a} group of a compound of formula II-a to a macromolecule via Click chemistry. Yet another embodiment of the present invention provides a macromolecule conjugated to a compound of formula II-a via the R^{2a} group.

According to one embodiment, the R^{2a} group of formula II-a is an azide-containing group. According to another embodiment, the R^{2a} group of formula II-a is an alkyne-containing group.

In certain embodiments, the R^{2a} group of formula II-a has a terminal alkyne moiety. In other embodiments, the R^{2a} group of formula II-a is an alkyne-containing moiety having an electron withdrawing group. Accordingly, in such embodiments, the R^{2a} group of formula II-a is

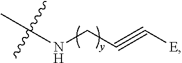

wherein E is an electron withdrawing group and y is 0-6. Such electron withdrawing groups are known to one of ordinary skill in the art. In certain embodiments, E is an ester. In other embodiments, the R^{2a} group of formula II-a is

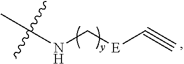

wherein E is an electron withdrawing group, such as a —C(O)O— group and y is 0-6.

According to another embodiment, the present invention provides compounds of formula II-a, as described above, wherein said compounds have a polydispersity index (“PDI”) of about 1.0 to about 1.2. According to another embodiment, the present invention provides compounds of formula II-a, as described above, wherein said compound has a polydispersity index (“PDI”) of about 1.03 to about 1.15. According to yet another embodiment, the present invention provides compounds of formula II-a, as described above, wherein said compound has a polydispersity index (“PDI”) of about 1.10 to about 1.12. According to other embodiments, the present invention provides compounds of formula II-a having a PDI of less than about 1.10.

In certain embodiments, the present invention provides compounds of formula II-a, as described above, wherein n is about 225. In other embodiments, n is about 200 to about 300. In still other embodiments, n is about 200 to about 250. In still other embodiments, n is about 100 to about 150. In still other embodiments, n is about 400 to about 500.

Exemplary R^{2a} groups are set forth in Table 3, below.

TABLE 3
Representative R^{2a} Groups

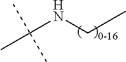

i

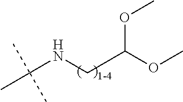

ii

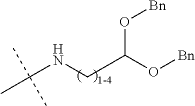

iii

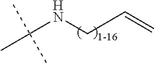

iv

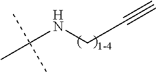

v

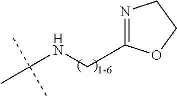

vi

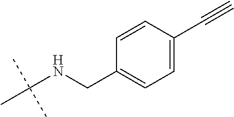

vii

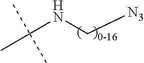

viii

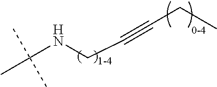

ix

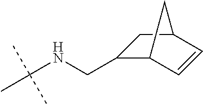

x

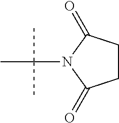

x

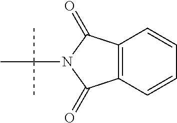

xi

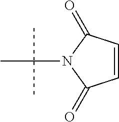

xii

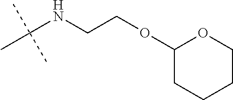

xiii

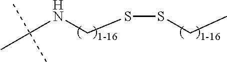

xiv

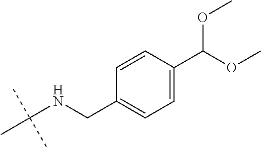

xv

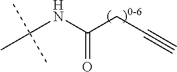

xvi

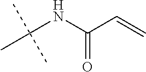

xvii

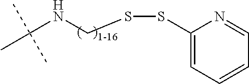

xviii

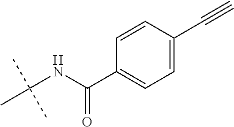

xix

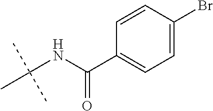

xx

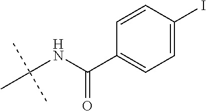

xxi

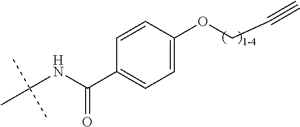

xxii

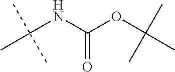

xxiii

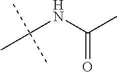

xxiv

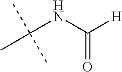

xxv

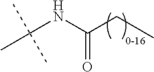

xxvi

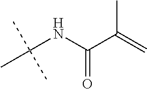

xxvii

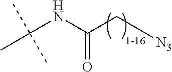

xxviii

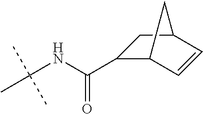

xxix

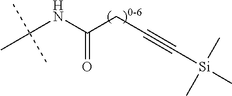

xxx

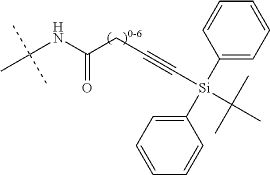

xxxi

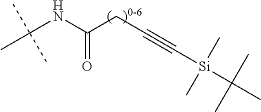

xxxii

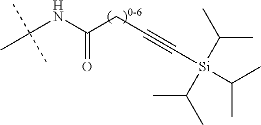

xxxiii

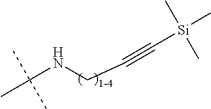

xxxiv

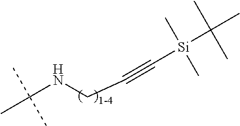

xxxv

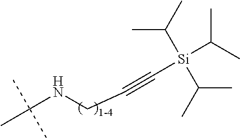

xxxvi

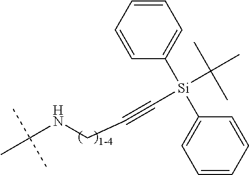

xxxvii

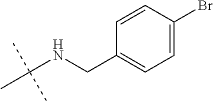

xxxviii

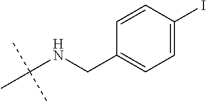

xxxix

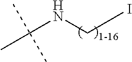

xl

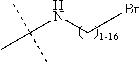

xli

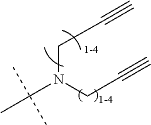

xlii

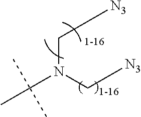

xliii

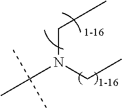

xliv

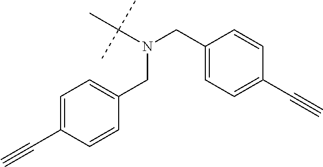

xlv

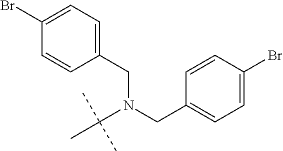

xlvi

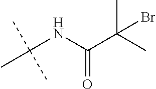

xlvii

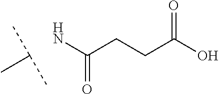

xlviii

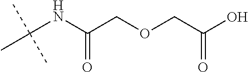

xlvix

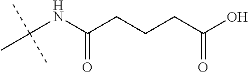

xlx

In certain embodiments, the R^{2a} group of formula II-a is selected from any of those R^{2a} groups depicted in Table 3, supra. In other embodiments, the R^{2a} group of formula II-a is group v, viii, xvi, xix, xxii, xxx, xxxi, xxxii, xxxiii, xxxiv, xtxw, xxxvi, xxxvii, or xlii. In yet other embodiments, the R^{2a} group of formula II-a is xv, xviii, xx, xxi, xxxviii, or xxxix.

It will be appreciated that the non-polymeric amine salt initiators of the present invention may comprise more than one amine salt, and thus the present invention also encompasses bifunctional compound of formula I-a:

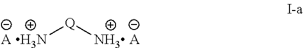

wherein:
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and
each A is a suitable acid anion.

Compounds of formula I-a are useful for preparing poly(amino acid) block copolymers of formula IV:

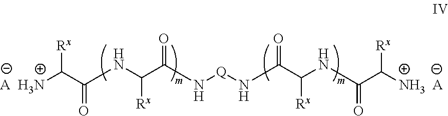

wherein:
m is 1-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} is a natural or unnatural amino acid side-chain group; and
A is a suitable acid anion.

Accordingly, another aspect of the present invention provides a compound of formula IV:

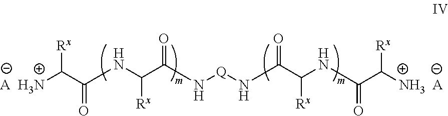

wherein:
m is 1-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} is a natural or unnatural amino acid side-chain group; and
each A is a suitable acid anion.

Each of the embodiments relating to the m and R^{X} groups of formula II apply to the m and R^{X} groups of formula IV both singly and in combination.

In certain embodiments, the Q group of formulae I-a and IV is substituted with —N₃.

In certain embodiments, the Q group of formulae I-a and IV is an optionally substituted bivalent aliphatic group. In some embodiments, said Q moiety is an optionally substituted bivalent alkyl group. In other embodiments, said Q moiety is an optionally substituted bivalent alkynyl or alkenyl group. When said Q moiety is a substituted bivalent aliphatic group, suitable substituents on Q include CN, a mono-protected amino group, a di-protected amino group, a protected aldehyde group, a protected hydroxyl group, a protected carboxylic acid group, a protected thiol group, or a detectable moiety.

In certain embodiments, Q group of formulae I-a and IV is an optionally substituted bivalent group selected from 5-7 membered saturated, partially unsaturated, or aryl monocyclic ring having 0-2 heteroatoms independently selected from oxygen, nitrogen, or sulfur. In other embodiments, R¹ is a 9-10 membered saturated, partially unsaturated, or aryl bicyclic ring having 0-3 heteroatoms independently selected from oxygen, nitrogen, or sulfur. In still other embodiments, Q is a 13-14 membered saturated, partially unsaturated, or aryl tricyclic ring having 0-3 heteroatoms independently selected from oxygen, nitrogen, or sulfur. Such cyclic Q groups, as defined herein, include optionally substituted phenyl, naphthyl, and anthracenyl groups.

In certain embodiments, the Q group of formulae I-a and IV is an optionally substituted 5-6 membered saturated, partially unsaturated, or aryl monocyclic ring having 0-3 heteroatoms independently selected from oxygen, nitrogen, or sulfur. In other embodiments, the Q group is an optionally substituted phenyl group. Exemplary substituents on Q include —N₃, —CN, an amino group, a mono-protected amino group, a di-protected amino group, a protected aldehyde group, a protected hydroxyl group, a protected carboxylic acid group, a protected thiol group, an optionally substituted aliphatic group, or a detectable moiety.

In certain embodiments, the Q group of formulae I-a and IV comprises a fluorescent moiety.

In other embodiments, the Q group of formulae I-a and IV is substituted with a protected hydroxyl group. In certain embodiments the protected hydroxyl of the Q moiety is an ester, carbonate, sulfonate, allyl ether, ether, silyl ether, alkyl ether, arylalkyl ether, or alkoxyalkyl ether. In certain embodiments, the ester is a formate, acetate, proprionate, pentanoate, crotonate, or benzoate. Exemplary esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Exemplary carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Exemplary alkyl ethers include methyl, benzyl, p-methoxybenzyl, 3,4-dimethoxybenzyl, trityl, t-butyl, and allyl ether, or derivatives thereof. Exemplary alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examplary arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

In certain embodiments, the Q group of formulae I-a and IV is substituted with a mono-protected or di-protected amino group. In certain embodiments the amino moiety is a mono-protected amine. In certain embodiments the amino moiety is a mono-protected amine selected from aralkylamines, carbamates, allyl amines, or amides. Examplary mono-protected amino moieties include t-butyloxycarbonylamino, ethyloxycarbonylamino, methyloxycarbonylamino, trichloroethyloxy-carbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino. In other embodiments the amino moiety is a di-protected amine. Exemplary di-protected amines include di-benzylamine, di-allylamine, phthalimide, maleimide, succinimide, pyrrole, 2,2,5,5-tetramethyl-[1,2,5]azadisilolidine, and azide. In certain embodiments, the amino moiety is phthalimido. In other embodiments, the amino moiety is mono- or di-benzylamino or mono- or di-allylamino. In certain embodiments, the amino moiety group is 2-dibenzylaminoethoxy.

In other embodiments, the Q group of formulae I-a and IV is substituted with a protected aldehyde group. In certain embodiments the protected aldehydro moiety of Q is an acyclic acetal, a cyclic acetal, a hydrazone, or an imine. Exemplary protected aldehyde moieties include dimethyl acetal, diethyl acetal, diisopropyl acetal, dibenzyl acetal, bis(2-nitrobenzyl)acetal, 1,3-dioxane, 1,3-dioxolane, and semicarbazone. In certain embodiments, the protected aldehyde moiety is an acyclic acetal or a cyclic acetal. In other embodiments, the protected aldehyde moiety is a dibenzyl acetal.

In yet other embodiments, the Q group of formulae I-a and IV is substituted with a protected carboxylic acid group. In certain embodiments, the protected carboxylic acid moiety of Q is an optionally substituted ester selected from C₁₋₆ aliphatic or aryl, or a silyl ester, an activated ester, an amide, or a hydrazide. Examples of such ester groups include methyl, ethyl, propyl, isopropyl, butyl, isobutyl, benzyl, and phenyl ester. In other embodiments, the protected carboxylic acid moiety of Q is an oxazoline or an ortho ester. Examples of such protected carboxylic acid moieties include oxazolin-2-yl and 2-methoxy-[1,3]dioxin-2-yl. In certain embodiments, the protected carboxylic acid moiety of Q is oxazolin-2-ylmethoxy or 2-oxazolin-2-yl-1-propoxy.

According to another embodiment, the Q group of formulae I-a and IV is substituted with a protected thiol group. In certain embodiments, the protected thiol of Q is a disulfide, thioether, silyl thioether, thioester, thiocarbonate, or a thiocarbamate. Examples of such protected thiols include triisopropylsilyl thioether, t-butyldimethylsilyl thioether, t-butyl thioether, benzyl thioether, p-methylbenzyl thioether, triphenylmethyl thioether, and p-methoxyphenyldiphenylmethyl thioether. In other embodiments, the protected thiol moiety of Q is an optionally substituted thioether selected from alkyl, benzyl, or triphenylmethyl, or trichloroethoxycarbonyl thioester. In certain embodiments, the protected thiol moiety of Q is —S—S-pyridin-2-yl, —S-SBn, —S—SCH₃, or —S—S(p-ethynylbenzyl). In other embodiments, the protected thiol moiety of Q is —S—S-pyridin-2-yl. In still other embodiments, the protected thiol moiety of Q is 2-triphenylmethylsulfanyl-ethoxy.

In other embodiments, the Q group of formulae I-a and IV is substituted with a crown ether moiety. Exemplary crown ether moieties include radicals of 12-crown-4,15-crown-5, and 18-crown-6.

In still other embodiments, the Q group of formulae I-a and IV is substituted with a detectable moiety. According to one aspect of the invention, the Q group of formulae I-a and IV is substituted with a fluorescent moiety. Such fluorescent moieties are well known in the art and include coumarins, quinolones, benzoisoquinolones, hostasol, and Rhodamine dyes, to name but a few. Exemplary fluorescent moieties include anthracen-9-yl, pyren-4-yl, 9-H-carbazol-9-yl, the carboxylate of rhodamine B, and the carboxylate of coumarin 343. In other embodiments, the Q group of formulae I-a and IV is a bivalent fluorescent moiety.

In certain embodiments, the Q group of formulae I-a and IV is substituted with a group suitable for Click chemistry. Click reactions tend to involve high-energy (“spring-loaded”) reagents with well-defined reaction coordinates, that give rise to selective bond-forming events of wide scope. Examples include nucleophilic trapping of strained-ring electrophiles (epoxide, aziridines, aziridinium ions, episulfonium ions), certain carbonyl reactivity (e.g., the reaction between aldehydes and hydrazines or hydroxylamines), and several cycloaddition reactions. The azide-alkyne 1,3-dipolar cycloaddition is one such reaction. Click chemistry is known in the art and one of ordinary skill in the art would recognize that certain substituents on Q of the present invention are suitable for Click chemistry.

Compounds of formulae I-a and IV having substituents on Q that are suitable for Click chemistry are useful for conjugating said compounds to biological systems or macromolecules such as proteins, viruses, and cells, to name but a few. The Click reaction is known to proceed quickly and selectively under physiological conditions. In contrast, most conjugation reactions are carried out using the primary amine functionality on proteins (e.g. lysine or protein end-group). Because most proteins contain a multitude of lysines and arginines, such conjugation occurs uncontrollably at multiple sites on the protein. This is particularly problematic when lysines or arginines are located around the active site of an enzyme or other biomolecule. Thus, another embodiment of the present invention provides a method of conjugating a substituent on the Q group of a compound of formulae I-a and IV to a macromolecule via Click chemistry. Yet another embodiment of the present invention provides a macromolecule conjugated to a compound of formulae I-a and IV via a substituent on the Q group.

Before or after conjugation of compounds of formula IV to a biomolecule, drug, cell, substrate, or the like via a substituent on Q, the end-group functionalities, corresponding to free amine or salt thereof, group of formula IV, can be used to attach targeting groups for cell-specific delivery including, but not limited to, detectable moieties, such as fluorescent dyes, covalent attachment to surfaces, and incorporation into hydrogels.

According to one embodiment, the Q group of formulae I-a and IV is substituted with an azide-containing group. According to another embodiment, the Q group of formulae I-a and IV is substituted with an alkyne-containing group. In certain embodiments, the substituent on Q comprises a terminal alkyne moiety. In other embodiments, the Q group of formulae I-a and IV is substituted with an alkyne moiety having an electron withdrawing group. Accordingly, in such embodiments, the substituent on Q is

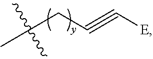

wherein E is an electron withdrawing group and y is 0-6. Such electron withdrawing groups are known to one of ordinary skill in the art. In certain embodiments, E is an ester. In other embodiments, the substituent on Q is

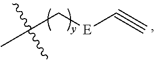

wherein E is an electron withdrawing group, such as a —C(O)O— group and y is 0-6.

Exemplary compounds of formula I-a include:

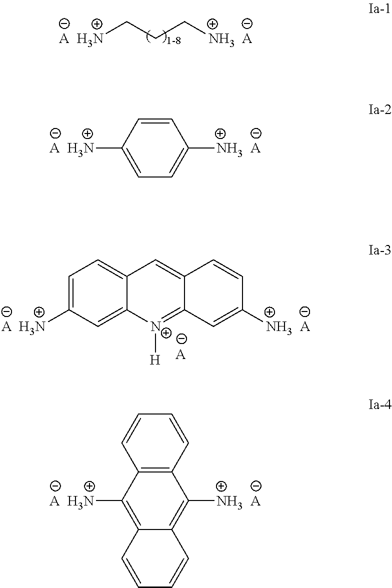

It will be appreciated by one of ordinary in the art that a compound of formula IV may be further derivatized by treatment of that compound with a suitable terminating agent. Thus, another embodiment of the present invention provides a compound of formula IV-a:

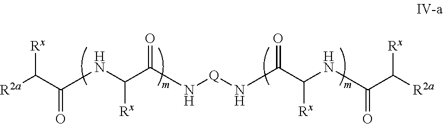

wherein:
m is 1-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} is a natural or unnatural amino acid side-chain group;
R^{2a} is a mono-protected amine, a di-protected amine, —NHR³, —N(R³)₂, —NHC(O)R³, —NR³C(O)R³, —NHC(O)NHR³, —NHC(O)N(R³)₂, —NR³C(O)NHR³, —NR³C(O)N(R³)₂, —NHC(O)OR³, —NR³C(O)OR³, —NHSO₂R³, or —NR³SO₂R³; and
   each R³ is independently an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or:
   two R³ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

Each of the embodiments relating to the Q, m, R^{x}, and R^{2a} groups of formulae II, II-a, I-a, and IV apply to the Q, m, R^{x}, and R^{2a} groups of formula IV-a both singly and in combination.

Compounds of formula I-a are also useful for preparing poly(amino acid) multi-block copolymers of formula IV-b:

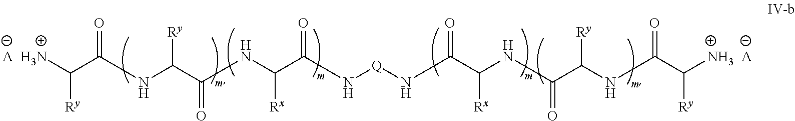

wherein each A is a suitable acid anion and each Q, m, m′, R^{y}, and R^{x} are as defined above and in classes and subclasses described herein.

Accordingly, another aspect of the present invention provides a compound of formula IV-b:

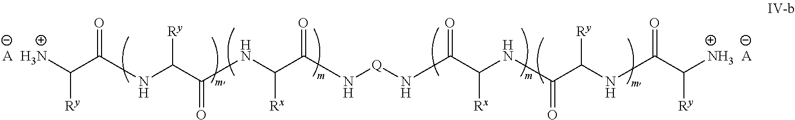

wherein:
m is 1-1000;
m′ is 0-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} and R^{y} are each independently a natural or unnatural amino acid side-chain group, wherein R^{x} and R^{y} are different from each other; and
   each A is a suitable acid anion.

Each of the embodiments relating to the Q, m, m′, R^{x} and R^{y} groups of formulae II, I-a, and IV apply to the Q, m, m′, R^{x} and R^{y} groups of formula IV-b both singly and in combination.

It will be appreciated by one of ordinary in the art that a compound of formula IV-b may be further derivatized by treatment of that compound with a suitable terminating agent. Thus, another embodiment of the present invention provides a compound of formula IV-c:

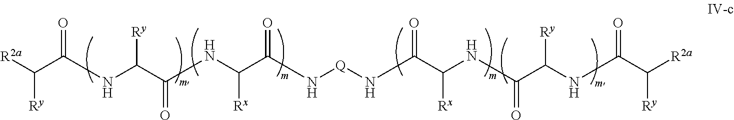

wherein:
m and m′ are each independently 1-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} and R^{y} are each independently a natural or unnatural amino acid side-chain group, wherein R^{x} and R^{y} are different from each other;
R^{2a} is a mono-protected amine, a di-protected amine, —NHR³, —N(R³)₂, —NHC(O)R³, —NR³C(O)R³, —NHC(O)NHR³, —NHC(O)N(R³)₂, —NR³C(O)NHR³, —NR³C(O)N(R³)₂, —NHC(O)OR³, —NR³C(O)OR³, —NHSO₂R³, or —NR³SO₂R³; and
each R³ is independently an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or:
   two R³ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

Each of the embodiments relating to the Q, m, m′, R^{x}, R^{y}, and R^{2a} groups of formulae II, II-a, I-a, and IV apply to the Q, m, m′, R^{x}, R^{Y}, and R^{2a} groups of formula IV-c both singly and in combination.

As described above, the block poly(amino acid) compounds of the present invention may be PEGylated. Accordingly, another embodiment of the present invention relates to a compound of formula V:

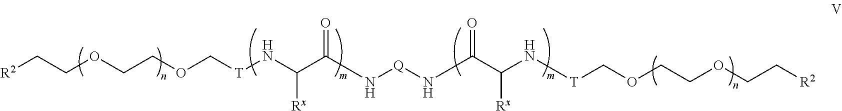

wherein:
each n is independently 10-2500;
each m is independently 1-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} is a natural or unnatural amino acid side-chain group;
each T is independently a valence bond or a bivalent, saturated or unsaturated, straight or branched C₁₋₁₂ alkylene chain, wherein 0-6 methylene units of Q are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—,
   wherein:
      each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each R is independently hydrogen or an optionally substituted aliphatic group; and
each R² is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³;
   q and r are each independently 0-4;
   each R³ is independently an optionally substituted group selected from aliphatic, a 5-8-membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
      two R³ on the same nitrogen atom are taken together with said nitrogen atom to form an optionally substituted 4-7-membered saturated, partially unsaturated, or aryl ring having 1-4 heteroatoms independently selected from nitrogen, oxygen, or sulfur; and
   R⁴ is hydrogen, halogen, N₃, CN, a mono-protected amine, a di-protected amine, a protected aldehyde, a protected hydroxyl, a protected carboxylic acid, a protected thiol, or an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety.

Each of the embodiments relating to the Q, m, and R^{x} groups of formulae II, I-a, and IV apply to the Q, m, and R^{x} groups of formula V both singly and in combination. In addition, each of the embodiments relating to the R² and T groups of formula III apply to the R² and T groups of formula V.

Yet another embodiment relates to a compound of formula V-a:

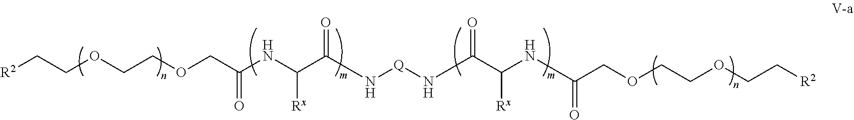

wherein:
each n is independently 10-2500;
each m is independently 1-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} is a natural or unnatural amino acid side-chain group; and
each R² is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³;
   q and r are each independently 0-4;
   each R³ is independently an optionally substituted group selected from aliphatic, a 5-8-membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
      two R³ on the same nitrogen atom are taken together with said nitrogen atom to form an optionally substituted 4-7-membered saturated, partially unsaturated, or aryl ring having 1-4 heteroatoms independently selected from nitrogen, oxygen, or sulfur; and
   R⁴ is hydrogen, halogen, N₃, CN, a mono-protected amine, a di-protected amine, a protected aldehyde, a protected hydroxyl, a protected carboxylic acid, a protected thiol, or an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety.

Each of the embodiments relating to the Q, m, and R^{x} groups of formulae II, I-a, and IV apply to the Q, m, and R^{x} groups of formula V-a both singly and in combination. In addition, each of the embodiments relating to the R² group of formula III apply to the R² group of formula V-a.

In certain embodiments, the present invention provides a compound of formula VI:

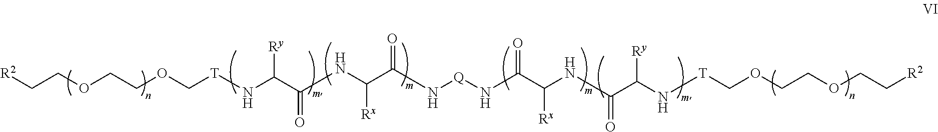

wherein:
each n is independently 10-2500;
m is 1-1000;
m′ is 0-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} and R^{y} are each independently a natural or unnatural amino acid side-chain group, wherein R^{x} and R^{y} are different from each other;
each T is independently a valence bond or a bivalent, saturated or unsaturated, straight or branched C₁₋₁₂ alkylene chain, wherein 0-6 methylene units of Q are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—,
   wherein:
      each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each R² is halogen, N₃, CN, a mono-protected amine, a di-protected amine, a protected hydroxyl, a protected aldehyde, a protected thiol, —N(R³)₂, —SR³, —O(CH₂CH₂O)_{q}(CH₂)ᵣR⁴, —OC(O)R³, or —OS(O)₂R³;
   q and r are each independently 0-4;
   each R³ is independently an optionally substituted group selected from aliphatic, a 5-8-membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
      two R³ on the same nitrogen atom are taken together with said nitrogen atom to form an optionally substituted 4-7-membered saturated, partially unsaturated, or aryl ring having 1-4 heteroatoms independently selected from nitrogen, oxygen, or sulfur; and
   R⁴ is hydrogen, halogen, N₃, CN, a mono-protected amine, a di-protected amine, a protected aldehyde, a protected hydroxyl, a protected carboxylic acid, a protected thiol, or an optionally substituted group selected from aliphatic, a 5-8-membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety.

Each of the embodiments relating to the Q, m, m′, R^{x} and R^{y} groups of formulae II, I-a, and IV apply to the Q, m, m′, R^{x} and R^{y} groups of formula VI both singly and in combination. In addition, each of the embodiments relating to the R² and T groups of formula III apply to the R² and T groups of formula VI.

Another embodiment of the present invention relates to a compound of formula VI-a:

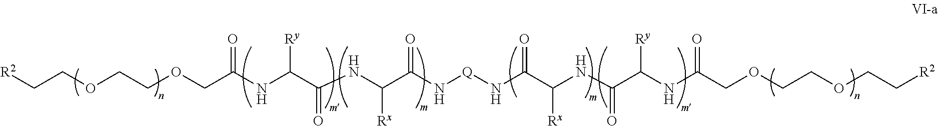

wherein:
each n is independently 10-2500;
m is 1-1000;
m′ is 0-1000;
Q is an optionally substituted bivalent group selected from a C₁₋₆ aliphatic group, a 3-7 membered saturated, partially unsaturated, or aryl monocycle ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} and R^{y} are each independently a natural or unnatural amino acid side-chain group, wherein R^{x} and R^{y} are different from each other; and
each R² is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³;
   q and r are each independently 0-4;
   each R³ is independently an optionally substituted group selected from aliphatic, a 5-8-membered saturated, partially unsaturated, or aryl ring having 0-4 heteroatoms independently selected from nitrogen, oxygen, or sulfur, an 8-10-membered saturated, partially unsaturated, or aryl bicycle ring having 0-5 heteroatoms independently selected from nitrogen, oxygen, or sulfur, or a detectable moiety, or:
      two R³ on the same nitrogen atom are taken together with said nitrogen atom to form an optionally substituted 4-7-membered saturated, partially unsaturated, or aryl ring having 1-4 heteroatoms independently selected from nitrogen, oxygen, or sulfur; and
   R⁴ is hydrogen, halogen, N₃, CN, a mono-protected amine, a di-protected amine, a protected aldehyde, a protected hydroxyl, a protected carboxylic acid, a protected thiol, or an optionally substituted group selected from aliphatic, a 5-8-membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety.

Each of the embodiments relating to the Q, m, m′, R^{x} and R^{y} groups of formulae II, I-a, and IV apply to the Q, m, m′, R^{x} and R^{y} groups of formula VI-a both singly and in combination. In addition, each of the embodiments relating to the R² group of formula III apply to the R² group of formula VI-a.

In other embodiments, it is contemplated that the R¹ group of formula I includes amine-terminal dendritic groups. Such dendritic R¹ groups are particularly useful for preparing star-block poly(amino acid) copolymers using the methods of the present invention. In particular, star-block poly(amino acid) copolymers can be synthesized by sequential addition of NCAs to multi-functional amine salt initiators. The number of amine salts on the initiating species dictates the number of polymer arms. Using amine-terminal, dendritic cores offer an effective method to make highly branched star polymers. Generation I and II polypropyleneimine (DAB-AM) dendrimers are used to make 4 and 8 arm star polymers, respectively. Generations 3, 4, and 5 DAB-AM dendrimers are used to synthesize 16, 32, and 64 arm star polymers, respectively. Alternatively, poly(amidoamine) (PAMAM) dendrimers could be used in this capacity. Examples of such multi-functional initiators include:

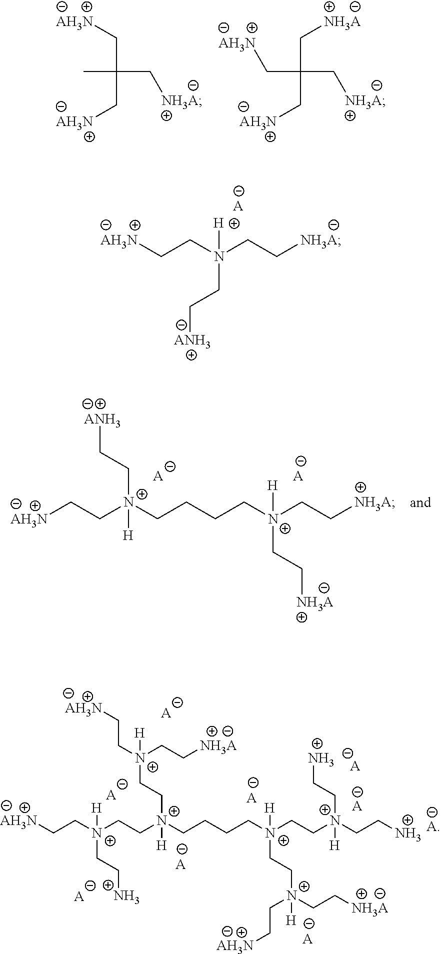

Such multi-functional initiators are useful for preparing star-block poly(amino acid) copolymers using the methods of the present invention.

### 4. General Methods of Providing the Present Compounds

The compounds of this invention may be prepared or isolated in general by synthetic and/or pseudo-synthetic methods known to those skilled in the art for analogous compounds and as illustrated by the general schemes that follow.

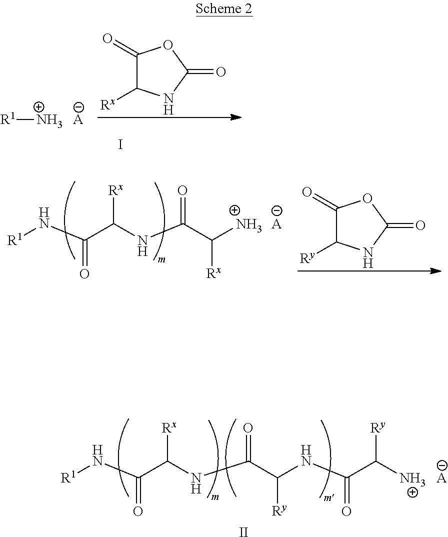

Scheme 2 above depicts a general method for preparing compounds of formula II of the present invention. In particular, Scheme 2 depicts the sequential polymerization of amino acid NCA's for preparing compounds of the present invention having multiple poly(amino acid) blocks.

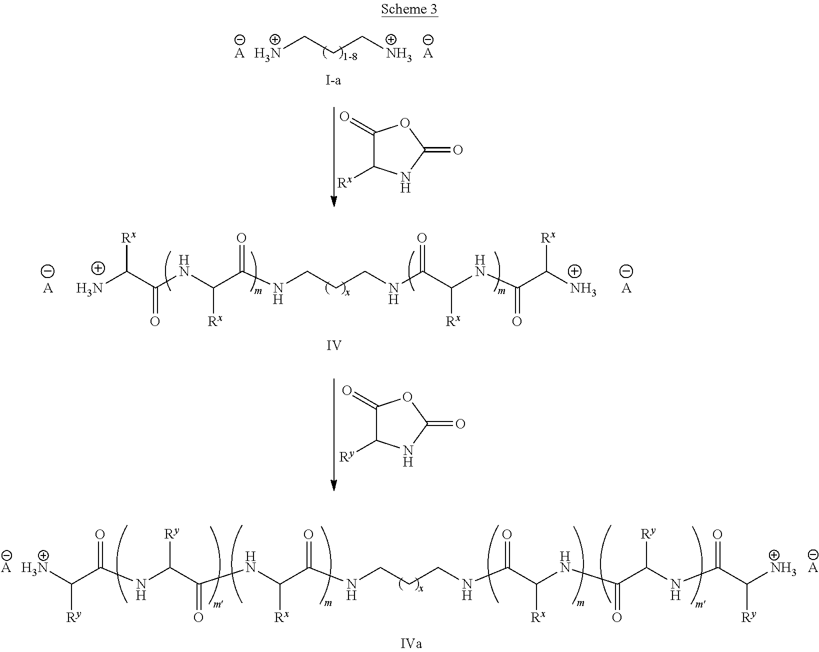

Scheme 3 above depicts a general method for preparing compounds of formula IV and IVa from a compound of formula I-a.

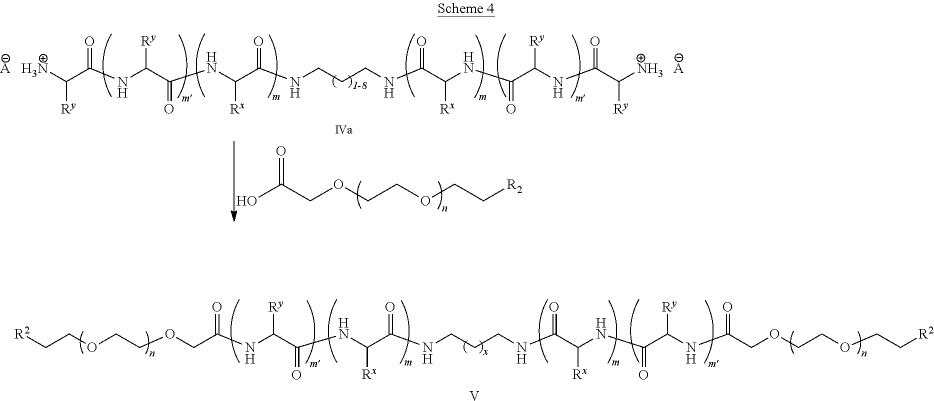

Scheme 4 above depicts a general method for preparing compounds of formula V from a compound of formula IVa by coupling a PEG-carboxylate onto the amine terminal ends of formula IVa. One of ordinary skill in the art would recognize that the coupling step, as depicted above, is performed using a variety of coupling methods. Such methods include, but are not limited to, activated ester formation, acyl halide coupling, and the like.

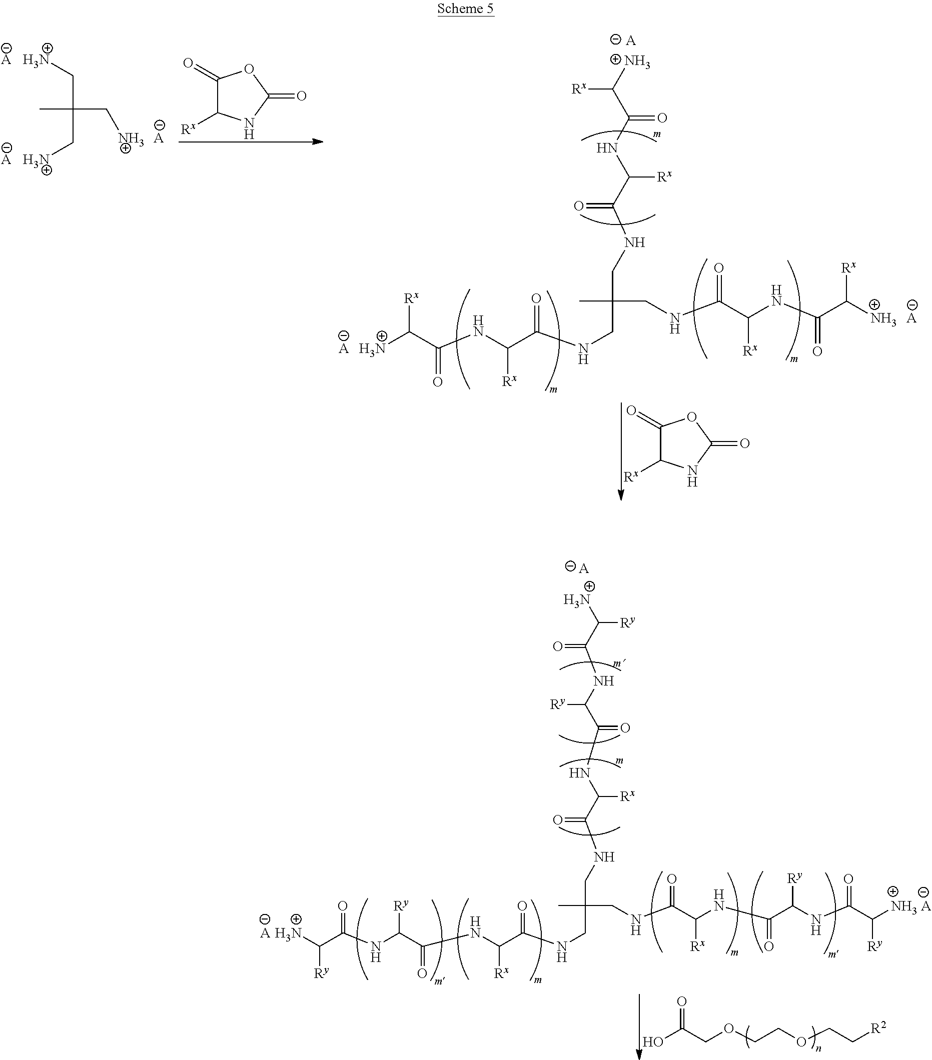

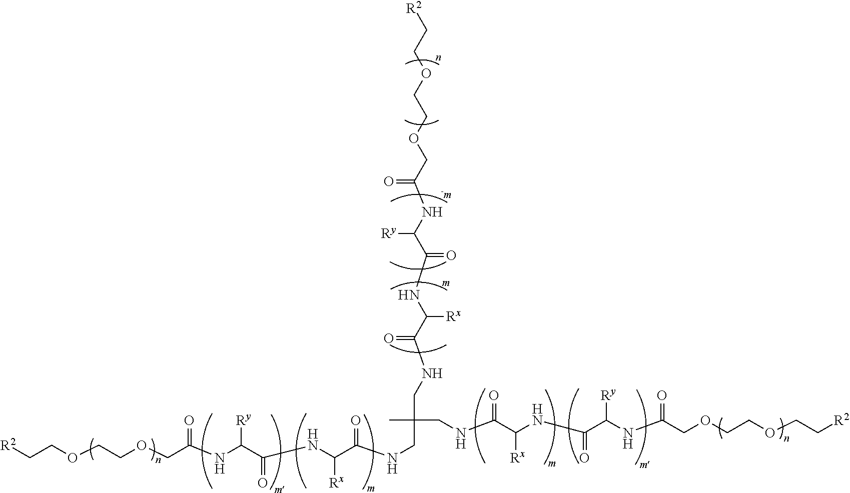

Scheme 5 above shows a general method for preparing compounds of the present invention wherein R¹ is an amine-terminal dendritic compound.

Although certain exemplary embodiments are depicted and described above and herein, it will be appreciated that compounds of the invention can be prepared according to the methods described generally above using appropriate starting materials by methods generally available to one of ordinary skill in the art. Additional embodiments are exemplified in more detail herein.

### 5. Uses, Methods, and Compositions

As discussed above, the present invention provides homopolymers or block copolymers, intermediates thereto, and methods of preparing the same. Such homopolymers and block copolymers are useful for a variety of purposes in the pharmaceutical and biomedical fields. Such uses include using the homopolymers and block copolymers of the present invention, and in certain embodiments, the PEG-poly (amino acid) block copolymers prepared by the methods of the present invention in the process of conjugating other molecules.

Amphiphilic multi-block copolymers, as described herein, can self-assemble in aqueous solution to form nano- and micron-sized structures, with applications from drug encapsulation to artificial viruses and cells. In water, these amphiphilic copolymers assemble by multi-molecular micellization when present in solution above the critical micelle concentration (CMC). Without wishing to be bound by any particular theory, it is believed that the hydrophobic poly(amino acid) portion or “block” of the copolymer collapses to form the micellar core, while the hydrophilic PEG block forms a peripheral corona and imparts water solubility. Additionally, poly(amino acid) blocks capable of chemical crosslinking (e.g. aspartic and glutamic acid, cysteine, or serine) may also be incorporated into the amphiphilic copolymer to further enhance the stability of micellar assemblies. These core-shell polymer micelles can be tuned to encapsulate a variety of therapeutic molecules, including small molecule drugs, polypeptides, and polynucleotides.

One of ordinary skill in the art would recognize that the compounds prepared by the methods of the present invention are useful for either encapsulating or conjugating small molecule drugs. In certain embodiments, the present compounds are used to PEGylate such drugs. Small molecule drugs suitable for PEGylation, conjugation, or encapsulation with the compounds prepared by the methods of the present invention include, without limitation, chemotherapeutic agents or other anti-proliferative agents including alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), angiogenesis inhibitors (Avastin) and hormones (Tamoxifen, Leuprolide, Flutamide, and Megestrol), Gleevec, dexamethasone, and cyclophosphamide. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Other examples of small molecule drugs that may be PEGylated, conjugated, or encapsulated with the compounds prepared by the methods of this invention include treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

In order that the invention described herein may be more fully understood, the following examples are set forth. It will be understood that these examples are for illustrative purposes only and are not to be construed as limiting this invention in any manner.

### EXAMPLES

### Example 1

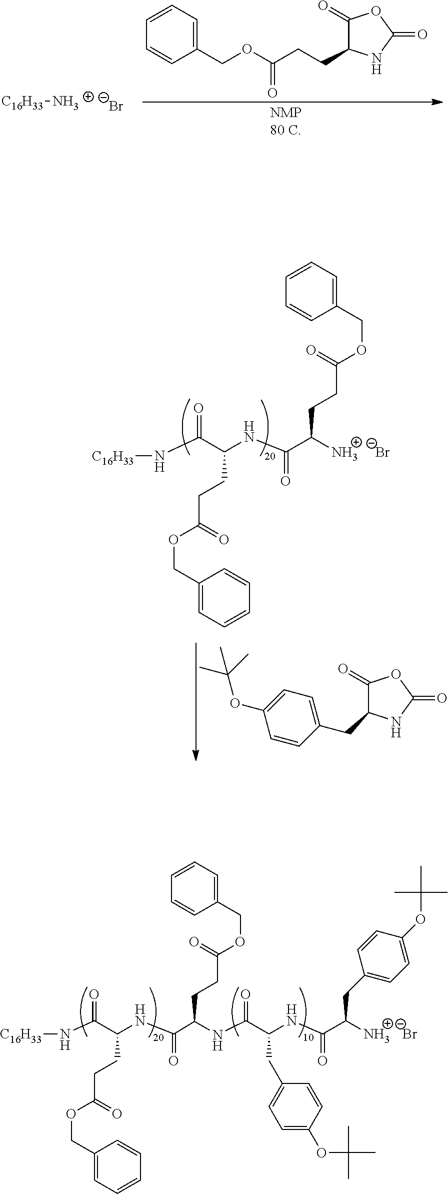

To a 100 mL flask equipped with glass stir bar, valve and septum was added hexadecylamine-hydrobromide salt (49 mg, 0.15 mmol) and benzyl glutamate NCA (0.8 g, 3.0 mmol). The contents were dried under vacuum for 1 h then backfilled with Argon. Anhydrous NMP (10 mL) was added via syringe then the flask sealed and stirred at 80° C. Aliquots were removed from the reaction vessel using Schienk technique every 24 hours. After 72 hours, t-butyl tyrosine NCA (0.4 g, 1.5 mmol) in anhydrous NMP (2 mL) was added via syringe. After an additional 72 hours, the solution was cooled and precipitated into cold ether giving yellow solid (0.6 g). Analysis of the aliquots by size exclusion chromatography giving peak molecular weights relative to PEG standards: Mp=780, PDI=1.06 (24 hours); Mp=1000, PDI=1.04 (72 hours); Mp=1240, PDI=1.14 (144 hours). ¹H NMR (δ, DMSO-d₆, 400 MHz) 9.19, 8.07, 7.33, 7.00, 6.62, 5.05, 4.27, 3.28, 2.69, 2.60, 2.37, 1.93, 1.21.

### Example 2

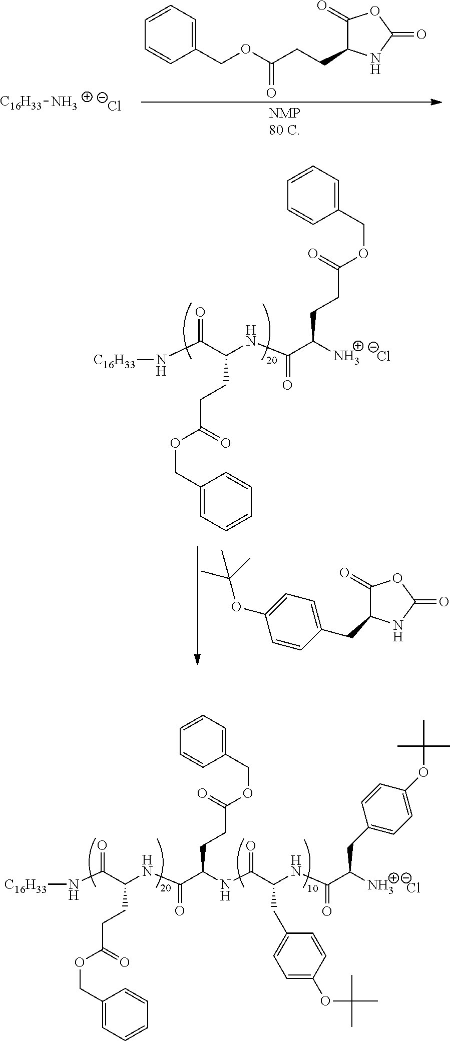

To a 100 mL flask equipped with glass stir bar, valve and septum was added hexadecylamine-hydrochloride salt (42 mg, 0.15 mmol) and benzyl glutamate NCA (0.8 g, 3.0 mmol). The contents were dried under vacuum for 1 h then backfilled with Argon. Anhydrous NMP (10 mL) was added via syringe then the flask sealed and stirred at 80° C. Aliquots were removed from the reaction vessel using Schlenk technique every 24 hours. After 72 hours, t-butyl tyrosine NCA (0.4 g, 1.5 mmol) in anhydrous NMP (2 mL) was added via syringe. After an additional 72 hours, the solution was cooled and precipitated into cold ether giving yellow solid (0.5 g). Analysis of the aliquots by size exclusion chromatography giving peak molecular weights relative to PEG standards: Mp=840, PDI=1.05 (24 hours); Mp=1000, PDI=1.04 (72 hours); Mp=1110, PDI=1.14 (144 hours).

### Example 3

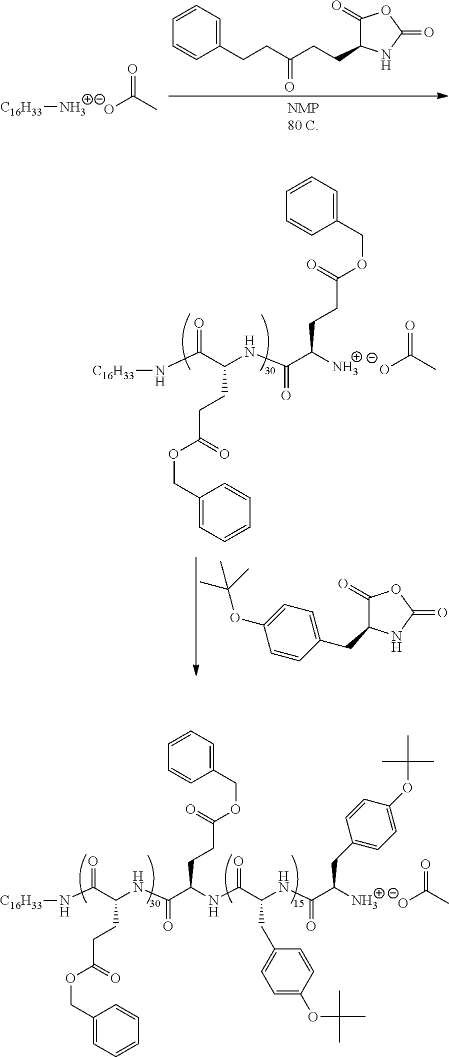

To a 100 mL flask equipped with glass stir bar, valve and septum was added hexadecylamine-acetic acid salt (39 mg, 0.13 mmol) and benzyl glutamate NCA (1.0 g, 3.8 mmol). The contents were dried under vacuum for 1 h then backfilled with Argon. Anhydrous NMP (10 mL) was added via syringe then the flask sealed and stirred at 80° C. Aliquots were removed from the reaction vessel using Schlenk technique every 24 hours. After 72 hours, t-butyl tyrosine NCA (0.5 g, 1.9 mmol) in anhydrous NMP (2 mL) was added via syringe. After an additional 72 hours, the solution was cooled and precipitated into cold ether giving yellow solid (0.9 g). Analysis of the aliquots by size exclusion chromatography giving peak molecular weights relative to PEG standards: Mp=2110, PDI=1.61 (72 hours); Mp=2560, PDI=1.95 (96 hours).

### Example 4

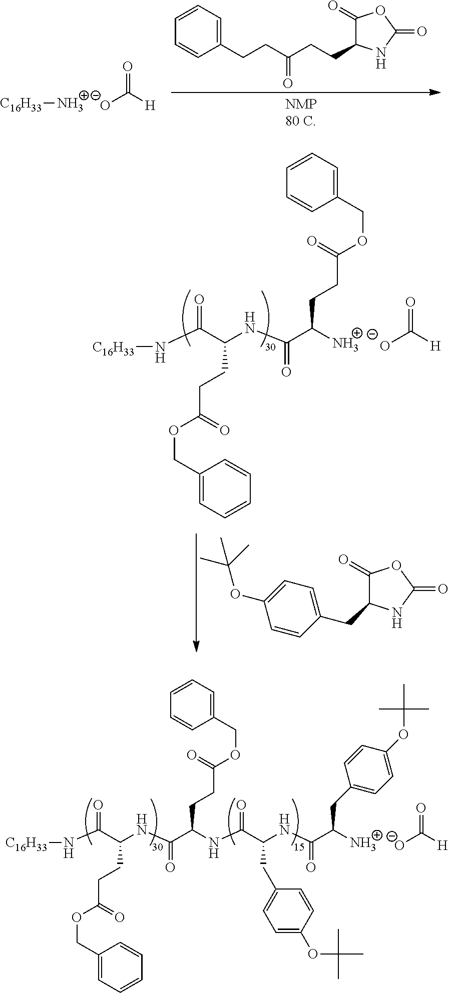

To a 100 mL flask equipped with glass stir bar, valve and septum was added hexadecylamine-formic acid salt (36 mg, 0.13 mmol) and benzyl glutamate NCA (1.0 g, 3.8 mmol). The contents were dried under vacuum for 1 h then backfilled with Argon. Anhydrous NMP (10 mL) was added via syringe then the flask sealed and stirred at 80° C. Aliquots were removed from the reaction vessel using Schlenk technique every 24 hours. After 72 hours, t-butyl tyrosine NCA (0.5 g, 1.9 mmol) in anhydrous NMP (2 mL) was added via syringe. After an additional 72 hours, the solution was cooled and precipitated into cold ether giving yellow solid (0.8 g). Analysis of the aliquots by size exclusion chromatography giving peak molecular weights relative to PEG standards: Mp=2550, PDI=1.68 (24 hours); Mp=4580, PDI=1.61 (96 hours).

### Example 5

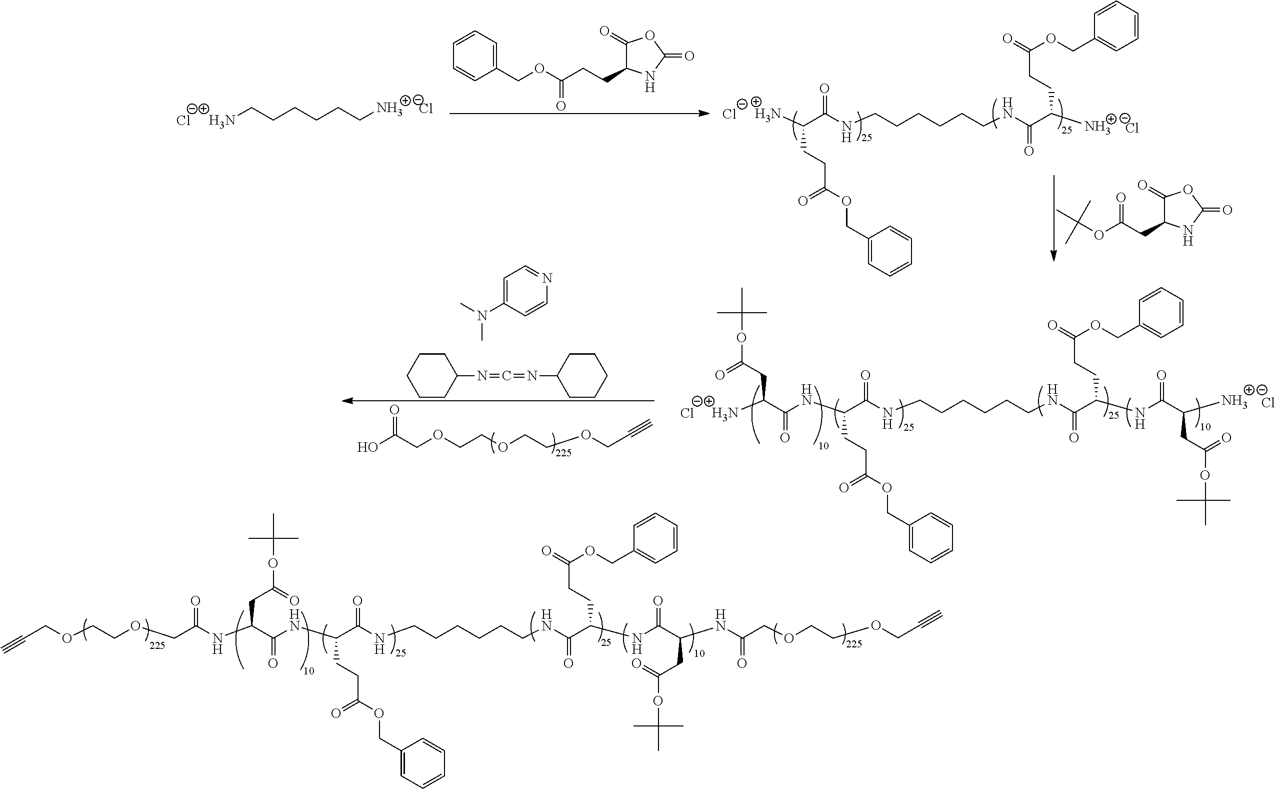

### Example 6

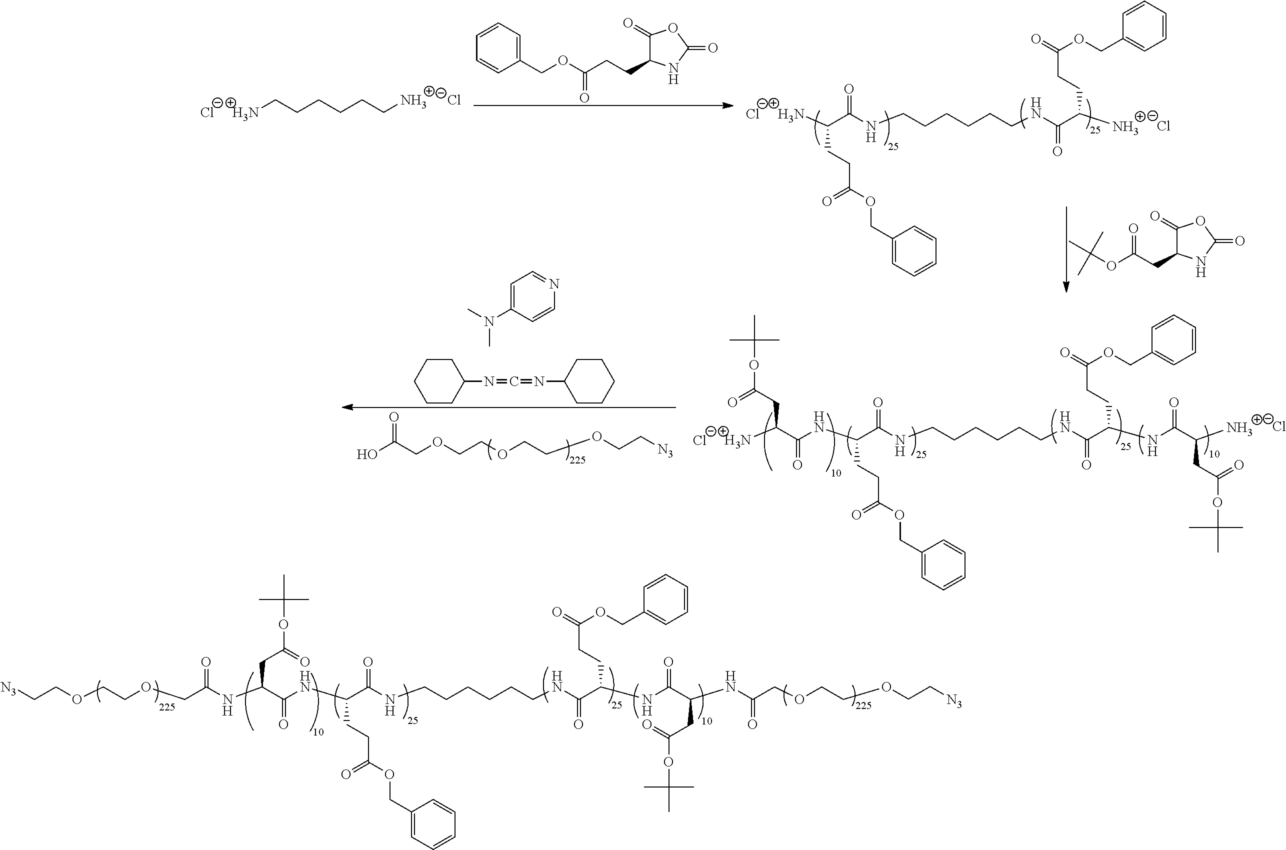

### Example 7

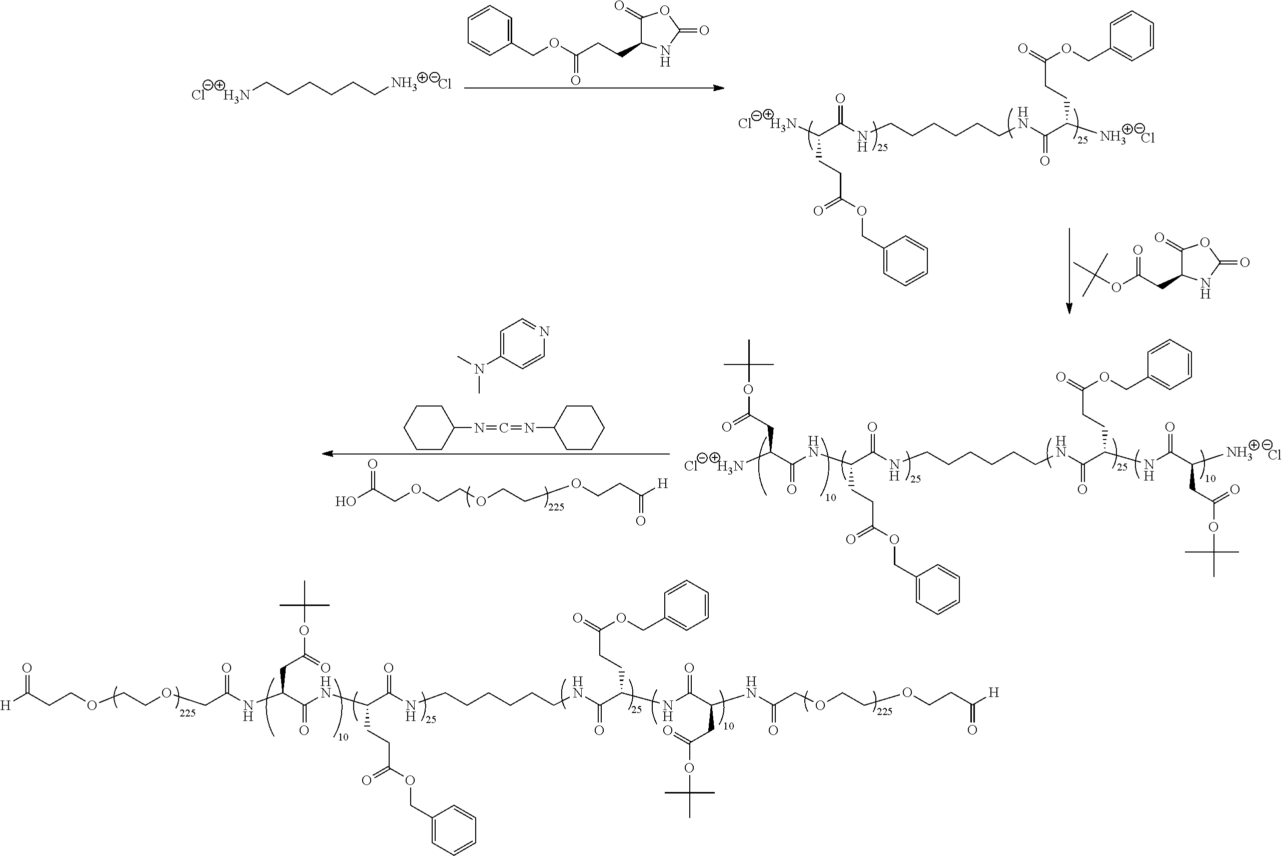

### Example 8

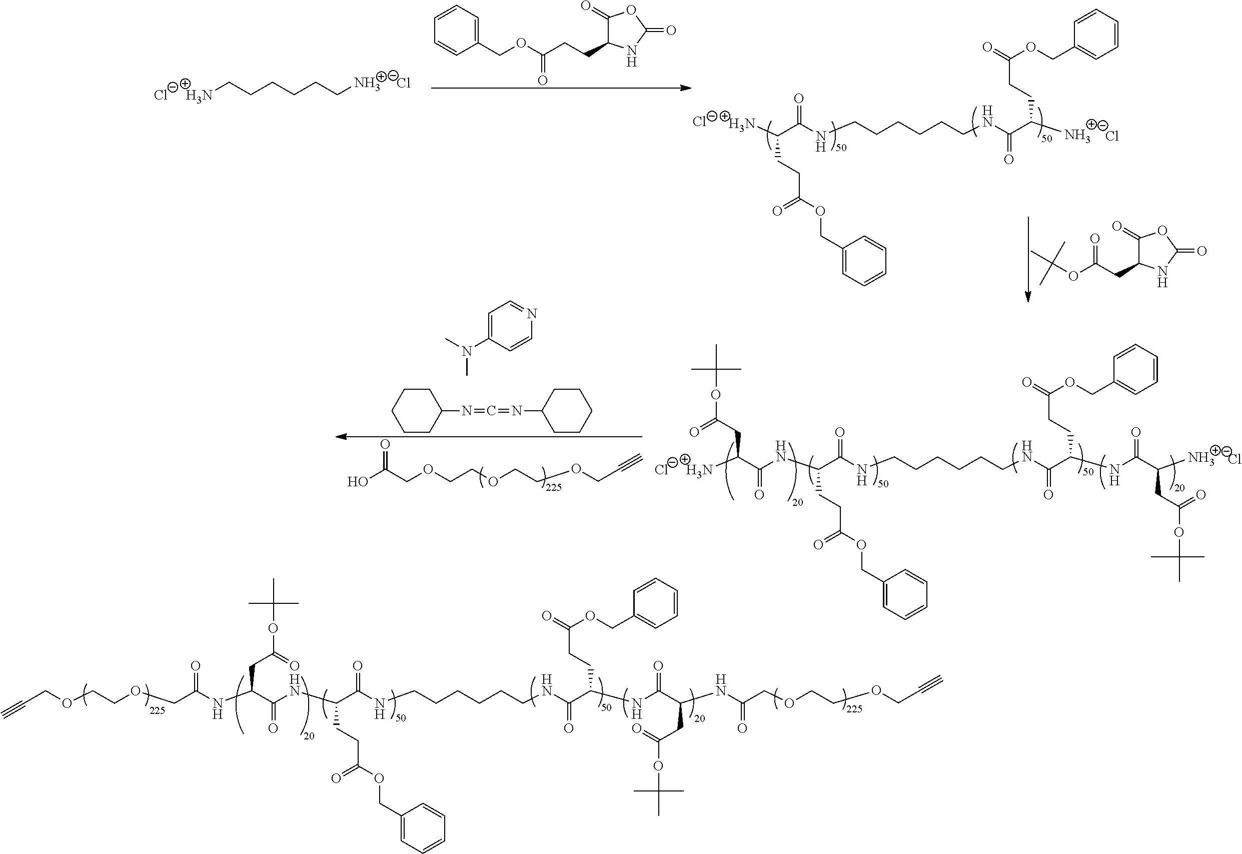

### Example 9

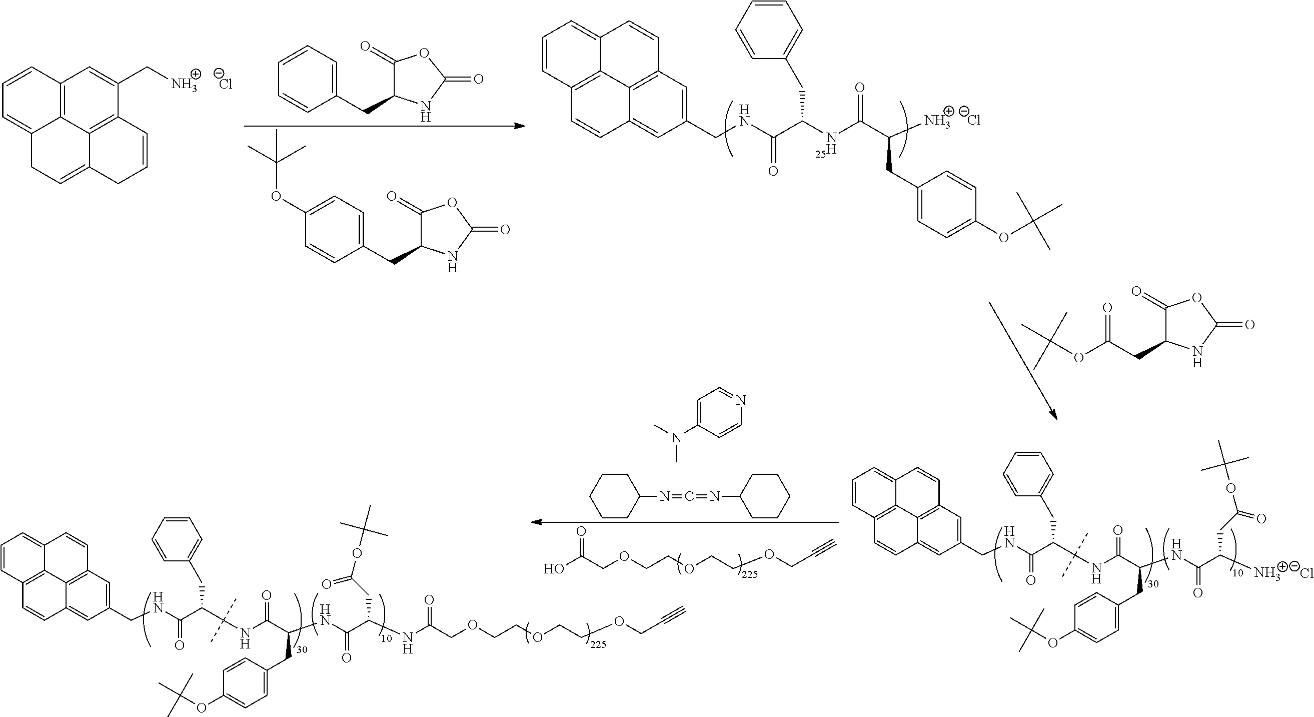

### Example 10

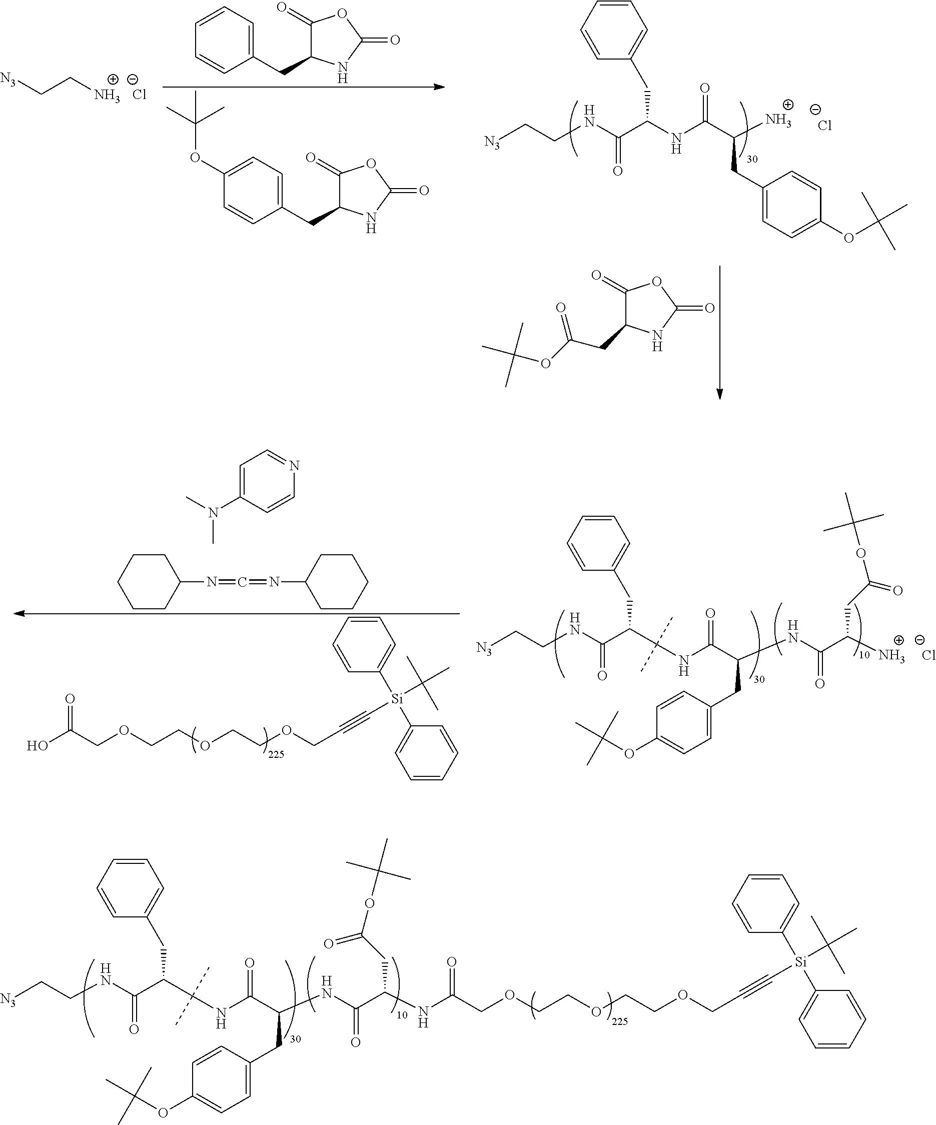

### Example 11

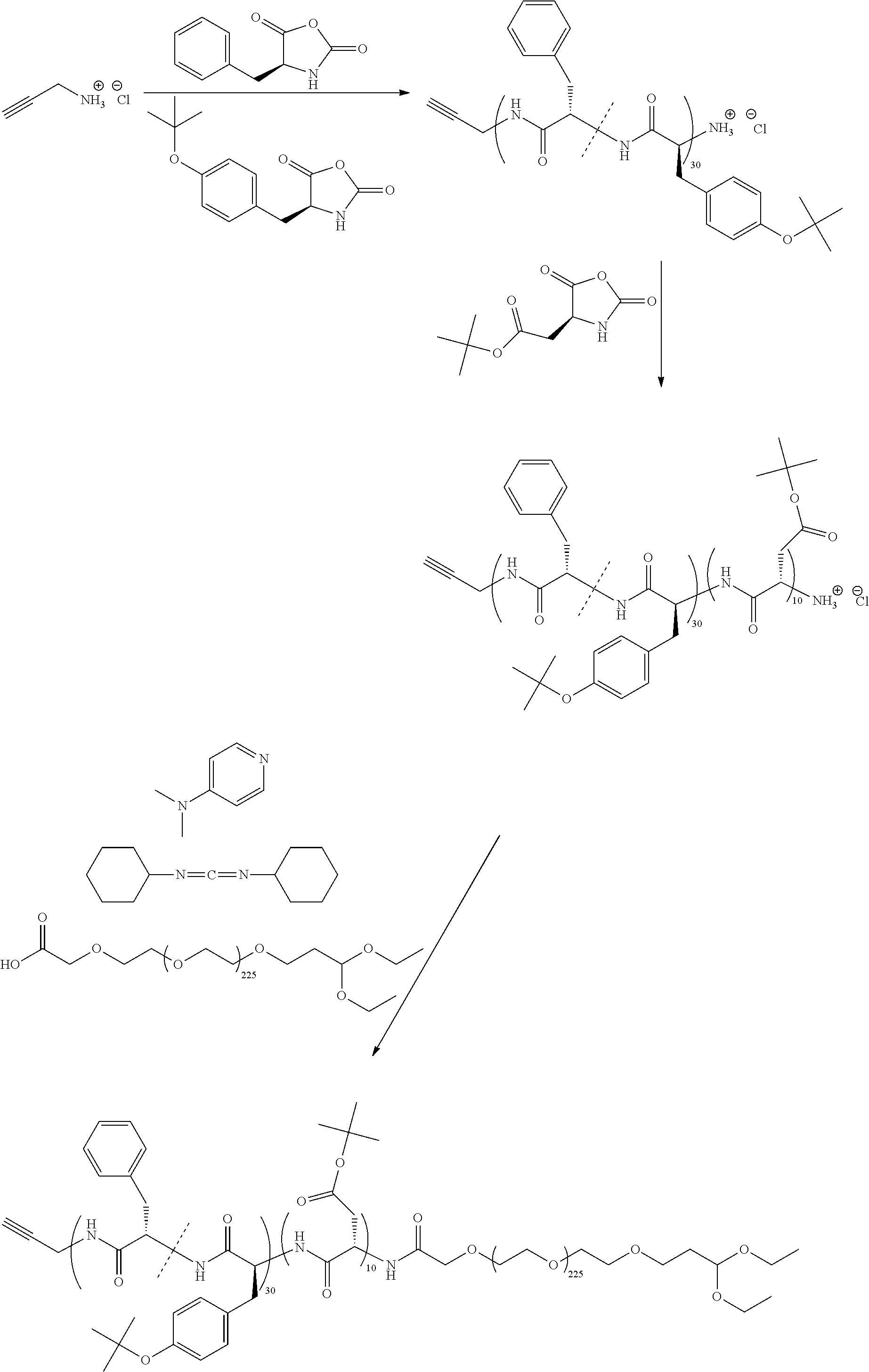

### Example 12

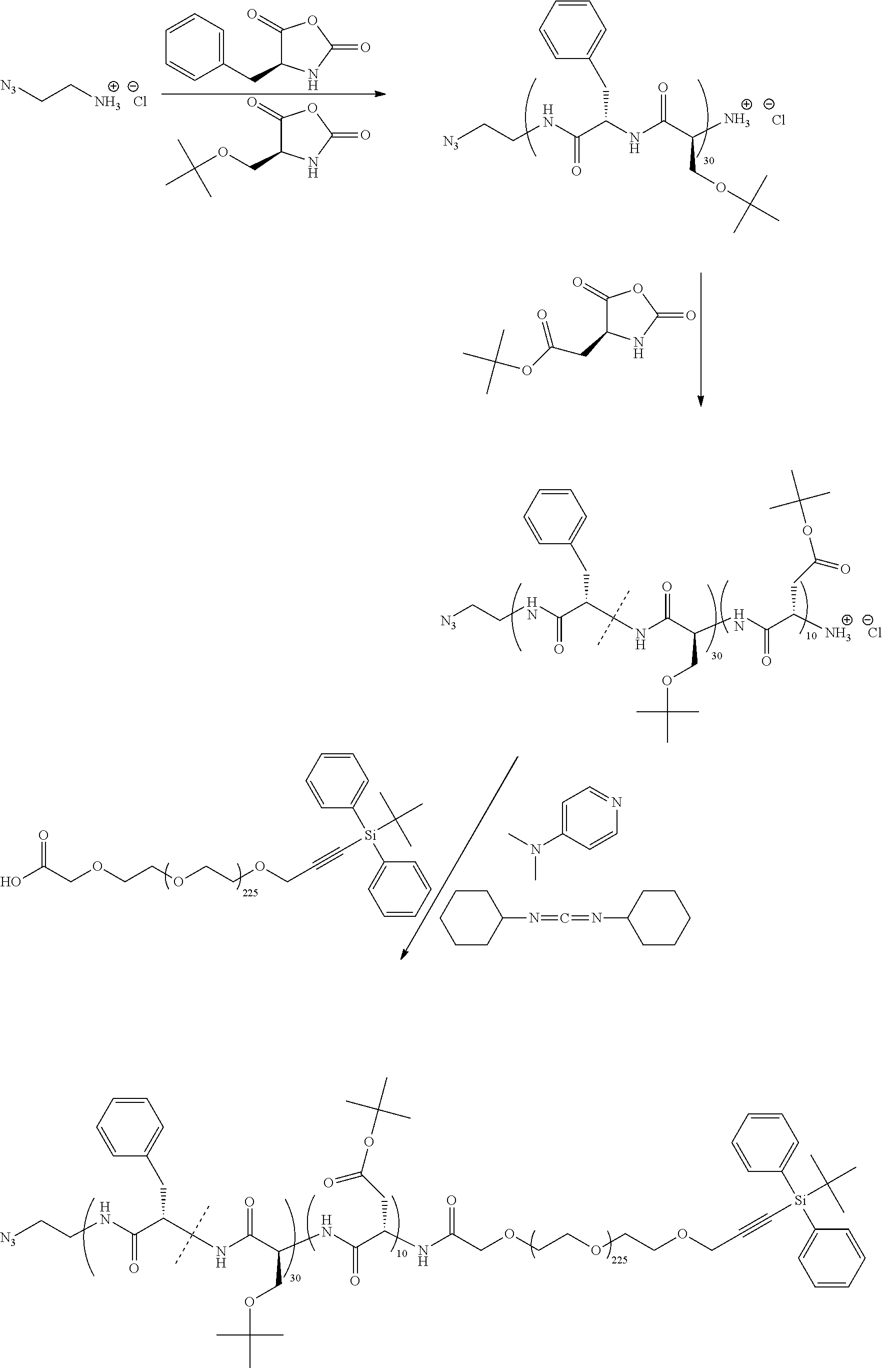

### Example 13

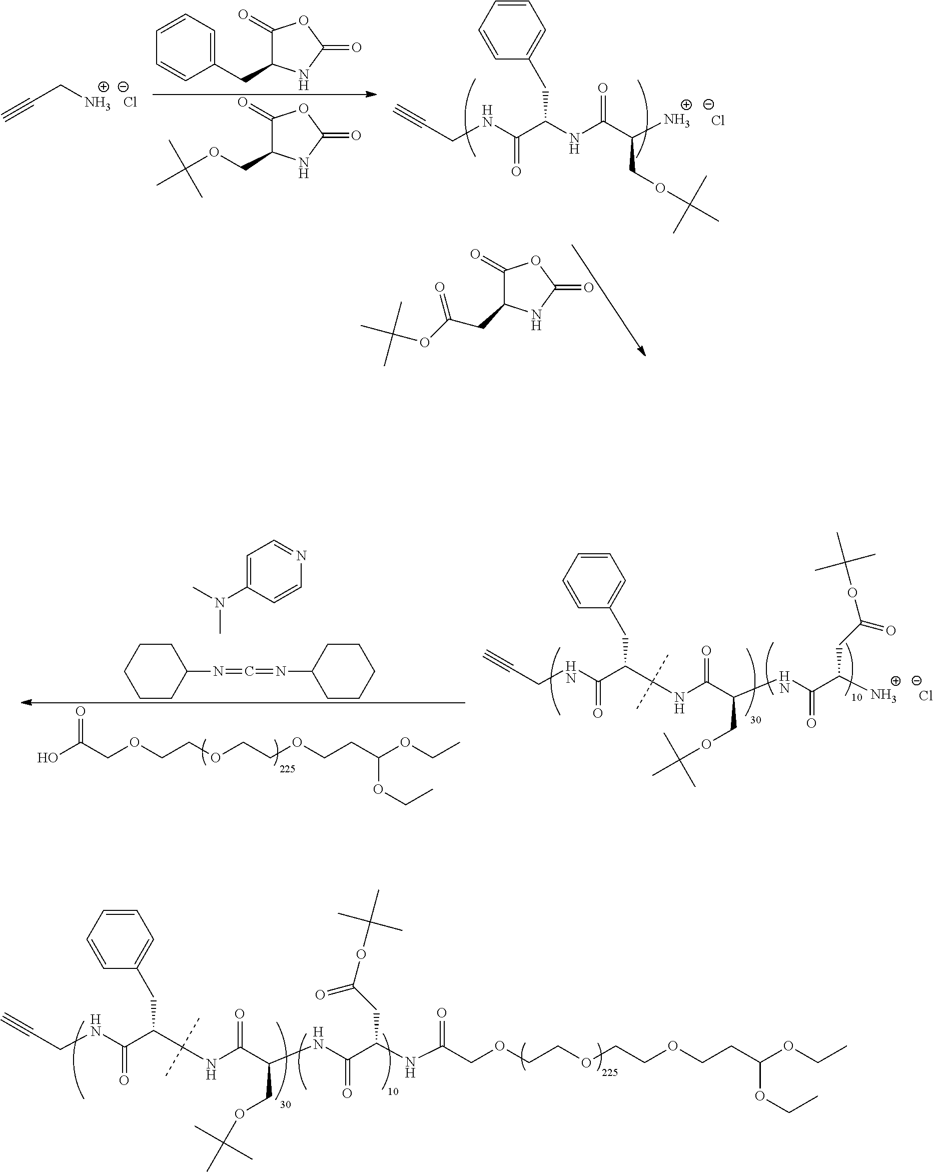

### Example 14

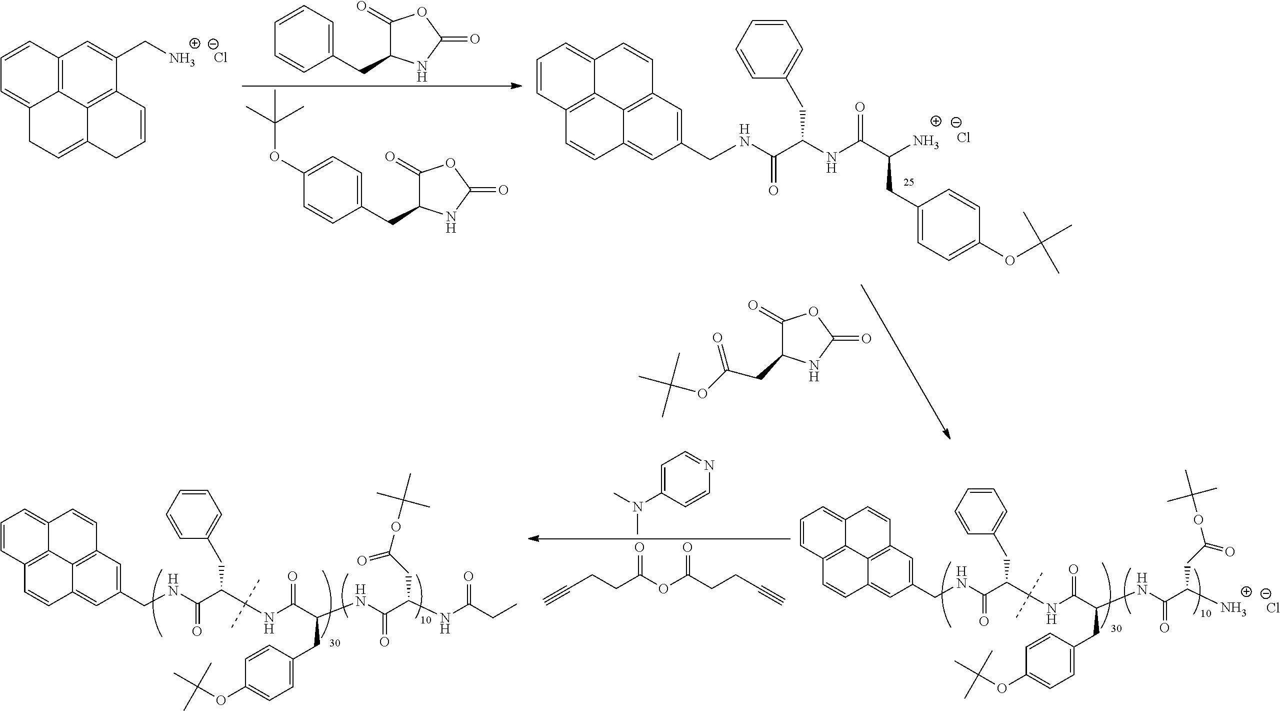

### Example 15

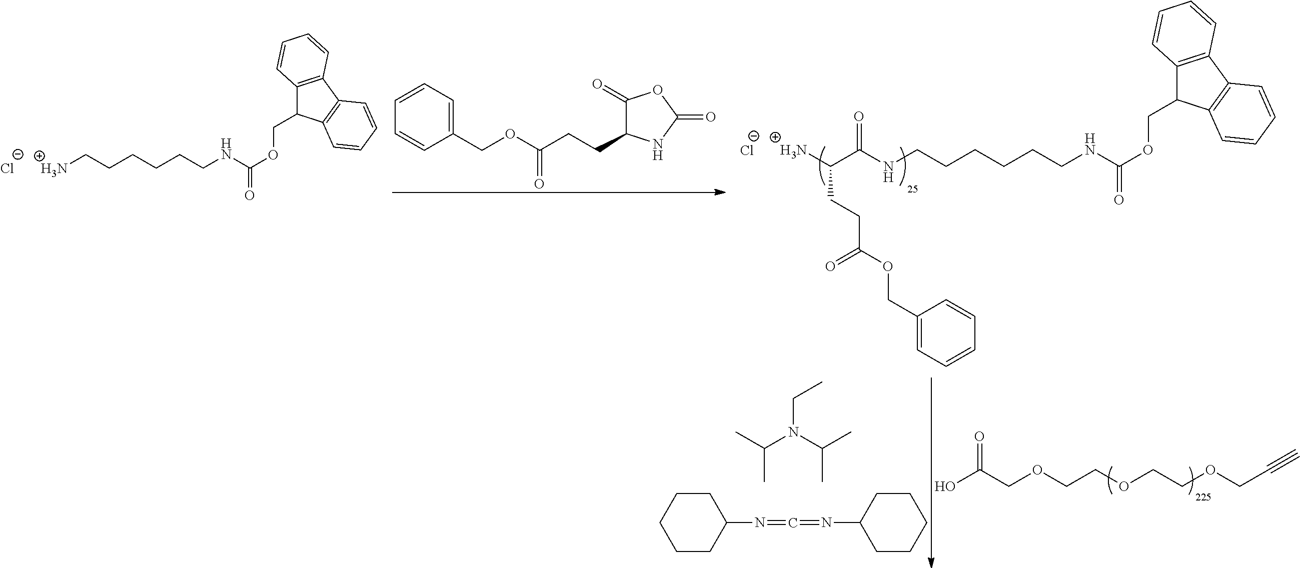

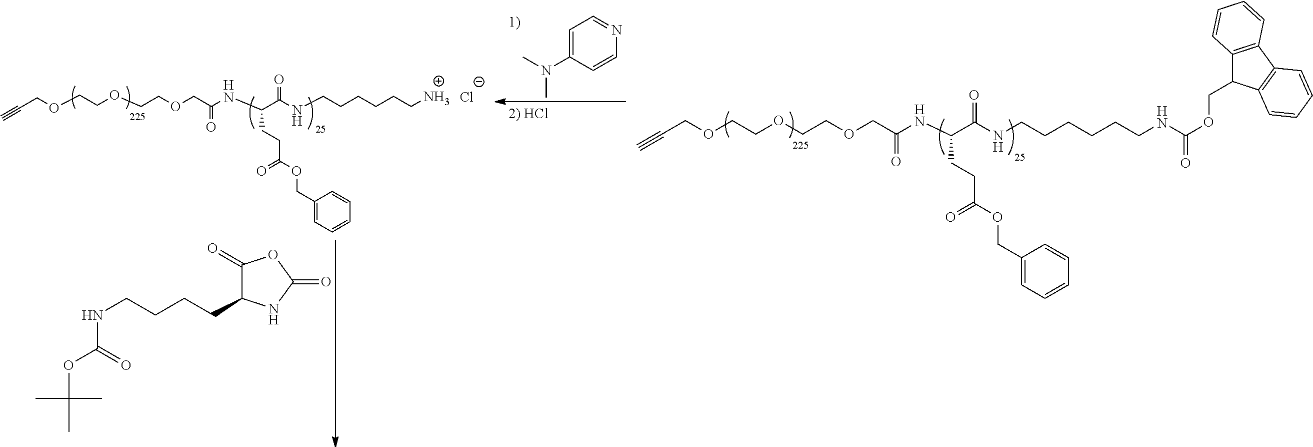

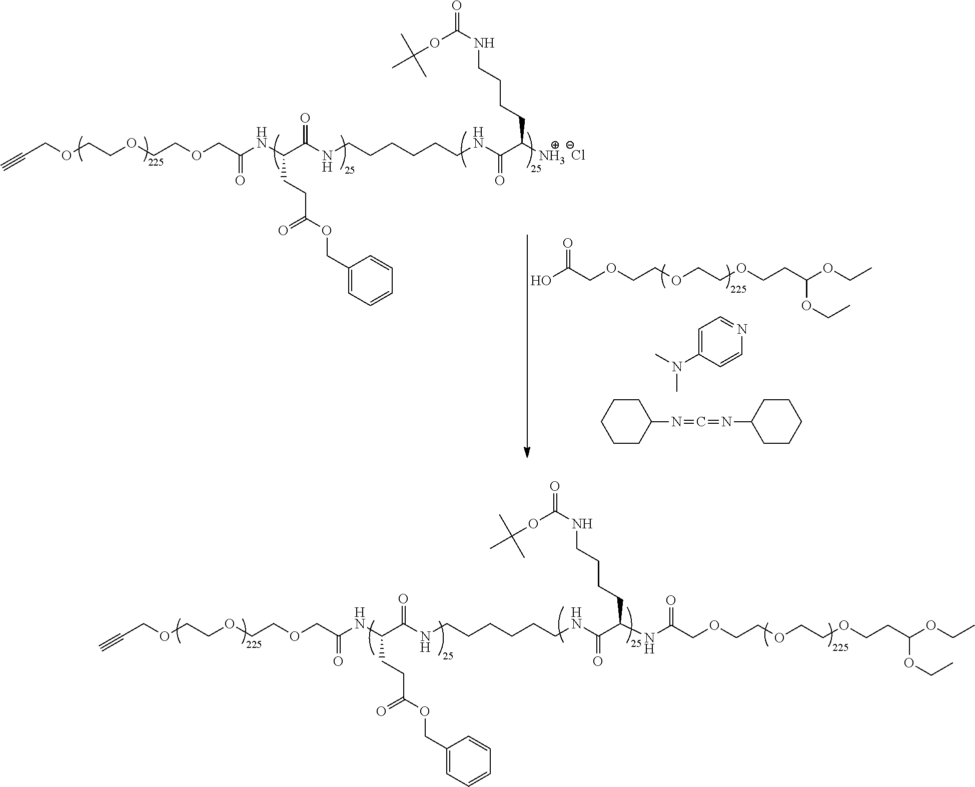

### Example 16

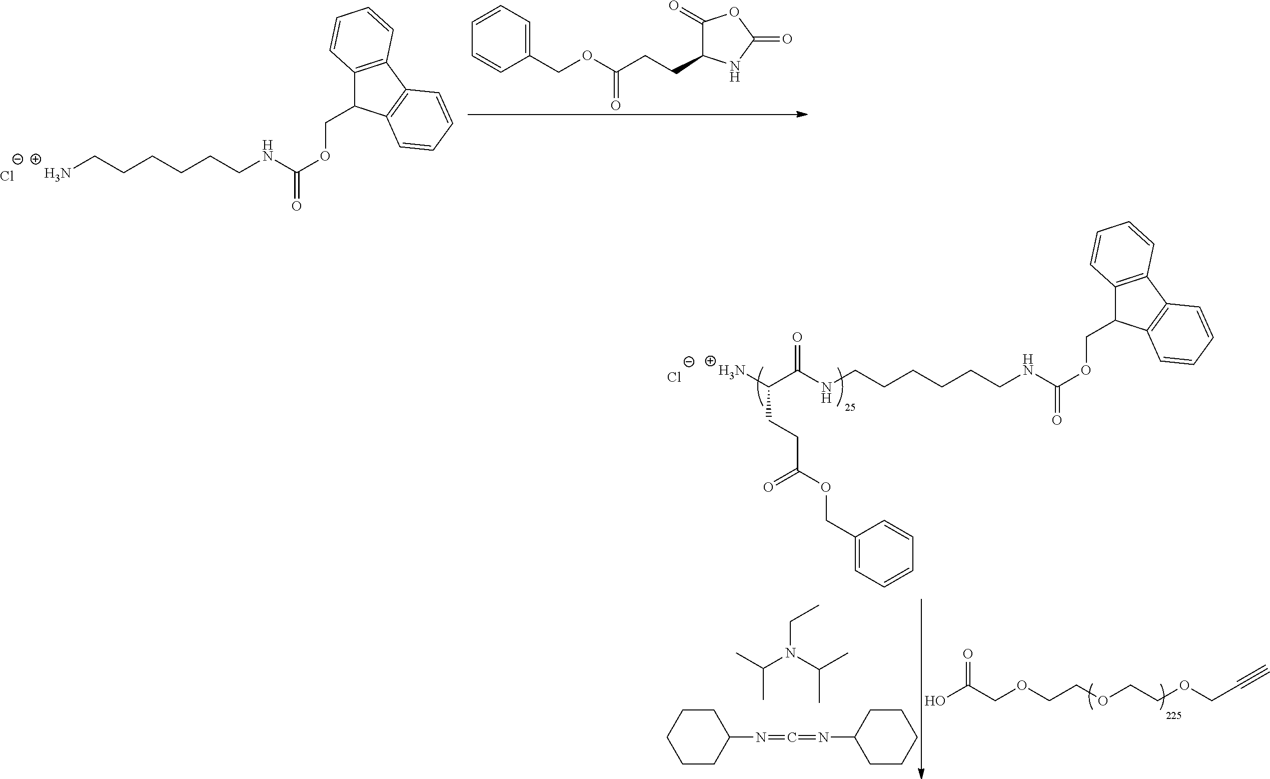

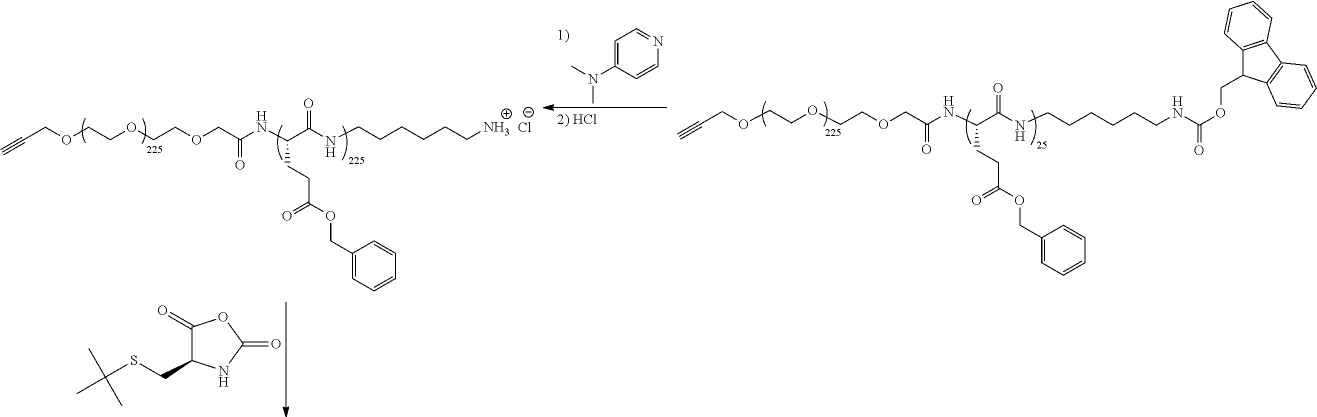

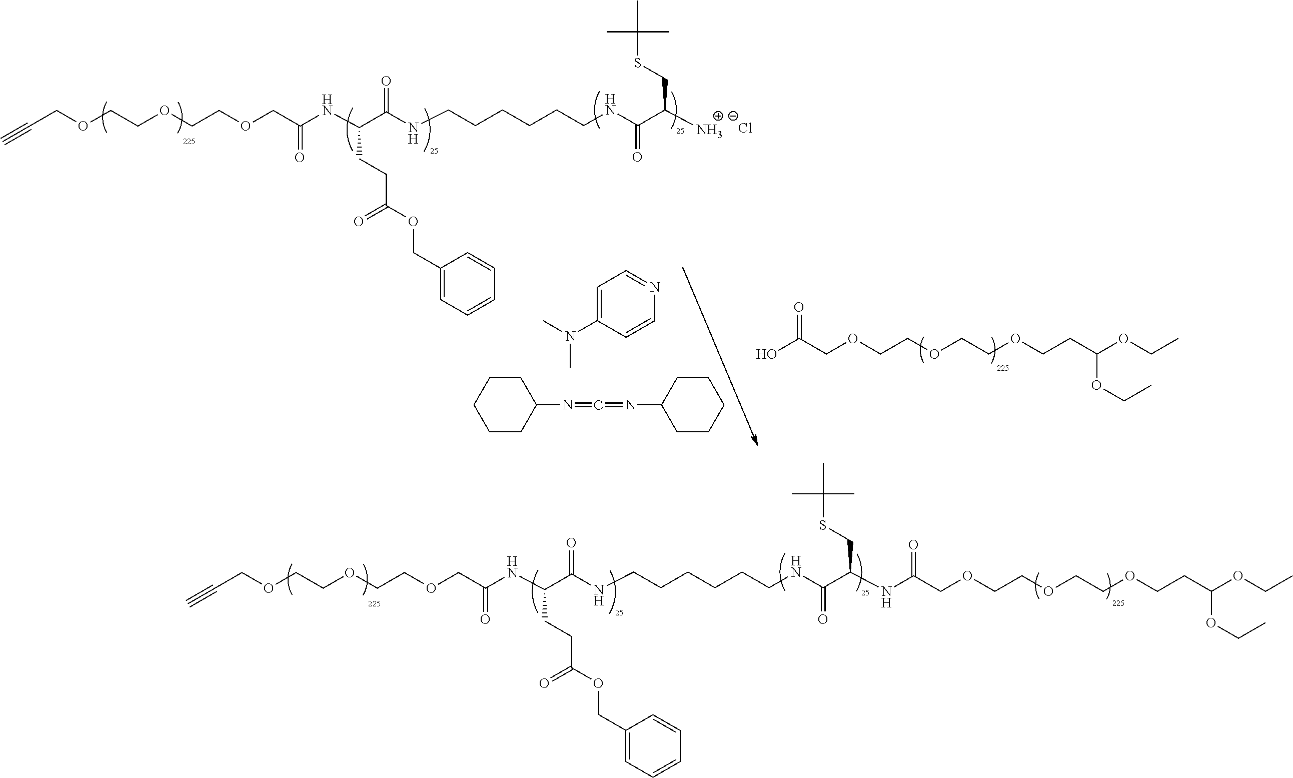

### Example 17

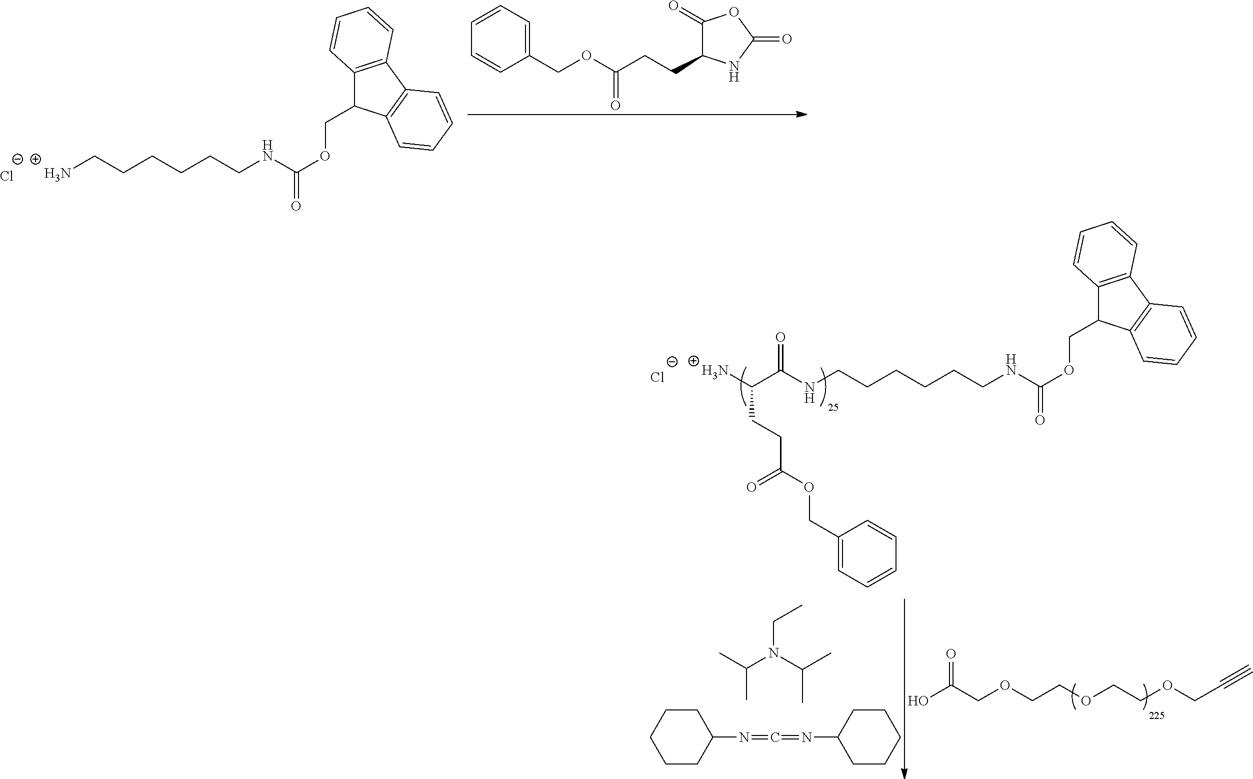

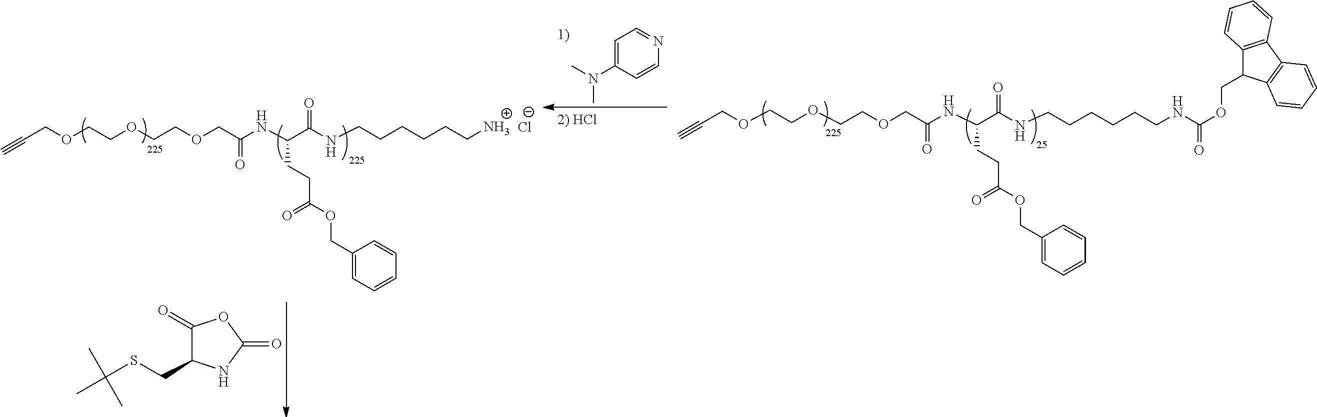

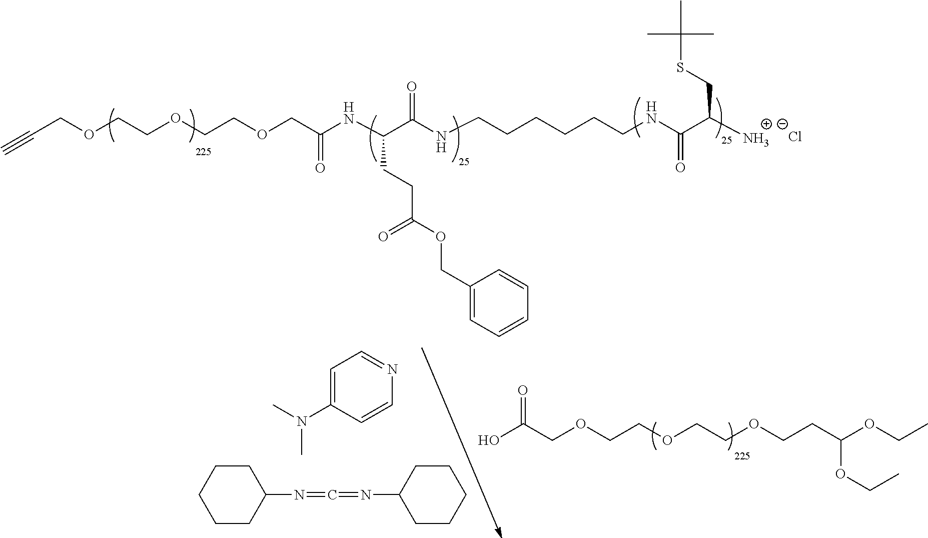

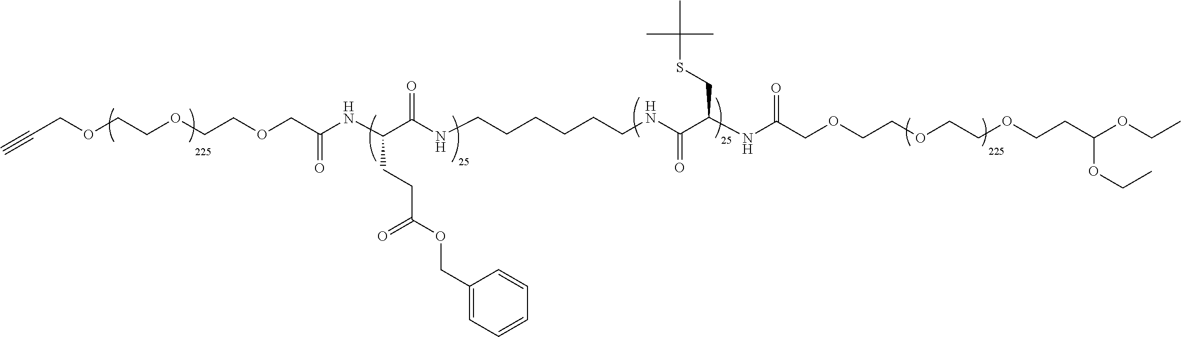

### Example 18

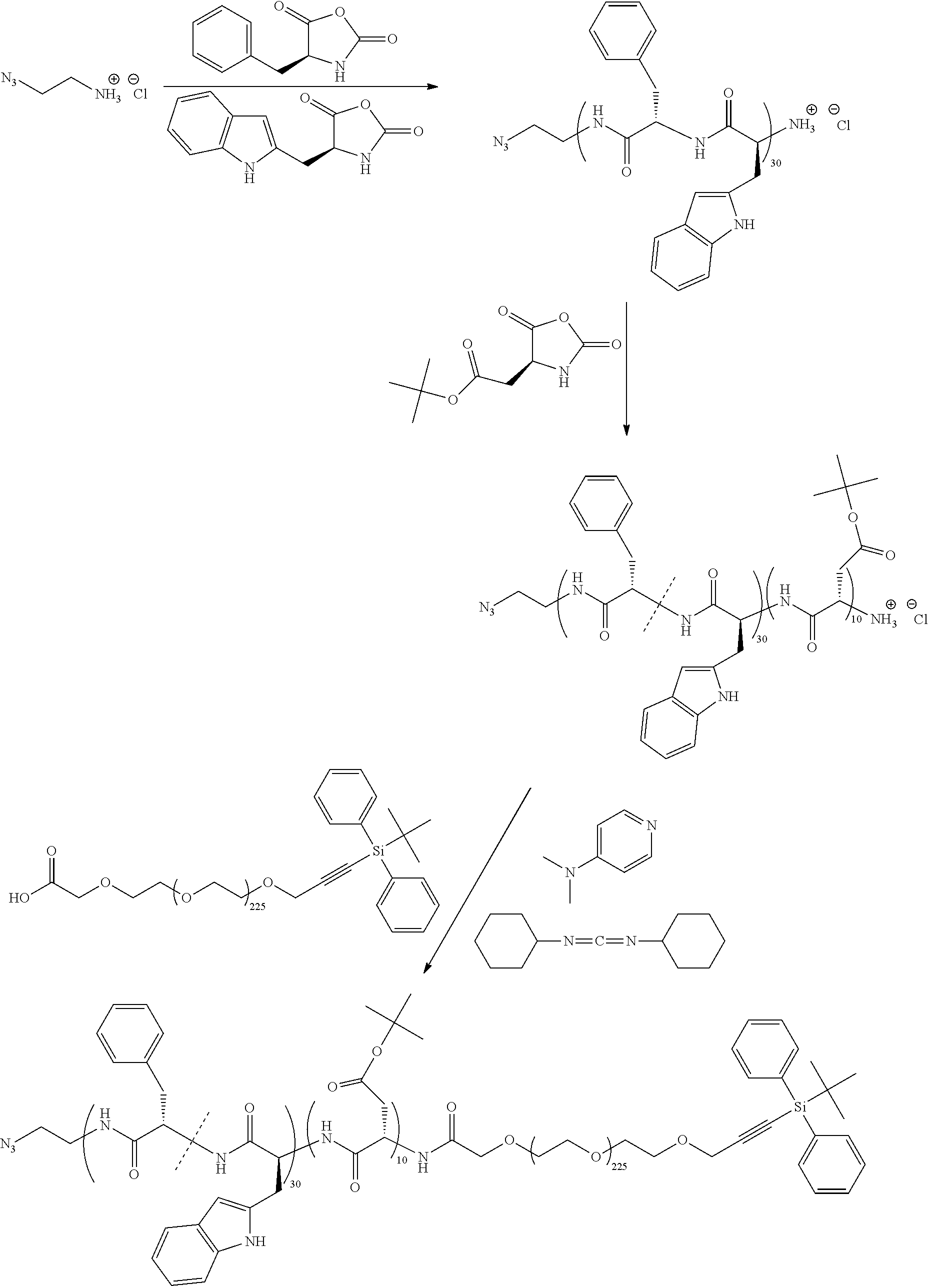

### Example 19

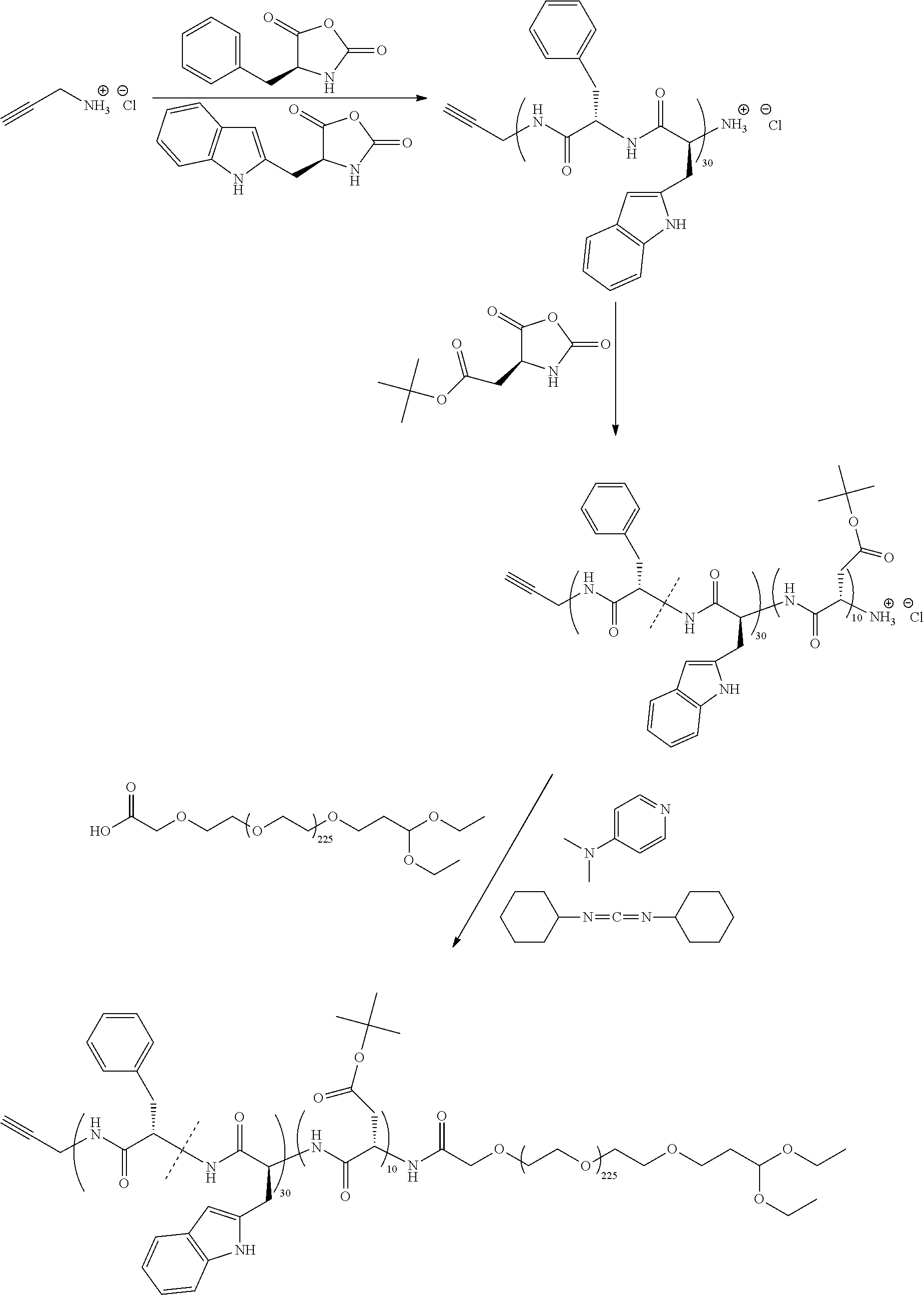

### Example 20

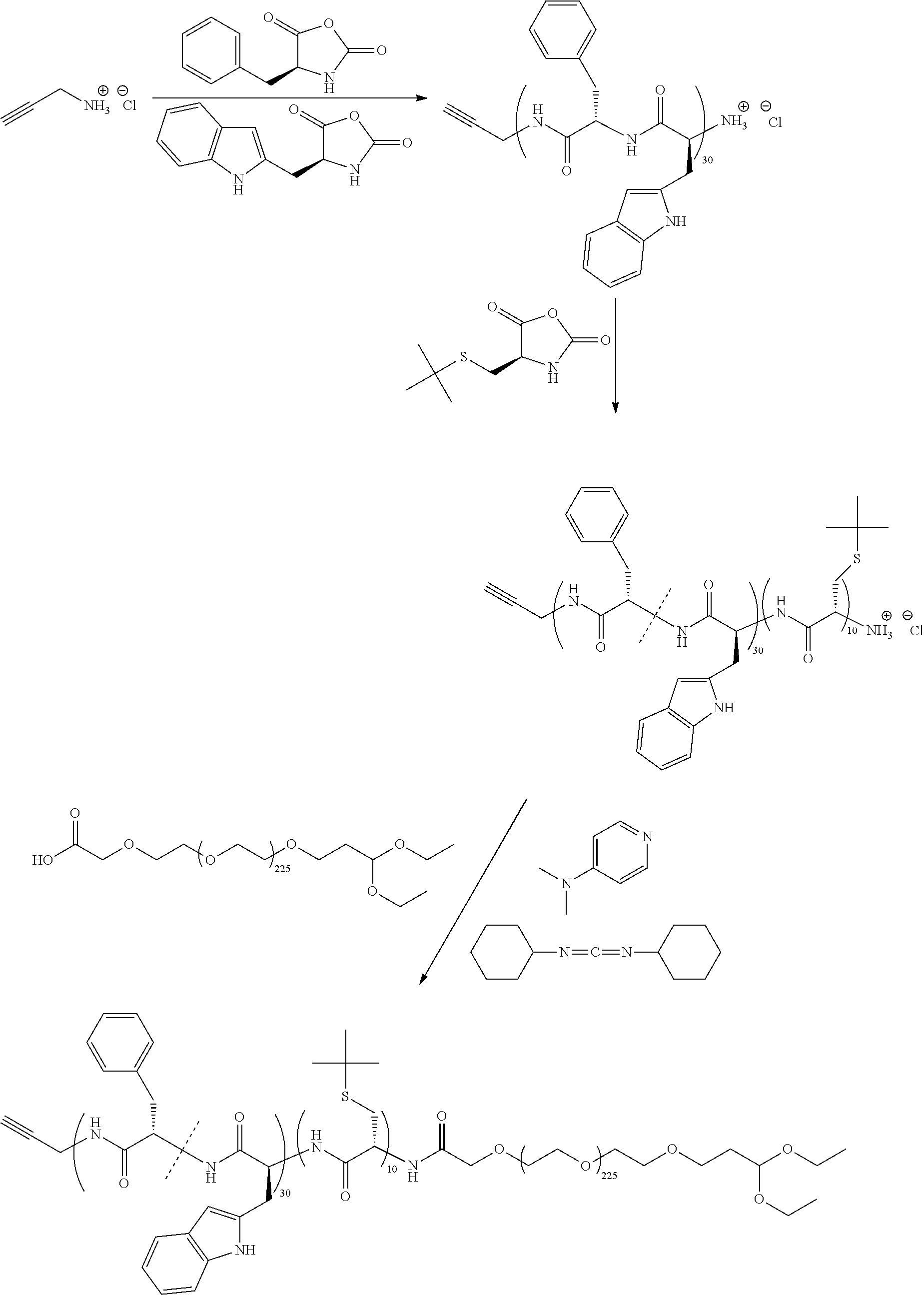

### Example 21

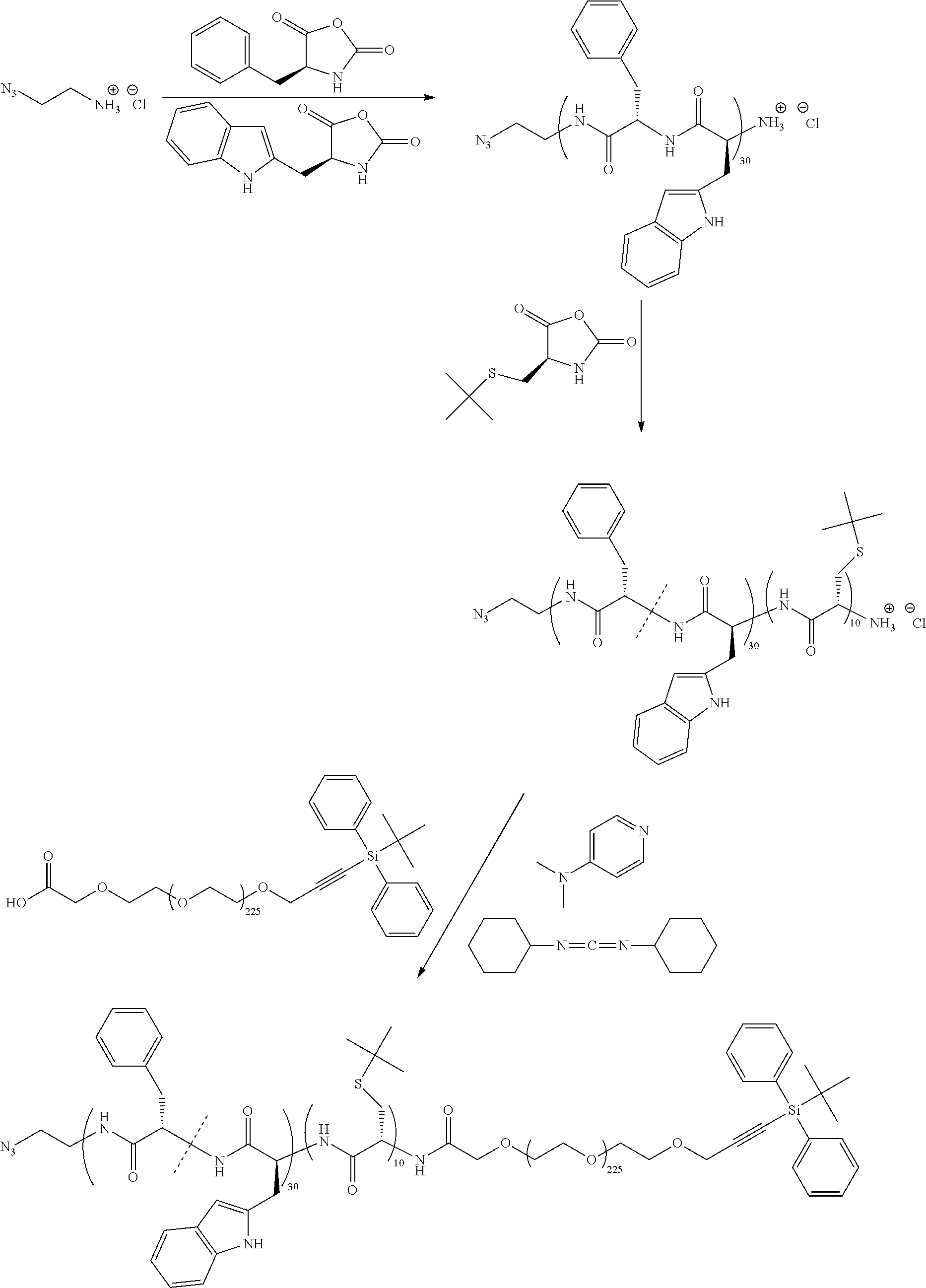

### Example 22

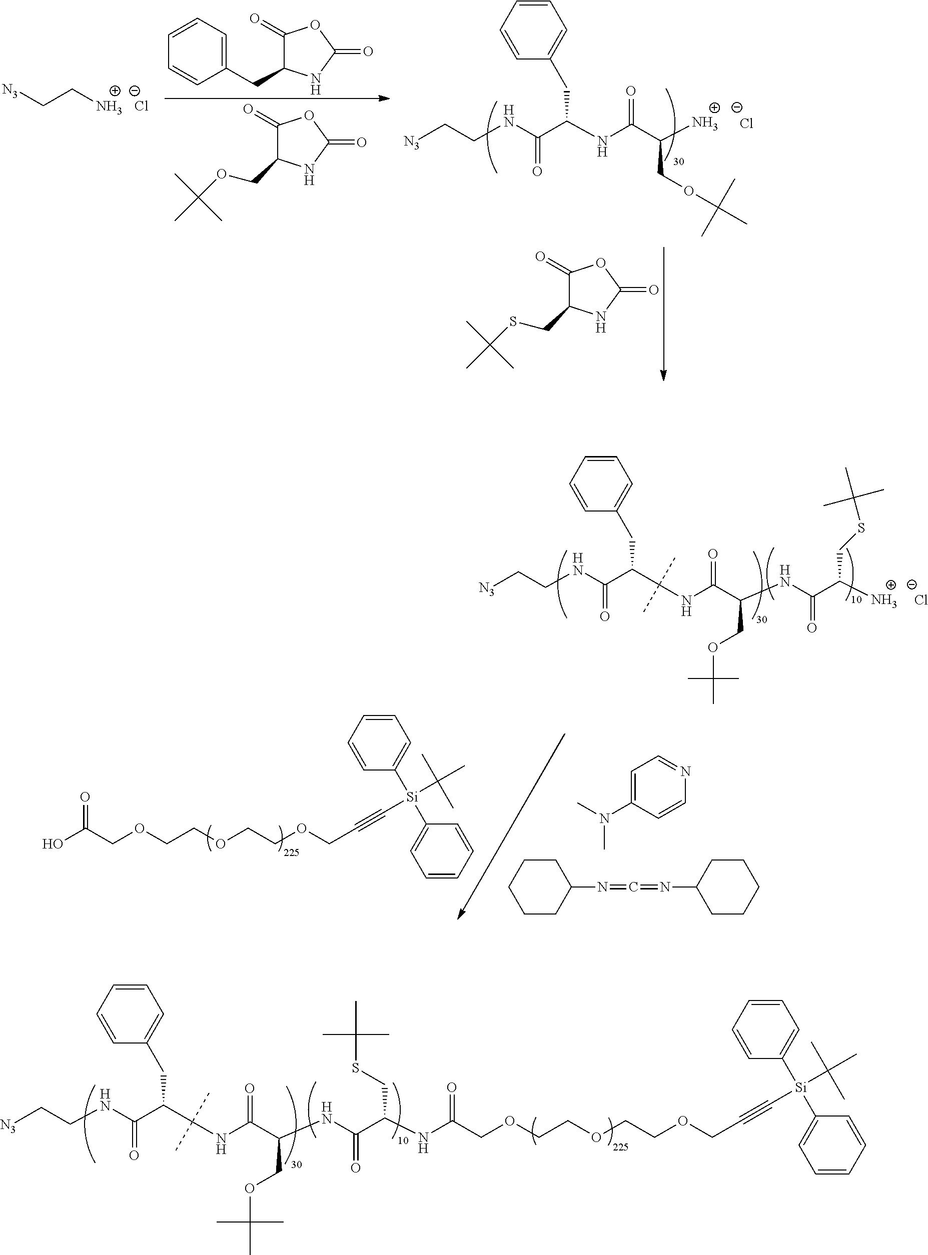

### Example 23

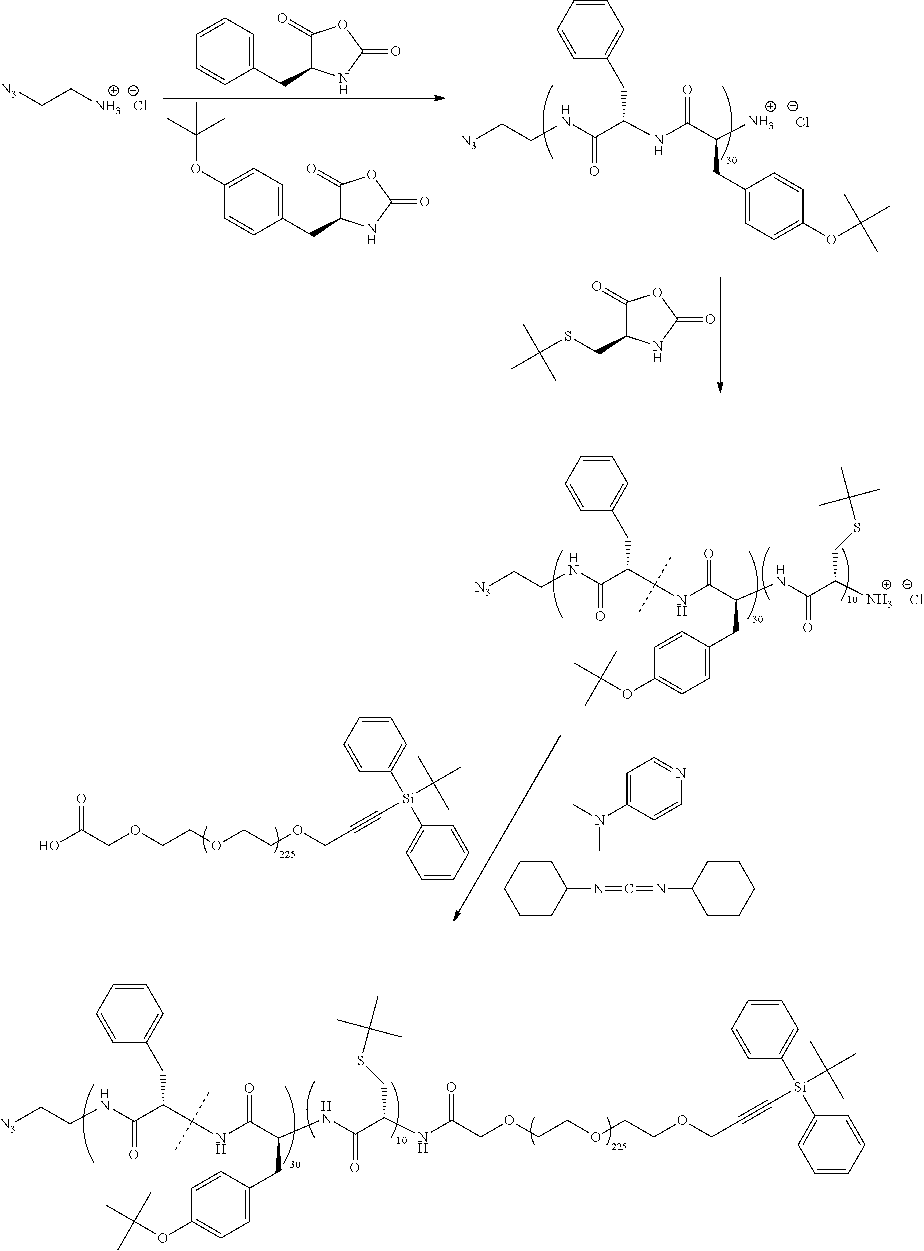

### Example 24

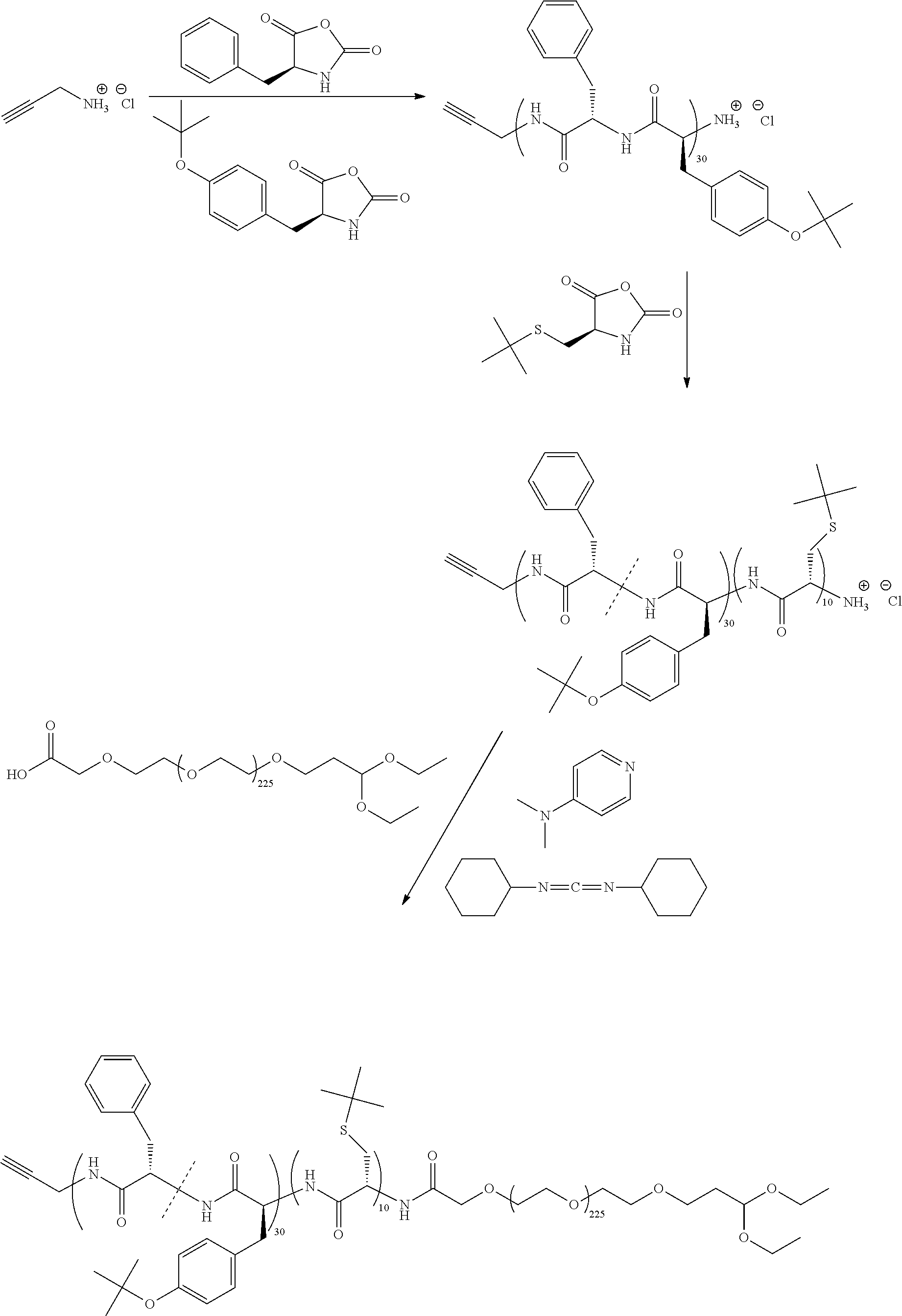

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method for preparing a multi-block copolymer comprising a non-polymeric core portion and one or more different poly(amino acid) blocks, wherein said method comprises the step of sequentially polymerizing one or more different cyclic amino acid monomers onto a non-polymeric amine salt wherein said polymerization is initiated by said amine salt.

2. The method according to claim 1, wherein said polymerization occurs by ring-opening polymerization of the cyclic amino acid monomers and wherein each cyclic amino acid monomer is an NCA.

3. The method according to claim 2, wherein said terminal amine salt is an acid addition salt formed with an inorganic acid selected from hydrochloric acid, hydrobromic acid, phosphoric acid, sulfuric acid or perchloric acid.

4. A method of preparing a block copolymer comprising one or more different poly(amino acid) blocks and a non-polymeric core moiety R¹, wherein said method comprises the steps of:
(a) providing a compound of formula I: 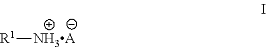
wherein:
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and
A is a suitable acid anion,
(b) polymerizing a first cyclic amino acid monomer onto the amine salt terminal end of formula I; and
(c) optionally polymerizing additional cyclic amino acid monomers onto the living polymer end.

5. The method according to claim 4, wherein R¹ is a C₁₋₁₀ aliphatic group optionally substituted with —N₃, —CN, an amino group, a mono-protected amino group, a di-protected amino group, a protected aldehyde group, a protected hydroxyl group, a protected carboxylic acid group, a protected thiol group, an optionally substituted aliphatic group, or a detectable moiety.

6. The method according to claim 4, wherein R¹ comprises a group suitable for Click chemistry.

7. The method according to claim 4, wherein R¹ is an optionally substituted group selected from a 5-7 membered saturated, partially unsaturated, or aryl monocyclic ring having 0-2 heteroatoms independently selected from oxygen, nitrogen, or sulfur; a 9-10 membered saturated, partially unsaturated, or aryl bicyclic ring having 0-3 heteroatoms independently selected from oxygen, nitrogen, or sulfur; or a 13-14 membered saturated, partially unsaturated, or aryl tricyclic ring having 0-3 heteroatoms independently selected from oxygen, nitrogen, or sulfur.

8. The method according to claim 7, wherein R¹ is a 5-6 membered saturated, partially unsaturated, or aryl monocyclic ring having 0-3 heteroatoms independently selected from oxygen, nitrogen, or sulfur, optionally substituted with —N₃, —CN, an amino group, a mono-protected amino group, a di-protected amino group, a protected aldehyde group, a protected hydroxyl group, a protected carboxylic acid group, a protected thiol group, an optionally substituted aliphatic group, or a detectable moiety.

9. A method for preparing a multi-block copolymer of formula II: 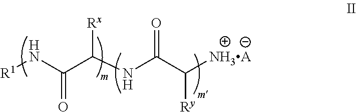
wherein:
m is 1-1000;
m′ is 0-1000;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
A is a suitable acid anion; and
R^{x} and R^{y} are each independently a natural or unnatural amino acid side-chain group, wherein R^{x} and R^{y} are different from each other,
wherein said method comprises the steps of:
(a) providing a compound of formula I: 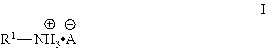
wherein:
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and
A is a suitable acid anion,
(b) polymerizing a first cyclic amino acid monomer onto the amine salt terminal end of formula I, wherein said first cyclic amino acid monomer comprises R^{x}; and
(c) optionally polymerizing a second cyclic amino acid monomer, comprising R^{y}, onto the living polymer end, wherein said second cyclic amino acid monomer is different from said first cyclic amino acid monomer.

10. The method according to claim 9, wherein one of R^{x} and R^{y} is a hydrophilic, or crosslinkable, amino acid side-chain group and the other of R^{x} and R^{y} is a hydrophobic, or ionic amino acid side-chain group.

11. The method according to claim 10, wherein said hydrophilic amino acid side-chain group is an aspartic acid side chain, a glutamic acid side-chain, or a suitably protected aspartic acid or glutamic acid side-chain, a lysine side-chain, an arginine side-chain, or a suitably protected lysine or arginine side-chain.

12. The method according to claim 10, wherein said hydrophobic amino acid side-chain group is a phenylalanine side-chain, a tyrosine side-chain or a suitably protected tyrosine side-chain, a serine side-chain or a suitably protected serine side-chain, a threonine side-chain or a suitably protected threonine side-chain, an alanine side-chain, a valine side-chain, a leucine side-chain, a tryptophan side-chain, a proline side-chain, a benzyl or alkyl glutamate side-chain, or a benzyl or alkyl aspartate side-chain, or mixtures thereof.

13. A compound of formula II: 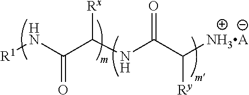
wherein:
m is 1-1000;
m′ is 0-1000;
R^{x} and R^{y} are each independently a natural or unnatural amino acid side-chain group, wherein R^{x} and R^{y} are different from each other;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and
A is a suitable acid anion.

14. The compound according to claim 13, wherein one of R^{x} and R^{y} is a hydrophilic, or crosslinkable, amino acid side-chain group and the other of R^{x} and R^{y} is a hydrophobic, or ionic, amino acid side-chain group.

15. The method according to claim 14, wherein said hydrophilic amino acid side-chain group is an aspartic acid side chain, a glutamic acid side-chain, or a suitably protected aspartic acid or glutamic acid side-chain, a lysine side-chain, an arginine side-chain, or a suitably protected lysine or arginine side-chain.

16. The method according to claim 14, wherein said hydrophobic amino acid side-chain group is a phenylalanine side-chain, a tyrosine side-chain or a suitably protected tyrosine side-chain, a serine side-chain or a suitably protected serine side-chain, a threonine side-chain or a suitably protected threonine side-chain, an alanine side-chain, a valine side-chain, a leucine side-chain, a tryptophan side-chain, a proline side-chain, a benzyl or alkyl glutamate side-chain, or a benzyl or alkyl aspartate side-chain, or mixtures thereof.

17. The compound according to claim 13, wherein R¹ is a C₁₋₁₀ aliphatic group optionally substituted with —N₃, —CN, an amino group, a mono-protected amino group, a di-protected amino group, a protected aldehyde group, a protected hydroxyl group, a protected carboxylic acid group, a protected thiol group, an optionally substituted aliphatic group, or a detectable moiety.

18. The compound according to claim 13, wherein R¹ comprises a group suitable for Click chemistry.

19. The compound according to claim 13, wherein R¹ is an optionally substituted group selected from a 5-7 membered saturated, partially unsaturated, or aryl monocyclic ring having 0-2 heteroatoms independently selected from oxygen, nitrogen, or sulfur; a 9-10 membered saturated, partially unsaturated, or aryl bicyclic ring having 0-3 heteroatoms independently selected from oxygen, nitrogen, or sulfur; or a 13-14 membered saturated, partially unsaturated, or aryl tricyclic ring having 0-3 heteroatoms independently selected from oxygen, nitrogen, or sulfur.

20. The method according to claim 19, wherein R¹ is a 5-6 membered saturated, partially unsaturated, or aryl monocyclic ring having 0-3 heteroatoms independently selected from oxygen, nitrogen, or sulfur, optionally substituted with —N₃, —CN, an amino group, a mono-protected amino group, a di-protected amino group, a protected aldehyde group, a protected hydroxyl group, a protected carboxylic acid group, a protected thiol group, an optionally substituted aliphatic group, or a detectable moiety.

21. A compound of formula II′: 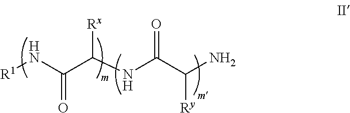
wherein:
m is 1-1000;
m′ is 0-1000;
R^{x} and R^{y} are each independently a natural or unnatural amino acid side-chain group, wherein R^{x} and R^{y} are different from each other; and
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group.

22.-30. (canceled)

31. The compound according to claim 22, wherein said compound is of formula III-a:

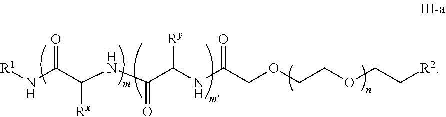

32. A method for preparing the compound according to claim 31, wherein said method comprises the steps of:
(a) providing a compound of formula I: 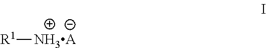
wherein:
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group; and
A is a suitable acid anion,
(b) polymerizing a first cyclic amino acid monomer onto the amine salt terminal end of formula I, wherein said first cyclic amino acid monomer comprises R^{x};
(c) optionally polymerizing a second cyclic amino acid monomer, comprising R^{y}, onto the living polymer end, wherein said second cyclic amino acid monomer is different from said first cyclic amino acid monomer; and
(d) coupling onto the amine terminus a compound of formula

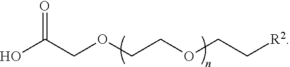

33. The compound according to claim 32, wherein one of R^{x} and R^{y} is a hydrophilic, or crosslinkable, amino acid side-chain group and the other of R^{x} and R^{y} is a hydrophobic, or ionic amino, acid side-chain group.

34. The method according to claim 33, wherein said hydrophilic amino acid side-chain group is an aspartic acid side chain, a glutamic acid side-chain, or a suitably protected aspartic acid or glutamic acid side-chain, a lysine side-chain, an arginine side-chain, or a suitably protected lysine or arginine side-chain.

35. The method according to claim 33 wherein said hydrophobic amino acid side-chain group is a phenylalanine side-chain, a tyrosine side-chain or a suitably protected tyrosine side-chain, a serine side-chain or a suitably protected serine side-chain, a threonine side-chain or a suitably protected threonine side-chain, an alanine side-chain, a valine side-chain, a leucine side-chain, a tryptophan side-chain, a proline side-chain, a benzyl or alkyl glutamate side-chain, or a benzyl or alkyl aspartate side-chain, or mixtures thereof.

36. The compound according to claim 32, wherein R¹ is a C₁₋₁₀ aliphatic group optionally substituted with —N₃, —CN, an amino group, a mono-protected amino group, a di-protected amino group, a protected aldehyde group, a protected hydroxyl group, a protected carboxylic acid group, a protected thiol group, an optionally substituted aliphatic group, or a detectable moiety.

37. The compound according to claim 32, wherein R¹ comprises a group suitable for Click chemistry.

38. The compound according to claim 32, wherein R¹ is an optionally substituted group selected from a 5-7 membered saturated, partially unsaturated, or aryl monocyclic ring having 0-2 heteroatoms independently selected from oxygen, nitrogen, or sulfur; a 9-10 membered saturated, partially unsaturated, or aryl bicyclic ring having 0-3 heteroatoms independently selected from oxygen, nitrogen, or sulfur; or a 13-14 membered saturated, partially unsaturated, or aryl tricyclic ring having 0-3 heteroatoms independently selected from oxygen, nitrogen, or sulfur.

39. The method according to claim 38, wherein R¹ is a 5-6 membered saturated, partially unsaturated, or aryl monocyclic ring having 0-3 heteroatoms independently selected from oxygen, nitrogen, or sulfur, optionally substituted with —N₃, —CN, an amino group, a mono-protected amino group, a di-protected amino group, a protected aldehyde group, a protected hydroxyl group, a protected carboxylic acid group, a protected thiol group, an optionally substituted aliphatic group, or a detectable moiety.

40. The method according to claim 32, wherein R² is selected from:

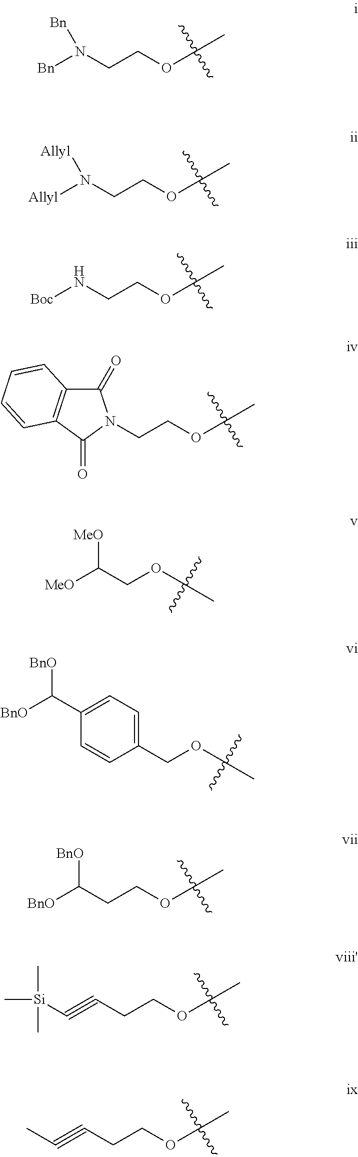

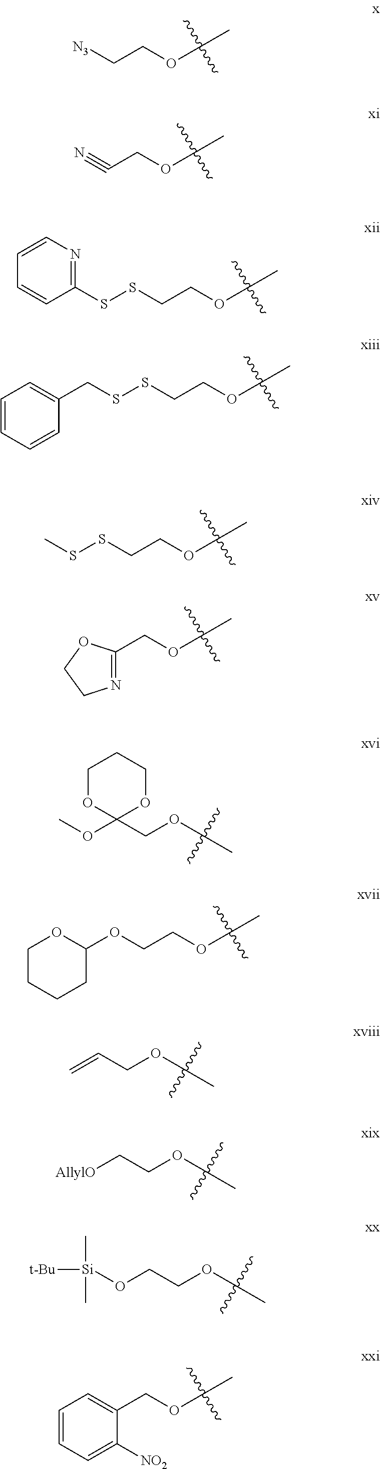

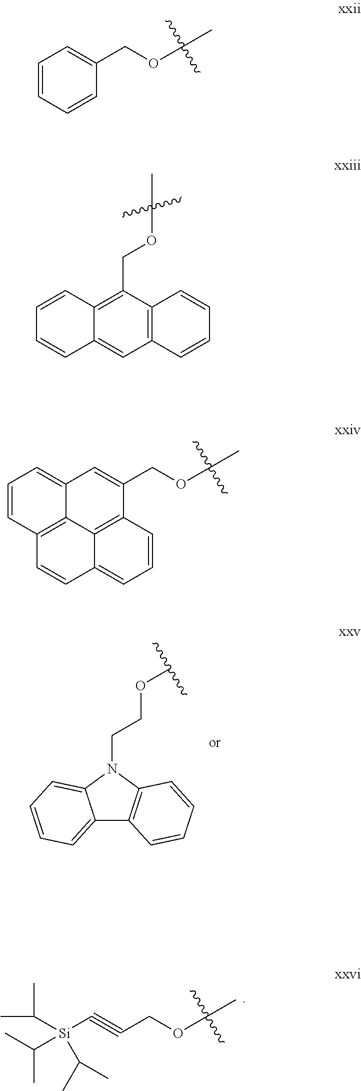

41. A compound of formula IV: 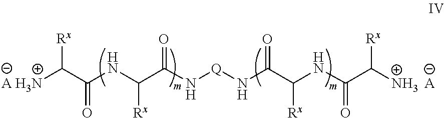
wherein:
each m is independently 1-1000;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
A is a suitable acid anion; and
R^{x} is a natural or unnatural amino acid side-chain group.

42. A compound of formula IVb: 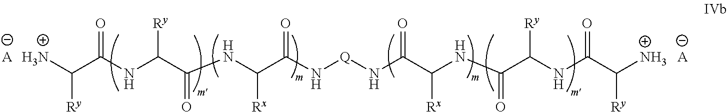
wherein:
m is 1-1000;
m′ is 0-1000;
R¹ is an optionally substituted group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
A is a suitable acid anion; and
R^{x} and R^{y} are each independently a natural or unnatural amino acid side-chain group, wherein R^{x} and R^{y} are different from each other.

43. A compound of formula V: 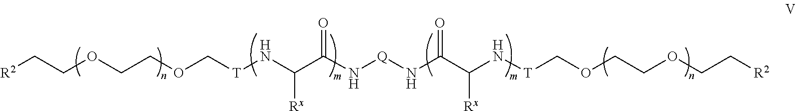
wherein:
each n is independently 10-2500;
each m is independently 1-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} is a natural or unnatural amino acid side-chain group;
each T is independently a valence bond or a bivalent, saturated or unsaturated, straight or branched C₁₋₁₂ alkylene chain, wherein 0-6 methylene units of Q are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each R is independently hydrogen or an optionally substituted aliphatic group; and
each R² is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³;
q and r are each independently 0-4;
each R³ is independently an optionally substituted group selected from aliphatic, a 5-8-membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
two R³ on the same nitrogen atom are taken together with said nitrogen atom to form an optionally substituted 4-7-membered saturated, partially unsaturated, or aryl ring having 1-4 heteroatoms independently selected from nitrogen, oxygen, or sulfur; and
R⁴ is hydrogen, halogen, N₃, CN, a mono-protected amine, a di-protected amine, a protected aldehyde, a protected hydroxyl, a protected carboxylic acid, a protected thiol, or an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety.

44. The compound according to claim 43, wherein said compound is of formula V-a:

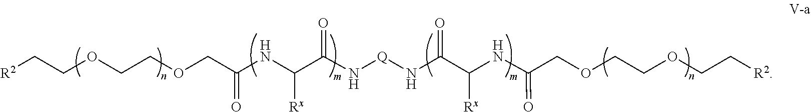

45. A compound of formula VI: 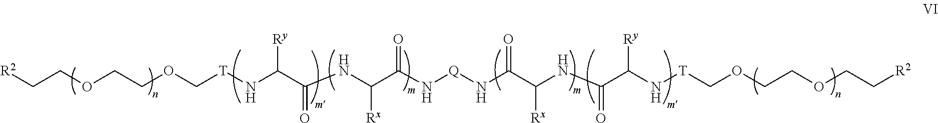
wherein:
each n is independently 10-2500;
m is 1-1000;
m′ is 0-1000;
Q is an optionally substituted bivalent group selected from a C₁₋₆ aliphatic group, a 3-7 membered saturated, partially unsaturated, or aryl monocyclic ring having 0-3 heteroatoms independently selected from oxygen, nitrogen, or sulfur, an 8-10 membered saturated, partially unsaturated, or aryl bicyclic ring having 0-5 heteroatoms independently selected from oxygen, nitrogen, or sulfur, a 12-14 membered saturated, partially unsaturated, or aryl tricyclic ring having 0-5 heteroatoms independently selected from oxygen, nitrogen, or sulfur, or an amine-terminal dendritic group;
R^{x} and R^{y} are each independently a natural or unnatural amino acid side-chain group, wherein R^{x} and R^{y} are different from each other;
each T is independently a valence bond or a bivalent, saturated or unsaturated, straight or branched C₁₋₁₂ alkylene chain, wherein 0-6 methylene units of Q are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
each R² is halogen, N₃, CN, a mono-protected amine, a di-protected amine, a protected hydroxyl, a protected aldehyde, a protected thiol, —NHR³, —N(R³)₂, —SR³, —O(CH₂CH₂O)_{q}(CH₂)ᵣR⁴, —OC(O)R³, or —OS(O)₂R³;
q and r are each independently 0-4;
each R³ is independently an optionally substituted group selected from aliphatic, a 5-8-membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety, or:
two R³ on the same nitrogen atom are taken together with said nitrogen atom to form an optionally substituted 4-7-membered saturated, partially unsaturated, or aryl ring having 1-4 heteroatoms independently selected from nitrogen, oxygen, or sulfur; and
R⁴ is hydrogen, halogen, N₃, CN, a mono-protected amine, a di-protected amine, a protected aldehyde, a protected hydroxyl, a protected carboxylic acid, a protected thiol, or an optionally substituted group selected from aliphatic, a 5-8-membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety.

46. The compound according to claim 45, wherein said compound is of formula VI-a: [chemical structure]


SEQUENCE LISTING (2 sequences)

SEQ ID NO: 1 - protein, 5 aa, Artificial Sequence
  Chemically synthesized polypeptide
WXXXX

SEQ ID NO: 2 - protein, 5 aa, Artificial Sequence
  Chemically synthesized polypeptide
XXWXX